(19) United States
(12) Patent Application Publication
(10) Pub. No.: US 2023/0025756 A1
(43) Pub. Date: Jan. 26, 2023

(54) INDUCTION OF REGULATORY B CELLS FOR THE TREATMENT OF IMMUNE-MEDIATED DISEASES

(71) Applicant: THE REGENTS OF THE UNIVERSITY OF COLORADO, A BODY CORPORATE, Denver, CO (US)

(72) Inventor: Susan Boackle Childress, Denver, CO (US)

(21) Appl. No.: 17/675,731

(22) Filed: Feb. 18, 2022

Related U.S. Application Data
(63) Continuation of application No. 15/459,873, filed on Mar. 15, 2017, now abandoned.
(60) Provisional application No. 62/308,434, filed on Mar. 15, 2016.

(51) Int. Cl. A61K 35/17; C07K 16/28; C12N 5/0781; C12N 15/113

(52) U.S. Cl. CPC A61K 35/17; C07K 16/2803; C12N 5/0635; C07K 16/2896; C12N 15/113; G01N 33/5052

(57) ABSTRACT

The present invention contemplates that complement receptor 1 and 2 may play a role in the induction of regulatory B cells under inflammatory conditions that accompany immune-mediated diseases. For example, long noncoding RNA may regulation transcription of the complement receptor I gene, thereby resulting in an induction and/or suppression of regulatory B cells. Such long noncoding RNA in mature B cells may be specifically targeted to modify complement receptor 1 levels and induce or suppress the generation of antigen-specific regulatory B cells, thereby modifying the course of immune mediated diseases including, but not limited to, autoimmune disease, cancer, and infection.

### PRIORITY

This Continuation application is a Continuation of application Ser. No. 15/459,873, filed Mar. 15, 2017, which claims the benefit of U.S. Provisional Application Ser. No. 62/308,434, filed Mar. 15, 2016. These applications are incorporated herein by reference in their entirety for all purposes.

### STATEMENT OF GOVERNMENT SUPPORT

This invention was made with government support under R01 A1070983, K24 A1078004 and T32 AR007534 all awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on May 11, 2017, is named 18562lrg.txt and is 13,708 bytes in size.

### FIELD

This invention is related to the field of diagnosis and treatment of autoimmune diseases. For example, systemic lupus erythematosus development may be controlled by a protective CR2 SNP that modifies B cell responses to a relevant disease-associated autoantigen and confers decreased risk of disease. Overexpression of CR1 augments the generation of regulatory B cells in response to complement-opsonized antigens and contributes to the induction of antigen specific immune tolerance.

### BACKGROUND

Autoimmune diseases affect more than 2% of the US population, with effects on morbidity and mortality and associated health care costs that rival those of cancer and heart disease. The biggest challenge in caring for these patients is preventing the permanent organ damage that results from misdirected attack on self by the immune system. Historically, treatment has involved global immunosuppression, but this approach leads to increased risk of infection and malignancy, requires ongoing treatment, and often has limited efficacy. As our understanding of the immunological mechanisms that drive these diseases has grown, the impetus has been to develop specific therapies that restore immune tolerance to disease-associated autoantigens.

Autoimmune diseases develop when individuals who are at increased genetic risk have environmental exposures that alter their immune function, resulting in an imbalance between regulation and activation of immune responses and the loss of self-tolerance. The earliest sign of autoimmunity is the production of autoantibodies, and in many cases these can precede the first manifestation of disease by years. During this preclinical period, therapies that restore tolerance to self-antigen may prevent evolution to clinically apparent disease and the tissue and organ damage associated with this. However, intervention at a phase when symptoms are absent and time to disease onset may be long requires a relatively benign treatment with acceptably low toxicity. These “benign” therapies may be discovered through the study of alleles identified in genetic studies to decrease risk of disease, as these variants highlight pathways that are ideal therapeutic targets. The existence of these disease-protective alleles in individuals who are otherwise healthy suggests that targeting the pathways that they modify will be safe and effective even at the earliest stages of disease development.

Systemic lupus erythematosus (SLE) is a systemic autoimmune disease resulting in inflammation in multiple organs that can cause permanent damage. Approximately 1 in 1000 Americans have lupus, 90% of whom are women between the ages of 15 and 45. Non-Caucasian minorities have both a higher prevalence and increased severity of disease. A number of immunologic abnormalities have been linked to the pathogenesis of lupus, including defects in clearance of apoptotic cells and immune complexes, altered function of B and T cells, and dysregulation of the innate immune system with chronic production of cytokines such as the type I interferons. Both genetic and environmental factors are involved in the development of lupus, and the exact sequence of events that leads to its onset is not well understood. Furthermore, it is characterized by intermittent remissions and exacerbations, with the latter being difficult to predict and often resulting in progressive organ damage over time.

Only a single new drug has been developed for lupus in the last 50 years and the current drugs that are available are toxic and not always effective; as a result, the burden of morbidity and mortality associated with this disease is striking, with a 10-year survival rate of ˜70%. Tsokos G., “Systemic lupus erythematosus” N Engl J Med 365: 2110-2121. What is needed is the identification of new therapeutics for lupus as a public health priority.

### SUMMARY

This invention is related to the field of diagnosis and treatment of autoimmune diseases. For example, systemic lupus erythematosus development may be controlled by a protective CR2 SNP that modifies B cell responses to a relevant disease-associated autoantigen and confers decreased risk of disease. Overexpression of CR1 augments the generation of regulatory B cells in response to complement-opsonized antigens and contributes to the induction of antigen specific immune tolerance.

In one embodiment, the present invention contemplates an expression construct comprising a human DNA sequence encoding a human long noncoding RNA (lncRNA) sequence. In one embodiment, the long noncoding RNA molecule comprises a sequence encoded by intron 1 of a complement receptor 2 (CR2) gene. In one embodiment, the encoded intron 1 sequence comprises a guanine→adenine transposition at position rs1876453. In one embodiment, the long noncoding RNA comprises SEQ 9124. In one embodiment, the expression construct comprises an inducible expression element. In one embodiment, the inducible expression element is reversible.

In one embodiment, the present invention contemplates a method, comprising, a) providing, i) an expression construct comprising a sequence encoding a long noncoding RNA (lncRNA), wherein said long non-coding RNA is encoded by intron 1 of a complement receptor 2 (CR2) gene, and ii) a stem cell, and b) transfecting said expression construct into said stem cell. In one embodiment, the encoded intron 1 sequence comprises a guanine→adenine transposition at position rs1876453. In one embodiment, the long noncoding RNA is lncRNA 9124. In one embodiment, the method further comprises the step of differentiating the stem cell into a B cell. In one embodiment, the B cell is induced into a regulatory B cell. In one embodiment, the method further comprises administering said stem cell to a patient at risk for developing an autoimmune disease. In one embodiment, the method further comprises administering said B cell to a patient at risk for developing an autoimmune disease. In one embodiment, the method further comprises administering said regulatory B cell to a patient at risk for developing an autoimmune disease. In one embodiment, the expression construct further comprises a reversible expression induction element. In one embodiment, the reversible expression induction element modulates said long noncoding RNA expression. In one embodiment, said administering further comprises an inflammatory stimulus. In one embodiment, said inflammatory stimulus includes, but is not limited to, LPS, CpG, interferon alpha/beta and a vaccine. In one embodiment, the autoimmune disease is systemic lupus erythematosus. In one embodiment, the autoimmune disease is type 1 diabetes. In one embodiment, said transfecting increases CR1 levels in said B cell.

In one embodiment, the present invention contemplates a composition comprising a nucleic acid sequence encoding a long noncoding RNA (lncRNA) comprising a stabilizer molecule, wherein said long non-coding RNA is encoded by intron 1 of a complement receptor 2 (CR2) gene and a pharmaceutical formulation. In one embodiment, the stabilizer molecule comprises a poly-A tail. In one embodiment, the stabilizer molecule is a protein. In one embodiment, the protein is a cationic protein. In one embodiment, the stabilizer molecule comprises a mutated lncRNA codon enriched in cytosine or uracil. In one embodiment, the stabilizer molecule comprises a nucleotide analogue. In one embodiment, the stabilizer molecule comprises a 5′-blocking group. In one embodiment, the stabilizer molecule comprises a 3′ blocking group. In one embodiment, the pharmaceutical formulation comprises a buffer salt solution. In one embodiment, the pharmaceutical formulation may include, but is not limited to, microparticles, nanoparticles or liposomes. In one embodiment, the lncRNA comprises a guanine→adenine transposition at position rs1876453. In one embodiment, the long noncoding RNA is lncRNA 9124. In one embodiment, the lncRNA is an unspliced lncRNA. In one embodiment, the lncRNA is a spliced lncRNA.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” as used herein, in the context of any of any assay measurements refers to +/−5% of a given measurement.

The term “long non-coding ribonucleic acid”, “long non-coding RNA” or “lncRNA” refers to a ribonucleic acid sequence that is encoded within a genomic intronic or intergenic region. Such lncRNAs are not transcribed into proteins but act directly to regulate various activities including, but not limited to, transcription or translation. For example, an lncRNA may be exemplified by an aptamer that regulates transcription rates of a particular gene or allele.

The term “CTCF” as used herein refers to a transcriptional repressor also known as an 11-zinc finger protein or CCCTC-binding factor encoded by the CTCF gene. Filippova et al., “An exceptionally conserved transcriptional repressor, CTCF, employs different combinations of zinc fingers to bind diverged promoter sequences of avian and mammalian c-myc oncogenes”. Mol. Cell. Biol. 16(6): 2802-2813 (1996); and Rubio et al., “CTCF physically links cohesin to chromatin” Proc. Natl. Acad. Sci. U.S.A. 105(24): 8309-8314 (2008). The term “suspected of having”, as used herein, refers a medical condition or set of medical conditions (e.g., preliminary symptoms) exhibited by a patient that is insufficient to provide a differential diagnosis. Nonetheless, the exhibited condition(s) would justify further testing (e.g., autoantibody testing) to obtain further information on which to base a diagnosis.

As used herein, the term “inducing differentiation” in reference to a cell refers to changing the default cell type (phenotype) to a non-default cell type (phenotype). Thus “inducing differentiation in a stem cell” refers to inducing the cell to divide into progeny cells with characteristics that are different from the stem cell, such as genotype (i.e. change in gene expression as determined by genetic analysis such as a microarray) and/or phenotype (i.e. change in expression of a protein, such as PAX6 or a set of proteins, such as HMB45 positive (+) while negative (−) for SOX10).

As used herein, the term “contacting” cells with a compound of the present invention refers to, for example, by placing the compound in a location that will allow it to touch the cell or by infusing a compound into a patient, in order to produce “contacted” cells. The contacting may be accomplished using any suitable method. For example, in one embodiment, contacting is by adding the compound to a tube of cells. Contacting may also be accomplished by adding the compound to a culture of the cells.

As used herein, the term “stem cell” refers to a cell that is totipotent or pluripotent or multipotent and are capable of differentiating into one or more different cell types, such as stem cells isolated from organs, for example, skin stem cells, bone marrow stem cells, or stem cells isolated from placenta or umbilical cord.

As used herein, the term “totipotent” refers to an ability of a cell to differentiate into any type of cell in a differentiated organism, as well as a cell of extra embryonic materials, such as placenta, etc.

As used herein, the tern “pluripotent” refers to a cell line capable of differentiating into any differentiated cell type.

As used herein, the term “multipotent” refers to a cell line capable of differentiating into at least two differentiated cell types.

As used herein, the term “differentiation” as used with respect to cells in a differentiating cell system refers to the process by which cells differentiate from one cell type (e.g., a multipotent, totipotent or pluripotent differentiable cell) to another cell type such as a target differentiated cell.

As used herein, the term “cell differentiation” in reference to a pathway refers to a process by which a less specialized cell (i.e. stem cell) develops or matures or differentiates to possess a more distinct form and/or function into a more specialized cell or differentiated cell, (i.e. white blood cell, a B cell, a regulatory B cell, a T cell etc.).

The term “at risk for” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “inhibitory compound” as used herein, refers to any compound capable of interacting with (i.e., for example, attaching, binding etc. to) a binding partner under conditions such that the binding partner becomes unresponsive to its natural ligands. Inhibitory compounds may include, but are not limited to, small organic molecules, antibodies, and proteins/peptides.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand.

The term “portion” when used in reference to a nucleotide sequence refers to fragments of that nucleotide sequence. The fragments may range in size from 5 nucleotide residues to the entire nucleotide sequence minus one nucleic acid residue.

As used herein, the term “antisense” is used in reference to RNA sequences which are complementary to a specific RNA sequence (e.g., mRNA). Antisense RNA may be produced by any method, including synthesis by splicing the gene(s) of interest in a reverse orientation to a viral promoter which permits the synthesis of a coding strand. Once introduced into a cell, this transcribed strand combines with natural mRNA produced by the cell to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

As used herein, the terms “siRNA” refers to either small interfering RNA, short interfering RNA, or silencing RNA. Generally, siRNA comprises a class of double-stranded RNA molecules, approximately 20-25 nucleotides in length. Most notably, siRNA is involved in RNA interference (RNAi) pathways and/or RNAi-related pathways. wherein the compounds interfere with gene expression.

As used herein, the term “shRNA” refers to any small hairpin RNA or short hairpin RNA. Although it is not necessary to understand the mechanism of an invention, it is believed that any sequence of RNA that makes a tight hairpin turn can be used to silence gene expression via RNA interference. Typically, shRNA uses a vector stably introduced into a cell genome and is constitutively expressed by a compatible promoter. The shRNA hairpin structure may also cleaved into siRNA, which may then become bound to the RNA-induced silencing complex (RISC). This complex binds to and cleaves mRNAs which match the siRNA that is bound to it.

As used herein, the term “microRNA”, “miRNA”, or “pRNA” refers to any single-stranded RNA molecules of approximately 21-23 nucleotides in length, which regulate gene expression. miRNAs may be encoded by genes from whose DNA they are transcribed but miRNAs are not translated into protein (i.e. they are non-coding RNAs). Each primary transcript (a pri-miRNA) is processed into a short stem-loop structure called a pre-miRNA and finally into a functional miRNA. Mature miRNA molecules are partially complementary to one or more messenger RNA (mRNA) molecules, and their main function is to down-regulate gene expression.

A “variant” of a nucleotide is defined as a novel nucleotide sequence which differs from a reference oligonucleotide by having deletions, insertions and substitutions. These may be detected using a variety of methods (e.g., sequencing, hybridization assays etc.).

A “deletion” is defined as a change in either nucleotide or amino acid sequence in which one or more nucleotides or amino acid residues, respectively, are absent.

An “insertion” or “addition” is that change in a nucleotide or amino acid sequence which has resulted in the addition of one or more nucleotides or amino acid residues, respectively, as compared to, for example, the naturally occurring Bacillus anthracis BclA.

A “substitution” results from the replacement of one or more nucleotides or amino acids by different nucleotides or amino acids, respectively.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc.

The term “in operable combination” as used herein, refers to any linkage of nucleic acid sequences in such a manner that a nucleic acid molecule capable of directing the transcription of a given gene and/or the synthesis of a desired protein molecule is produced. Regulatory sequences may be operably combined to an open reading frame including but not limited to initiation signals such as start (i.e., ATG) and stop codons, promoters which may be constitutive (i.e., continuously active) or inducible, as well as enhancers to increase the efficiency of expression, and transcription termination signals.

Transcriptional control signals in eukaryotes comprise “promoter” and “enhancer” elements. Promoters and enhancers consist of short arrays of DNA sequences that interact specifically with cellular proteins involved in transcription. Maniatis, T. et al., Science 236:1237 (1987). Promoter and enhancer elements have been isolated from a variety of eukaryotic sources including genes in plant, yeast, insect and mammalian cells and viruses (analogous control elements, i.e., promoters, are also found in prokaryotes). The selection of a particular promoter and enhancer depends on what cell type is to be used to express the protein of interest.

The term “transfection” or “transfected” refers to the introduction of foreign DNA into a cell.

As used herein, the terms “nucleic acid molecule encoding”, “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

As used herein, the term “structural gene” refers to a DNA sequence coding for RNA or a protein. In contrast, “regulatory genes” are structural genes which encode products which control the expression of other genes (e.g., transcription factors).

As used herein, the term “gene” means the deoxyribonucleotide sequences comprising the coding region of a structural gene and including sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogeneous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term, “expression construct” refers to any nucleic acid sequence (i.e., for example, deoxyribonucleic acid (DNA) or ribonucleic acid (RNA) sequence) that is configured to be expressed with either cis or trans regulatory expression factors.

### BRIEF DESCRIPTION OF THE FIGURES

The file of this patent contains at least one drawing executed in color. Copies of this patent with color drawings will be provided by the Patent and Trademark Office upon request and payment of the necessary fee.

FIGS. 1A-1B illustrates embodiments of various regulatory elements in the CR2-CR1 genomic locus. FIG. 1A: illustrates Coding RNA transcripts. The rs1876453 SNP is located 97 nucleotides from the 5′ end of the CR2 gene intron 1. FIG. 1B: illustrates Long noncoding RNA transcripts.

FIGS. 1C-1E illustrate various embodiments disclosed herein. FIG. 1C: illustrates histone modifications, as determined by ChIP-seq. The H3K4Me3 histone mark is associated with poised or active promoters, the H3K4me1 histone mark is associated with enhancers and with DNA regions downstream of transcription sites, and the H3K27Ac histone mark may enhance transcription by blocking the spread of the repressive histone mark H3K27Me3. Cell lines used to generate the histone data are GM12878 (EBVtransformed B cells), HSMM (skeletal muscle), HUVEC (umbilical epithelial cells), K562 (erythroleukemia cells), and NHEK (epidermal keratinocytes). FIG. 1D: DNAse I hypersensitivity sites derived by DNase-seq. FIG. 1E: Partial list of transcription factor binding sites, as determined by ChIP-seq. Data shown are from the GM12878 EBV-transformed B cell line and primary CD20+B cells. (UCSC Genome Browser (Human February 2009 [GRCh37/hg19] Assembly; genome.ucsc.edu), with a custom track (C) added to show lncRNA from NONCODE v.4).

FIG. 2 represents exemplary data showing allele-specific differences in expression of lncRNA 9124.

FIGS. 3A-3B represents exemplary data showing that lncRNA 9124 is increased in subjects with the minor protective allele (3A) at rs1876453 (N=3 minor, N=3 major). FIG. 3A: normalized to U6 snRNA; FIG. 3B: normalized to β-actin).

FIG. 4 represents exemplary data showing SLE disease activity tracked by released biomarkers.

FIGS. 5A-5B represents exemplary data showing the relationships between autoantibody development and SLE diagnosis (5A) and SLE symptom manifestation (5B).

FIG. 6 represents one embodiment of complement-mediated organ damage as a result of SLE. (www.studyblue.com)

FIG. 7 represents exemplary data showing that the rs1876453 SNP increases CR1 gene expression compared to controls.

FIG. 8 represents exemplary data showing that the rs1876453 SNP induced CR1 gene expression is specific for B cells.

FIG. 9 represents exemplary data showing the location of the rs1876453 SNP in relation to CTCF transcriptional regulatory binding site.

FIG. 10 represents exemplary data showing preferential binding of CTCF to the CTCF transcriptional regulatory binding site in the presence of the G allele of the rs1876453 SNP.

FIG. 11 illustrates various embodiments of long noncoding RNA sequences in the CR2-CR1 genomic locus.

FIG. 12 represents exemplary data showing the tissue distribution of lncRNA 9124.

FIGS. 13A-13B represent an illustration of one embodiment of a B cell complement receptor system. For example, on a B cell membrane, CR2 may be found in two complexes: one containing CR1 (2) and a second containing CD19 and CD81 (3). Tuveson et al., 1991 “Molecular interactions of complement receptors on B lymphocytes: a CR1/CR2 complex distinct from the CR2/CD19 complex” J. Exp. Med. 173:1083-1089; and Matsumoto et al., 1991 “Intersection of the complement and immune systems: a signal transduction complex of the B lymphocyte-containing complement receptor type 2 and CD19. J. Exp. Med. 173:55-64, respectively. FIG. 13A: is a schematic representation illustrating where CR1 binds C3b and acts as a cofactor for the factor I (fI)-mediated cleavage of C3b to iC3b and C3dg. CR1 is an essential cofactor for the final cleavage of iC3b to C3dg, the specific ligand for CR2. FIG. 13B: is a schematic representation illustrating when CR2 is coligated with the B cell receptor (BCR), it lowers the threshold for B cell activation. In contrast, CR1 communicates an inhibitory signal to the B cell when coligated with the BCR. Although others have examined the effects of coligation of either CR1 or CR2 with the BCR, the effects of engaging both receptors, as would occur during the normal processing of C3b opsonized ligands, are not known.

FIGS. 14A-14E presents exemplary data showing an association of SNPs in the CR2 region with reduced risk of anti-dsDNA autoantibodies. FIG. 14A: illustrates genomic structure of CR2 regions (14A) and positions of genetic variants. FIG. 14B: Allelic P value (−log 10P value) of each genetic variant with dsDNA autoantibodies plotted against its position as a circle (genotyped) or triangle (imputed) for European American (EA) as illustrated in FIG. 14B, African American (AA) as illustrated in FIG. 14C and Hispanic (HS) as illustrated in FIG. 14D. Genetic variants are colored according to their linkage disequilibrium (LD) strength (r2) with rs1876453. Arrow marks rs1876453. FIG. 14E: Transancestral meta-analysis P values using a fixed (red) and random (blue) model.

FIG. 15 represents exemplary data showing that a protective SNP lies within a putative enhancer and is 78 nucleotides 5′ of a long noncoding RNA transcript. The protective CR2 SNP, rs1876453 (indicated by arrow), lies within an intronic region with histone marks characteristic of active enhancers (H3K27ac, H3K4m2) that were identified in primary B cells (CD20+) and EBV-transformed B cell lines (GM12878). The chromatin is open surrounding the SNP (DNase hypersensitivity track), and specifically over the protective SNP in primary B cells. Nine additional B cell lines appeared similar to GM12878 with respect to DNase hypersensitivity in this region. Binding of CTCF, which organizes the chromatin into looping domains, is also shown.

FIGS. 16A-16C presents exemplary data showing that the rs1876453 SNP is associated with increased C3b binding and processing. FIG. 16A: represents data of human peripheral blood mononuclear cells were isolated from whole blood and incubated with various concentrations of C3b tetramers or an anti-CR1 antibody. B cells were identified using a fluorochrome-labeled anti-CD19. The minor allele at rs1876453 is associated with elevated B cell CR1 levels (27.5%) and increased C3b binding (26% at 1 ug; 30.6% at 0.1 ug). FIG. 16B: illustrates data demonstrating equal cell equivalents of B cell lysates from individuals homozygous for the major or minor allele at rs1876453 were incubated with C3b tetramers+factor I. To confirm a role for CR1 in the processing of C3b, an antibody to CR1 that partially blocks this processing function (4D6) was preincubated with lysates in some samples. After 30 minutes, biotinylated fragments of the C3a′ chain were separated by SDS-PAGE, transferred to a nitrocellulose filter, and detected using HRP-SA. FIG. 16C: illustrates data demonstrating equal cell equivalents of B cell lysates were also run on a separate gel under reducing conditions, transferred and blotted with anti-CR1 to determine the amount of CR1 present. More C3dg was generated in the presence of the minor allele, suggesting that augmented processing occurs with more CR1.

FIGS. 17A-17F present exemplary data showing binding of C3b-tetramers is increased on B cells expressing higher levels of CR1 and remains higher after Factor I cleavage of C3b. C3b- and C3dg-tetramers were prepared by mixing biot-C3b or biot-C3dg with PE-labeled streptavidin. C3b tetramers were incubated with purified B cells for 0, 30, 45, and 60 minutes at 37° C. in the presence (17A, 17C) or absence (17B, 17D) of factor I to test CR1 cofactor activity. Binding of C3dg tetramers to B cells was included as a control (17E). Cells were stained with a Ghost Dye for dead cell exclusion, AF647-labeled anti-CD19 to mark B cells, and FITC anti-C3 to detect uncleaved C3b. Cells were fixed and analyzed by flow cytometry. Tetramer binding was determined by PE fluorescence (17A, 17B) and presence of uncleaved C3b was determined by FITC fluorescence (17C, 17D). CR1 and CR2 levels were determined using an AF488-labeled antibody to CR1 or CR2 (17F). Median FI for PE FMO (tetramer control) was 14.8 and for FITC anti-C3 to C3d tetramers (anti-C3b control) was 195. rs1876453 major GG=circles, minor heterozygote GA=squares.

FIGS. 18A-18D represent exemplary data showing the effects of coligation of CR1 and BCR during C3b inactivation. Tetramers were prepared by incubating biot-C3b, biot-C3d, and biotinylated anti-BCR with PE-labeled streptavidin. Tetramers were incubated with purified human B cells for 0, 30, 45, and 60 minutes at 37° C. in the presence or absence of factor I. Cells were stained with a Ghost Dye for dead cell exclusion, AF647-labeled anti-CD19 to mark B cells, and a FITC-labeled antibody to C3 that recognizes C3b but not iC3b or C3d to enable analysis of C3b processing. Cells were fixed and analyzed by flow cytometry. As seen in FIG. 7, inactivation of C3b by factor I resulted in release of tetramers from cells (A), but this was reversed when anti-BCR was incorporated in the tetramers (18B). Binding of tetramers to the B cells was highest when the BCR and CR1 were coligated (18B), despite higher relative amounts of C3b being present in tetramers that bound CR1 alone (18C vs 18D). After incubation of the cells with Factor I, ability of the tetramers to be detected by the antibody to C3 was reduced, confirming C3b cleavage (18C, 18D). Anti-C3 bound at higher levels to cells exposed to C3b-tetramers+factor I versus C3d-tetramers. Since similar amounts of PE-labeled streptavidin were used in preparing the tetramers, this likely means that not all of the C3b was converted to iC3b or that the final conversion step to C3d has not yet occurred with the residual binding representing background binding to iC3b.

FIGS. 19A-19D represent exemplary data showing an induction of regulatory B cells by CR1/CR2 ligation contributes to suppression of autoimmune disease by Complete Freund's Adjuvant (CFA) in mice prone to autoimmune diabetes. Female NOD mice sufficient (19A) or deficient (19B) for CR1 and CR2 (n=16 per group) were given a subcutaneous injection of saline emulsified CFA at 4 weeks of age and monitored for diabetes development until 30 weeks of age. Diabetes was defined as a reading of >200 mg/dl of blood glucose for three consecutive days. In a separate experiment, female NOD mice sufficient or deficient for CR1 and CR2 were given a subcutaneous injection of CFA at 6 weeks of age and spleens harvested 9 days later for measurement of B10 cells (n=7 per group). Splenocytes were incubated with lipopolysaccharide, PMA, ionomycin, and monensin for 5 hours. After blocking Fcγ receptors and incubating with a cell viability dye, cells were stained with antibodies to CD19, then permeabilized and stained with antibodies to IL10. B10 cells as a percentage of live B cells (C) and absolute numbers of splenic B10 cells (19D) are shown, with each point representing a single mouse and the lines and error bars representing mean+SD. P values were determined using a Kaplan-Meier estimate (19A, 19B) or ordinary one-way ANOVA test (19C, 19D) and a p value of <0.05 is considered significant. Female mice were studied because disease onset is earlier and more penetrant.

FIGS. 20A-20F represent exemplary data showing that a deficiency of CR1 and CR2 in the SNF1 model of murine lupus accelerates renal failure and death. (NZB×SWR)F1 lupus-prone mice sufficient or deficient for CR1/CR2 were monitored for 74 weeks. The survival curves were significantly different, with median survival of CR1/CR2-sufficient mice of 54 weeks and of CR1/CR2-deficient mice of 36 weeks. Although there was no significant difference in proteinuria between the two groups, there was a trend towards earlier mortality after proteinuria onset in the CR1/CR2-deficient mice. Nonetheless, mortality was due to renal failure as significantly more CR1/CR2-deficient mice still alive at 32 weeks had increased BUN levels, with the median significantly higher than CR1/CR2-sufficient mice (20D), but this was not associated with significantly increased levels of anti-dsDNA (20E) or antichromatin antibodies (20F) although there was a trend towards decreased anti-chromatin in mice deficient in CR1/CR2. In FIGS. 20D-20F, each point represents a single mouse, and the line and error bars represent median+interquartile range. FIGS. 20A-20C were analyzed using the Gehan-Breslow-Wilcoxon test, whereas FIGS. 20D-20F were analyzed using the Mann-Whitney test. A p value of <0.05 is considered significant.

FIG. 21 presents exemplary data showing putative B cell enhancer domains 5′ of CR2 and in the first intron are actively transcribed. Published Global Run-On sequencing (GRO-Seq) data from a human colon cancer cell line (HCT116) exposed to DMSO or Nutlin-3 are shown in the first two tracks, followed by unpublished GRO-Seq data from EBV-transformed B cells from a Down syndrome quartet (child with Down syndrome, unaffected brother, mother, and father). The next four tracks show unpublished RNA-Seq data from the EBV-transformed B cells from the Down syndrome quartet. Sense transcription is shown in blue and anti-sense transcription is shown in red. Bidirectional transcriptional activity is shown in the first intron of CR2 as well as in the intergenic region 5′ of CR2, consistent with the presence of active enhancers in these regions that appear to be B cell-specific. GRO-Seq is able to identify enhancer RNA, whereas RNA-Seq typically cannot detect these unstable low frequency transcripts.

FIG. 22 represents exemplary data showing additional putative B cell enhancers that lie upstream of CR2. DNA accessibility and histone marks suggestive of active enhancers are present in the intergenic region upstream of CR2. IRF4 and STAT3 binding sites are also present in this region. The CTCF binding site in this region makes contact with the CTCF binding site in proximal CR1.

FIG. 23 represents exemplary data showing that putative B cell enhancer domains 5′ of CR2 contact the 5′ end of CR1 to form a chromatin loop. Data shown are from a Hi-C map of the GM12878 human lymphoblastoid cell line at a resolution of ˜1 kB. Yellow lines show contact domains, and blue squares indicate pairs of loci that show significantly closer proximity with one another than with the loci lying between them and correspond to the anchor points for chromatin loops. Gene location is shown in the outside track, then CTCF orientation (green=sense; red=antisense), then DNase hypersensitivity sites, then histone marks associated with active enhancers. The vast majority of loops are anchored at a pair of convergent CTCF binding sites. DAF=Decay accelerating factor (CD55), C4BPA and C4BPB=C4 binding protein A or B.

### DETAILED DESCRIPTION

Embodiments of these inventions relate to the field of diagnosis and treatment of autoimmune diseases. For example, systemic lupus erythematosus development may be controlled by a protective CR2 SNP that modifies B cell responses to a relevant disease-associated autoantigen and confers decreased risk of disease. Overexpression of CR1 augments the generation of regulatory B cells in response to complement-opsonized antigens and contributes to the induction of antigen specific immune tolerance.

In one embodiment, the present invention contemplates nucleic acid constructs and methods of treatment relating to modifying the course or onset of Systemic Lupus Erythematosus (SLE). In one embodiment, the course modification comprises delaying time of onset. In one embodiment, the course modification comprises delaying time of flare. In one embodiment, the course modification comprises interrupting flare. In one embodiment, the time of onset is delayed by preventing the production of double stranded deoxyribonucleic acid (dsDNA) autoantibodies. In one embodiment, the flare is interrupted by terminating the production of dsDNA autoantibodies.

Although it is not necessary to understand the mechanism of an invention, it is believed that a patient cell comprises a protective CR2 allele (e.g., a rs1876453 SNP sequence) whose expression results in the production of complement receptor 1 (CR1/CD35). The CR1/CD35 receptor system is believed to alter the growth, development and/or function of regulatory B cells. It is believed that the SLE-susceptibility CR2 allele expression would begin at an immature/transitional developmental stage and then end when a particular maturation and/or functional stage is reached.

Additionally, variations of these CR1 and/or CR2 genetic constructs and methods may be used for treating other antibody mediated autoimmune disorders besides SLE by altering CR1 gene expression at specific stages of B cell maturation. Although it is not necessary to understand the mechanism of an invention, it is believed that CR1 gene transcription regulation may alter B cell function, especially regulatory B cell function, which in some cases may provide a tolerizing function and in other cases can result in self-deletion, silencing, or editing to remove antibody production for that B cell's antibodies. In one embodiment, the present invention contemplates a method comprising administering a lncRNA sequence to a B cell to induce self-deletion, silencing, editing, alteration of antigen presentation, or altered production of cytokines that modulate acute and chronic inflammatory conditions as well as cancer.

In one embodiment, the present invention contemplates nucleic acid constructs and methods for increasing CR1 gene expression in B cells. In one embodiment, CR1 gene expression is increased at a specific developmental point. In one embodiment, the increased CR1 gene expression alters regulatory B cell function. Although it is not necessary to understand the mechanism of an invention, it is believed that regulatory B cells negatively regulate the immune system, i.e. immune responses. For example, in mice, the absence/loss of regulatory B cells may be associated with an appearance of autoimmune disease symptoms. It is further believed that regulatory B cells can produce IL-10, which may reduce autoimmune disease initiation, onset or severity. IL-10, depending upon the situations, can either augment or suppress immune activation. For example, adoptive transfer of antigen primed regulatory IL-10⁺ B cells (i.e., B10 cells) reduces inflammation for collagen induced arthritis and reduced severity during EAE disease onset. Further, stimulation of regulatory B10 cells in vitro for transfusion into mouse autoimmune models may protect NOD mice from developing diabetes, or collagen-induced arthritis, etc.

In one embodiment, the present invention contemplates that the CR1 receptor may be upregulated in individuals possessing a protective CR2 gene intron 1 SNP rs1876453. This SNP may reduce the risk of lupus and other autoimmune diseases. In addition, a transcriptional program is demonstrated that leads to an induction of regulatory B cells. Finally, the efficacy of therapeutic targeting of the CR1 receptor on B cells is assessed as a means of inducing autoantigen-specific tolerance in individuals with lupus and other autoimmune diseases.

Complement Receptor 1 (CR1)

A recombinant cell expressing a CR1 has been reported, but was not shown to reside in, or be expressed from any B cell, or a regulatory B cell. The report ligated promoter DNA to a CR1-coding sequence to provide for increased expression of the CR1 protein. Recombinant CR1 receptors was then detected on the surface of transfected host cells. Expressed full length CR1 proteins are described for therapeutic use, including providing an effective dose of CR1 protein, and expression by hematopoietic stem cell progeny in gene therapy. While reductions in cell surface expression of CR1 were discussed as a potential therapeutic strategy, increased CR1 receptor expression as a possible treatment in a systemic lupus erythematosus (SLE) patient was not suggested. Fearon, et al., “Human C3b/C4b receptor (CR1)” WO 1991/005047 A1. A soluble human CR1 (sCR1) polypeptide (i.e. sCR1 and CR1-IgG) was administered for treating systemic lupus erythematosus. Additionally, a B cell mediated inflammatory response was inhibited by administering a retroviral expression vector (i.e. adenovirus, herpesvirus), encoding a soluble CR1 polypeptide or by administering cells containing the sCR1 expression vector. It was suggested that recombinant cells containing sequences of sCR1 may modulate B-cell mediated effects. Host cells used for expressing sCR1 include lymphocytes, e.g. splenocytes, e.g. collagen-reactive spleen cells and mouse DBA tst cells. As an example, DBA mouse splenocytes and fibroblast cells were transformed with a CR1 expression vector for expressing sCR1, or other CR1 sequences. Injections of these cells prevented collagen-induced arthritis development and ameliorated established disease development induced by collagen injection. Chernajovsky et al., “Immune modulation by polypeptides related to cr1.” WO 1998/045430 (1998).

Although CR1 has been shown to mediate inhibitory signals in human B cells (12), the interpretation of the results of previous functional studies is problematic. Józsi et al., 2002. “Complement receptor type 1 (CD35) mediates inhibitory signals in human B lymphocytes” J Immunol 2002:2782-2788. Both CR1 and CR2 are removed when CR2 is inactivated in mice, so the specific contribution of each receptor cannot be easily dissected using standard knockouts. In addition, the monoclonal antibodies and heat aggregated C3 used as CR1 ligands for in vitro functional studies in humans cannot be processed to the CR2 ligands iC3b and C3d and thus do not reflect what occurs in vivo when a B cell encounters a C3b-opsonized immune complex.

In order to determine the effects of the ˜30% increase in B cell CR1 levels seen in individuals with the protective SNP, a reagent was developed including a biotinylated C3b (biot-C3b) bound to streptavidin to form biot-C3b tetramers that can be processed in a physiological manner. The biot-C3b may be prepared by incubating purified human C3 with factor B and factor D (Complement Technology, Inc., Tyler, Tex.) in the presence of Mg2+ and EZ-link maleimide-PEG2-biotin (Pierce Biotechnology, Rockford, Ill.) to allow site-specific biotinylation of the free sulfhydryl group in C3b that is exposed after spontaneous thioester hydrolysis. As a result, the C3b “tags” the streptavidin in the same way that C3b opsonizes immune complexes, and it can then be bound by CR1 and processed to C3d.

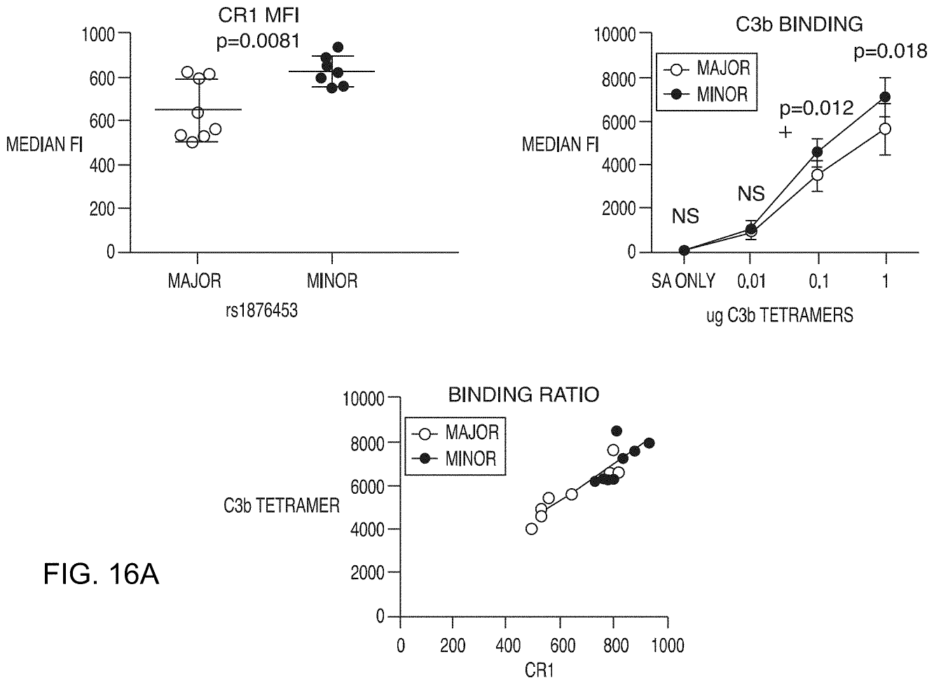

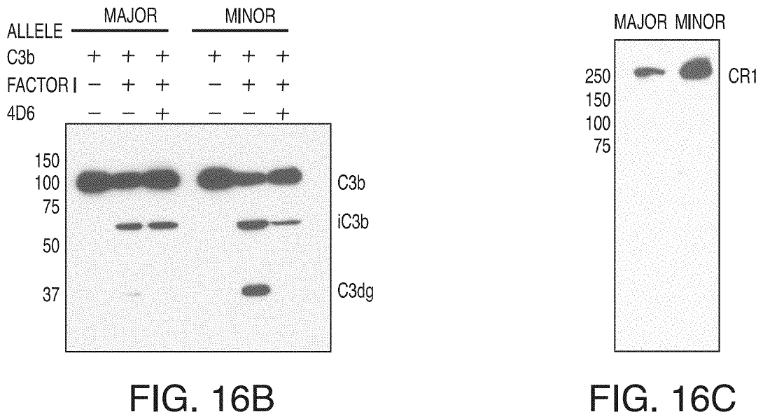

B cells from individuals with the protective CR2 SNP were found to bind more biot-C3b tetramers, with binding directly correlated with CR1 expression. FIG. 16A. Likewise, B cell lysates from an individual homozygous for the protective minor allele processed more C3b to C3d in 30 minutes than an individual homozygous for the major allele. FIG. 16B. This observation is likely related to the higher levels of CR1 present in the B cell lysates from this individual. FIG. 16C. These data show that the increase in B cell CR1 associated with the protective SNP is functionally relevant with respect to the binding and processing of C3b-ligands.

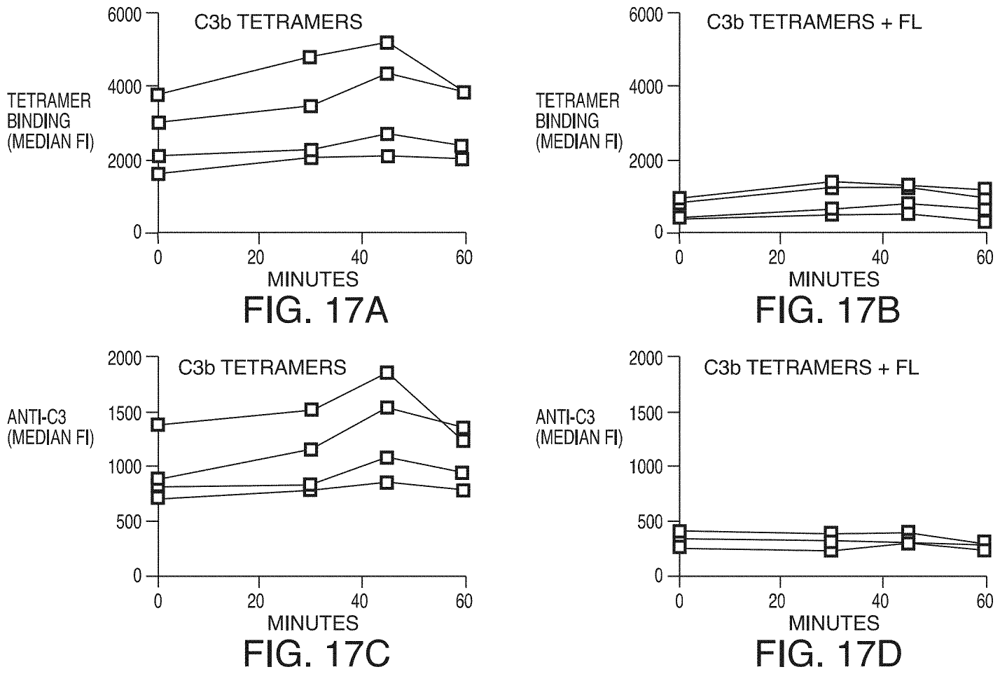

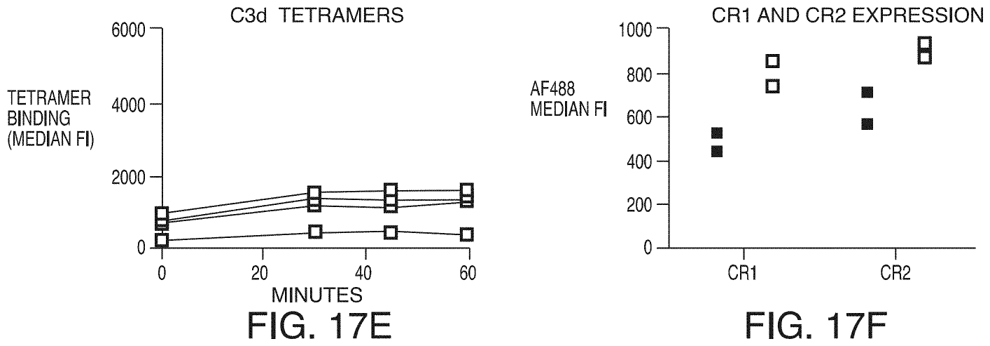

Binding of C3b tetramers to intact B cells occurred at levels proportional to the expression of CR1 and peaked at 45 minutes, with more marked increases in binding seen in subjects with higher levels of CR1 (38.4-44.2% vs 25.5-30.2%). FIG. 17F and FIG. 17A, respectively. This binding required CR1, as preincubation with a blocking antibody to CR1 (3D9) completely eliminated C3b tetramer binding (data not shown). More C3b tetramers bound to B cells (FIG. 17A) than C3dg tetramers generated using recombinant biotinylated C3dg (FIG. 17E), likely because of the multiple C3b binding domains present in each CR1 molecule. Henson et al., 2001. “Generation of recombinant human C3dg tetramers for the analysis of CD21 binding and function. J Immunol Methods 258: 97-109; Klickstein et al., 1987. “Human C3b/C4b receptor (CR1): demonstration of long homologous repeating domains that are composed of the short consensus repeats characteristic of C3/C4 binding proteins” J Exp Med 165: 1095-112; FIG. 17A; and FIG. 17E. When factor I was added to the samples, levels of tetramers bound to the cells decreased immediately (FIG. 17B) as did binding of anti-C3 (difference between FIGS. 17C and 17D), consistent with the rapid cleavage of C3b to iC3b by factor I. Newman et al., 1985. “Deposition of C3b and iC3b onto particulate activators of the human complement system. Quantitation with monoclonal antibodies to human C3” J Exp Med 161: 1414-1431. Cells from individuals with the protective SNP that expressed higher levels of CR1 (FIG. 17F) retained more tetramers (FIG. 17B), and this remained stable over time. The cleavage of C3b to iC3b lowers its affinity for CR1 by 100-fold and increases its affinity for CR2 by 10-fold, and the reduced binding of processed tetramers likely reflects release of some tetramers as well as constraint of tetramer binding to a single site on CR2 versus multiple sites on CR1. Ross et al., 1983. “Generation of three different fragments of bound C3 with purified factor I or serum. II. Location of binding sites in the C3 fragments for factors B and H, complement receptors, and bovine conglutinin” J Exp Med 158:334-352; and Alcorlo et al., 2011. “Unique structure of iC3b resolved at a resolution of 24 A by 3D electron microscopy” Proc Natl Acad Sci USA 108: 13236-13240.

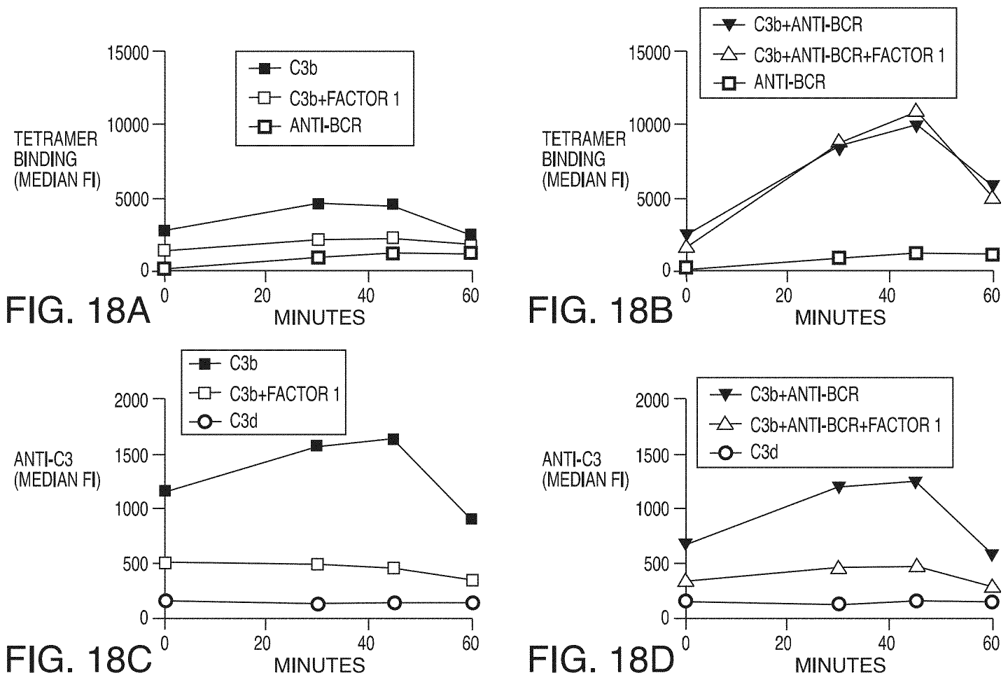

When the tetramers could engage the BCR as well as CR1 and/or CR2, binding was markedly enhanced and cleavage of C3b by factor I did not result in release of tetramers from the cells. FIG. 18A and FIG. 18B. Of note, the levels of bound tetramers went down after 60 minutes consistent with internalization, a process that we and others have shown involve CR1 and CR2 and can augment antigen presentation. Thornton et al., 1994. “Natural antibody and complement-mediated antigen processing and presentation by B lymphocytes” J. Immunol. 152: 1727-1737; Boackle et al., 1997. “CD21 augments antigen presentation in immune individuals” Eur. J. Immunol. 27:122-130; Boackle et al., 1998. “Complement opsonization is required for the presentation of immune complexes by resting peripheral blood B cells” J. Immunol. 161: 6537-6543; Grattone et al., 1999. “Co-operation between human CR1 (CD35) and CR2 (CD21) in internalization of their C3b and iC3b ligands by murine-transfected fibroblasts” Immunology 98:152-157. These data suggest that complement-opsonized complexes may preferentially remain bound to and modify the function of antigen-specific B cells, and that this might be augmented in individuals with the protective SNP who express higher levels of CR1 on their B cells.

II. Complement Receptor 2 (CR2)

The Complement Receptor 2 (CR2) gene is located directly 5′ of the Complement Receptor 1 (CR1) gene at chromosome locus 1q32, and the expression of these two genes is coregulated in human B cells. B cells from individuals with a protective SNP (e.g., r51876453) had increased transcription of the CR1 gene but no changes were observed in CR2 gene transcriptional levels. The rs1876453 SNP lies within a putative B cell enhancer, and several potential mechanisms suggest that the rs1876453 SNP could alter the transcription of CR1 independently of CR2.

Additionally, B cells express higher levels of CR1 that bind and process more C3b-tetramers. Consequently, the subsequent retention of processed tetramers on the B cell membrane is influenced by the antigen specificity of the B cell. Mice deficient in both CR1 and CR2 are unable to delay progression to autoimmune diabetes when exposed to inflammatory stimuli and that this is associated with impaired induction of regulatory B cells. Therefore, the data presented herein suggest that B cell CR1 plays a role in the maintenance of antigen-specific B cell tolerance under the inflammatory conditions in which autoimmunity arises.

The CR2/CD21 promoter requirements for CR2 receptor expression was discussed in the context of observing reduced CR2 receptor expression in lupus patients but did not report on CR1 receptor levels. Taylor et al., “Focused transcription from the human CR2/CD21 core promoter is regulated by synergistic activity of TATA and Initiator elements in mature B cells.” Cellular & Molecular Immunology 13:119-131 (2015).

It has been reported that human CR2 (hCR2) prematurely expressed under a murine Vk2 promoter/Vk2-4 enhancer minigene during the CD43+CD25− late pro-B cell stage of development results in peripheral B cells with impaired responses to immunization with T-dependent antigens. hCR2 transgenic (Tg) mice demonstrate a severe defect in T-independent antigen responses and are substantially protected from clinical arthritis, synovitis and cartilage/bone destruction in a collagen induced arthritis model. This outcome was found despite the apparently normal development of autoreactive T cells with equivalent cytokine and proliferative responses to antigen when compared to non-Tg control mice. These data suggest the presence of an intrinsic B cell defect in the hCR2 Tg mice. It was also shown that an hCR2-dependent Ca′ influx can be generated in both developing and mature Tg B cells, but with different rates of decay as compared to control wild-type (WT) mice. In addition, although analysis of tyrosine-phosphorylated proteins in WT and Tg B cells following B cell receptor (BCR)-induced activation revealed the presence of distinctly different phosphorylation patterns, no differences were identified in several candidate protein targets. Overall, these data suggest that premature hCR2 expression and the consequences thereof during B cell development intrinsically alters the way mature B cells develop and subsequently respond to antigen through the BCR signaling complex. Kulik et al., “Intrinsic B cell hypo-responsiveness in mice prematurely expressing human CR2/CD21 during B cell development” Eur. J. Immunol. 37: 623-633 (2007).

A functional variant in the gene for complement receptor 2 (CR2/CD21) has been identified as a possible explanation for a significantly decreased risk of systemic lupus erythematosus (SLE) in some individuals. The CR2/CD21 allele also increased B cell expression of complement receptor 1 (CR1), which was suggested to play an inhibitory role in B-cell activation, suggesting SLE susceptibility. Maria Gifford: “Found: Why B Cells Attack DNA in Lupus.” News|Lupus, Musculoskeletal Citations. rheumatologynetwork.com/lupus/found-why-b-cells-attack-dna-lupus. Sep. 12, 2014.

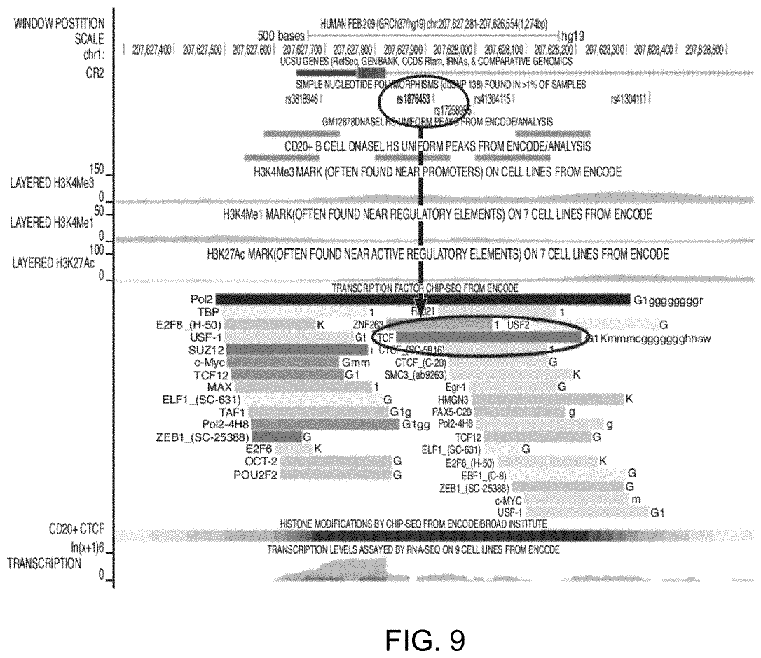

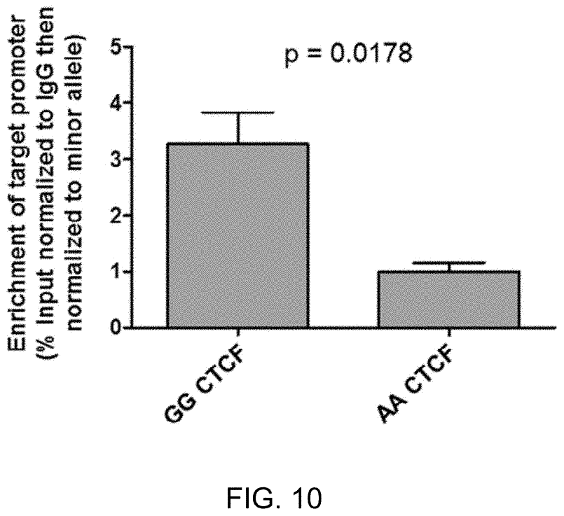

A rs1876453 chromosome location (CR2 intron 1) was reported to be associated with SLE, specifically when subjects with lupus were stratified based on the presence of dsDNA autoantibodies. Although allele-specific effects of the rs1876453 chromosome location on B cell CR2 mRNA or protein levels were not identified, levels of complement receptor 1 (CR1/CD35) mRNA and protein were significantly higher on the B cells of subjects harboring the minor allele. It was concluded that the CR2 rs1876453 chromosome location may have long-range effects on gene regulation that decrease susceptibility to lupus. Zhao et al., “An Intronic CR2 Polymorphism Associated With Systemic Lupus Erythematosus Alters CTCF Binding and CR1 Expression” Arthritis Rheum 65 Suppl 10:2703 (2013). See FIG. 9.

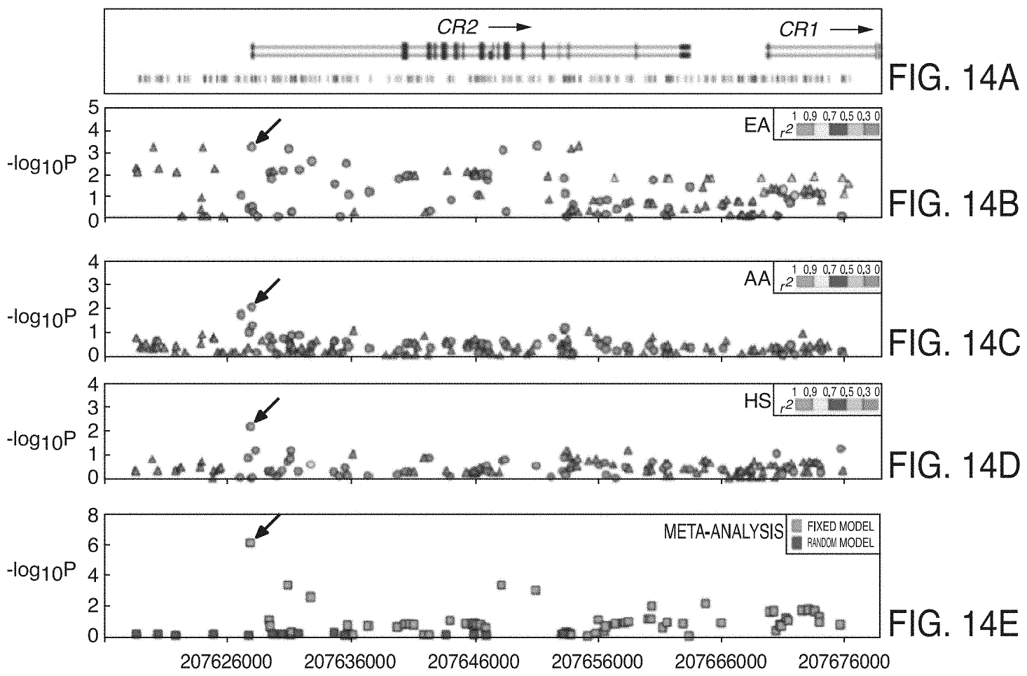

The CR2 gene was identified as a candidate gene for lupus susceptibility in the NZM2410 mouse model of SLE based on structural and functional alterations in its protein products. Boackle et al., 2001. “Cr2, a candidate gene in the murine Sle1c lupus susceptibility locus, encodes a dysfunctional protein. Immunity 15:775-785. This led to an examination of a role for CR2 in human lupus. An association of a common three single-nucleotide polymorphism (SNP) CR2 haplotype was found in Caucasian and Chinese lupus simplex families with a 1.54-fold increased risk for disease development, and these findings were confirmed in a case-control analysis of an independent European-derived population. Wu et al., 2007. “Association of a common complement receptor 2 haplotype with increased risk of systemic lupus erythematosus’ Proc Natl Acad Sci USA 104:3961-3966; and Douglas et al., 2009. “Complement receptor 2 polymorphisms associated with systemic lupus erythematosus modulate alternative splicing” Genes Immunol 10: 457-469. In the latter study, a minor allele haplotype was identified that was associated with decreased risk of lupus. This allele was subsequently fine-mapped to a region spanning CR2 in 15,750 subjects from four ancestral groups to identify a potential causal variant for this protective effect. The strongest association signal was detected at rs1876453 in intron 1 of CR2 (Pₘₑₜₐ=4.2×10⁻⁴, OR 0.85), specifically when subjects were stratified based on the presence of dsDNA autoantibodies (case-control Pₘₑₜₐ=7.6×10⁻⁷, OR 0.71; case-only Pₘₑₜₐ=1.9×10⁴, OR 0.75). Zhao et al., 2016. “Preferential association of a functional variant in complement receptor 2 with antibodies to double-stranded DNA” Ann Rheum Dis 75: 242-252; and FIGS. 14A-14E. Since autoantibodies to dsDNA correlate with disease onset, activity, and severity, these data suggested that the SNP modifies regulatory mechanisms that restore B cell tolerance to a relevant lupus autoantigen and as a disease-protective allele might prove a good target for therapy in early or preclinical disease.

III. Long NonCoding Ribonucleic Acids (lncRNA)

LncRNAs are the largest class of noncoding RNAs, comprising over 20,000 genes annotated in the ENcylopedia of DNA Elements (ENCODE) and other reference databases. They are defined as transcripts produced by RNA polymerase II that are longer than 200 nucleotides and devoid of an open reading frame that can be translated into a protein. Derrien et al., 2012. “The GENCODE v7 catalog of human long noncoding RNAs: analysis of their gene structure, evolution, and expression. Genome Res 22: 1775-1789.

The function of most lncRNAs is unknown, but they have been proposed to play roles in both negatively and positively regulating gene expression. They can regulate expression of protein-coding genes at both the transcriptional and posttranscriptional levels. Posttranscriptional regulation could occur by competing with endogenous RNA to regulate microRNA levels, modulating mRNA stability and translation by homologous base pairing, or altering cellular localization of mRNAs. Transcriptional regulation can occur in cis with their effects restricted to the chromosome from which they are transcribed and in trans with their effects targeting gene transcription on other chromosomes. Both cis- and trans-acting lncRNAs can mediate their effects through their RNA transcripts; cis-acting lncRNAs can also regulate gene transcription as a result of the process of splicing or of transcription itself.

LncRNAs have been implicated in the regulation of tissue and developmental stage-specific transcription of genes. Mutations and dysregulation of lncRNAs have been increasingly linked with diverse human diseases. Wapinski et al., 2011. “Long noncoding RNAs and human disease” Trends Cell Biol 21: 354-361. Despite their pervasive transcription, very little is currently known about the regulation and function of lncRNAs. They have been proposed to play roles in both negatively and positively regulating gene expression. Their functions are determined by their secondary and tertiary structures and they do not share sequence homology with their targets. Recent reports have demonstrated a role for lncRNA in the regulation of Toll-like-receptor mediated immune responses and differentiation and function of human dendritic cells and T cells, implicating them in immune mechanisms that could modulate autoimmune disease. Mauger et al., 2013. “The genetic code as expressed through relationships between mRNA structure and protein function” FEBS Lett 587: 1180-1188; Carpenter et al., 2013. “A long noncoding RNA mediates both activation and repression of immune response genes” Science 341:789-792; Wang et al., 2014. “The STAT3-binding long noncoding RNA lnc-DC controls human dendritic cell differentiation” Science 344: 310-313; Hu et al., 2013. “Expression and regulation of intergenic long noncoding RNAs during T cell development and differentiation” Nat Immunol 14: 1190-1198; Spurlock et al., 2015. “Expression and functions of long noncoding RNAs during human T helper cell differentiation” Nat Commun 6: 6932; and Collier et al., 2012. “Cutting edge: influence of Tmevpg1, a long intergenic noncoding RNA, on the expression of Ifng by Th1 cells” J Immunol 189: 2084-2088.

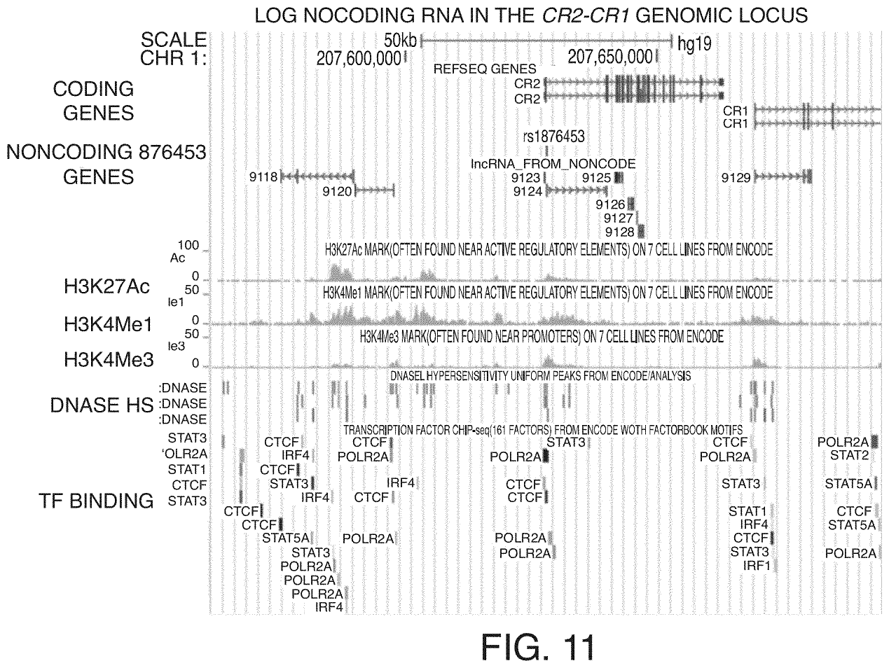

Recent studies suggest that the majority of the human genome is transcribed, but only about 2% accounts for protein-coding exons. Long noncoding RNAs (lncRNAs) constitute a heterogenic class of RNAs that includes, for example, intergenic lncRNAs, antisense transcripts, and enhancer RNAs. Moreover, alternative splicing can lead to the formation of circular RNAs. In support of putative functions, GWAS for cardiovascular diseases have shown predictive single-nucleotide polymorphisms in lncRNAs, such as the 9p21 susceptibility locus that encodes the lncRNA antisense noncoding RNA in the INK4 locus (ANRIL). Many lncRNAs are regulated during disease. For example, metastasis-associated lung adenocarcinoma transcript 1 (MALAT1) and myocardial infarction-associated transcript (MIAT) were shown to affect endothelial cell functions and diabetic retinopathy, whereas lncRNA-p21 controls neointima formation. In the heart, several lncRNAs were shown to act as microRNA sponges and to control ischemia-reperfusion injury or act as epigenetic regulators. Boon et al., “Long Noncoding RNAs: From Clinical Genetics to Therapeutic Targets?” J Am Coll Cardiol. 15; 67(10):1214-1126 (2016); and FIG. 11.

A long non-coding RNA NeST expressed by a lentivirus vector for treatment of inflammatory conditions, autoimmune diseases—including lupus erythematosus, infectious diseases, immunodeficiency, and cancer has been reported. A vaccinia-based infection/transfection system is described to provide inducible and transient cytoplasmic expression. A specific use of NeST was described to control levels of interferon-gamma (IFN-γ) production by leukocytes. There was no specific mention of complement receptors, CD21, CD35, or B cells. Kirkegaard, et al. “Immunomodulation By Controlling Interferon-Gamma Levels With The Long Non-Coding RNA NeST” United States Patent Application Publication Number 2014/0056929 (2014).

Lentiviral vectors have been reported for expressing a lncRNA induced gene expression system in a human cell. Induction or enhanced activation of a specific gene was mediated by a small double-stranded RNA (dsRNA) complementary to an antisense lncRNA from the promoter region. The lncRNA transcriptional gene activation was shut off by triggering a cre-recombination event with a SV40-poly(A) signal that positioned into a reverse orientation to block transcription. In order to provide the reverse orientation capability, the SV40-poly(A) signal was flanked by two inverted LoxP sites. Zhang, et al., “The role of antisense long noncoding RNA in small RNA-triggered gene activation.” RNA 20(12):1916-1928 (2014). A lentiviral vector may also be controlled using a tetracycline transcriptional activation system (TetR system) for expressing a gene. In these systems, low expression levels of the gene result when the system is turned “off” while high levels of gene expression occur in the “on”-state. Loew, et al., “Improved Tet-responsive promoters with minimized background expression” BMC Biotechnology 10:81 (2010).

A sustained, targeted, high-level transgene expression in primary B lymphocytes may be useful for gene therapy in B cell disorders. Several candidate B-lineage predominant self-inactivating lentiviral vectors (LV) containing alternative enhancer/promoter elements were developed including: the immunoglobulin β (Igβ) (B29) promoter combined with the immunoglobulin μ enhancer (EμB29); and the endogenous BTK promoter with or without Eμ (EμBtkp or Btkp). LV-driven enhanced green fluorescent protein (eGFP) reporter expression was evaluated in cell lines and primary cells derived from human or murine hematopoietic stem cells (HSC). In murine primary cells, EμB29 and EμBtkp LV-mediated high-level expression in immature and mature B cells compared with all other lineages. Expression increased with B cell maturation and was maintained in peripheral subsets. Expression in T and myeloid cells was much lower in percentage and intensity. Similarly, both EμB29 and EμBtkp LV exhibited high-level activity in human primary B cells. In contrast to EμB29, Btkp and EμBtkp LV also exhibited modest activity in myeloid cells, consistent with the expression profile of endogenous Bruton's tyrosine kinase (Btk). Notably, EμB29 and EμBtkp activity was superior in all expression models to an alternative, B-lineage targeted vector containing the EμS.CD19 enhancer/promoter. In summary, EμB29 and EμBtkp LV comprise efficient delivery platforms for gene expression in B-lineage cells. Sather et al., “Development of B-lineage Predominant Lentiviral Vectors for Use in Genetic Therapies for B Cell Disorders” Molecular Therapy vol. 19(3):515-525 (2011).

LncRNAs can be generated through pathways similar to those of protein-coding genes, with similar histone-modification profiles, splicing signals, and exon/intron lengths. In contrast to protein-coding genes, they are biased towards two-exon transcripts, predominantly localized in the chromatin and nucleus, and generally expressed at lower levels; they also display more tissue-specific expression patterns. Derrien et al., 2012. “The GENCODE v7 catalog of human long noncoding RNAs: analysis of their gene structure, evolution, and expression” Genome Res 22: 1775-1789.

In order to better understand the function of the spliced noncoding RNA transcript that is present at higher levels in individuals with the protective SNP, knowledge of the full extent of the transcript should be obtained, where it is located within the cell, whether it is restricted to B cells or has a broader tissue distribution, and how it responds to activating cell signals. Knowledge of its intracellular location and tissue distribution will shed light on its function, whereas understanding how it responds to cell signals will indicate how it is regulated. Finally, with knowledge of the extent of the transcript, we can generate tiling oligonucleotides to map its localization on the genome using Chromatin Isolation by RNA Purification (ChIRP) followed by next-generation sequencing. ChIRP followed by mass spectrometry may be used to identify the proteins with which it is associated and therefore generate testable hypotheses about its functions.

One mode by which lncRNAs act is in the recruitment of epigenetic protein factors for regulation of chromatin states. They also play activating roles in the regulation of gene transcription primarily by recruiting activating proteins and protein complexes but also by mediating chromatin interactions and evicting repressive machineries. Kornienko et al., 2013. “Gene regulation by the act of long non-coding RNA transcription” BMC Biol 11: 59; Lam et al., 2014. “Enhancer RNAs and regulated transcriptional programs” Trends Biochem Sci 39: 170-182; Rinn et al., 2012. “Genome regulation by long noncoding RNAs” Annu Rev Biochem 81: 145-166; Wang et al., 2011. “Molecular mechanisms of long noncoding RNAs.’ Mol Cell 43:904-914; and Zhang et al., 2014. “Long noncoding RNA-mediated intrachromosomal interactions promote imprinting at the Kcnq1 locus” J Cell Biol 204: 61-75. The data presented herein examines protein partners of a CR2 lncRNA to identify methods to imitate or enhance its function.

In one embodiment, the present invention contemplates a composition comprising a lncRNA and a splice inducer. In one embodiment, the lncRNA is an unspliced lncRNA. In one embodiment, the lncRNA is spliced lncRNA. Although it is not necessary to understand the mechanism of an invention it is believed that the splice inducer is present to create lncRNA splice variants. Targeting of pre-mRNA by short splice-switching oligonucleotides (SSOs) is increasingly being used as a therapeutic modality, one rationale being to disrupt splicing so as to remove exons containing premature termination codons, or to restore the translation reading frame around out-of-frame deletion mutations. Investigations into the effect of chemically linking individual SSOs ascertained equimolar cellular uptake that would provide for more defined drug formulations. In contrast to conventional bispecific SSOs generated by conjugation in solution, a protocol for synthesis of bispecific SSOs on solid phase was outlined. These SSOs comprised of either a non-cleavable hydrocarbon linker or disulfide-based cleavable linkers. To assess the efficacy of these SSOs splice switching was utilized to bypass a disease-causing mutation in the DMD gene concurrent with disruption of the reading frame of the myostatin gene (Mstn). The premise of this approach is that disruption of myostatin expression is known to induce muscle hypertrophy and so for Duchenne muscular dystrophy (DMD) could be expected to have a better outcome than dystrophin restoration alone. All tested SSOs mediated simultaneous robust exon removal from mature Dmd and Mstn transcripts in myotubes. The results also demonstrated that using cleavable SSOs is preferred over the non-cleavable counterparts and that these are equally efficient at inducing exon skipping as cocktails of monospecific versions. A protocol was developed for solid-phase synthesis of single molecule cleavable bispecific SSOs that can be efficiently exploited for targeting of multiple RNA transcripts. Bestas et al., “Design and application of bispecific splice-switching oligonucleotides” Nucleic Acid Ther. 24(1):13-24 (2014).

IV. Regulatory B Cells and Autoimmune Diseases

Both genetic and environmental factors influence the development of autoimmune diseases, which result from an imbalance between activation and regulation of the immune system. Transancestral mapping techniques recently identified a single nucleotide polymorphism (SNP), rs1876453, located just inside the first intron of the immune-associated complement receptor 2 (CR2) gene that was enriched in controls rather than lupus cases, suggesting that it had a protective effect. This effect was most prominent when patients tested positive for autoantibodies to double-stranded DNA (dsDNA), which correlate with disease onset, activity, and severity, suggesting that the rs1876453 SNP sequence modifies regulatory mechanisms that restore B cell tolerance to a relevant lupus autoantigen.

Autoimmunity and inflammation may be controlled in part by regulatory B cells, including a recently identified IL-10-competent CD1d^{high}CD5⁺, a B cell subset termed B10 cells that represents 1-3% of adult mouse spleen B cells. Pathways that influence B10 cell generation and IL-10 production have been identified and compared with previously described regulatory B cells. For example, IL-10-competent B cells were predominantly CD1d^{high}CD5⁺ in adult spleen and were a prevalent source of IL-10, but not other cytokines. B10 cell development and/or maturation in vivo required Ag receptor diversity and intact signaling pathways, but not T cells, gut-associated flora, or environmental pathogens. Spleen B10 cell frequencies were significantly expanded in aged mice and mice predisposed to autoimmunity, but were significantly decreased in mouse strains that are susceptible to exogenous autoantigen-induced autoimmunity. LPS, PMA, plus ionomycin stimulation in vitro for 5 h induced B10 cells to express cytoplasmic IL-10. However, prolonged LPS or CD40 stimulation (48 h) induced additional adult spleen CD1d^{high}CD5⁺ B cells to express IL-10 following PMA plus ionomycin stimulation. Prolonged LPS or CD40 stimulation of newborn spleen and adult blood or lymph node CD1d^{low} and/or CD5⁻ B cells also induced cytoplasmic IL-10 competence in rare B cells, with CD40 ligation uniformly inducing CD5 expression. IL-10 secretion was induced by LPS signaling through MyD88-dependent pathways, but not following CD40 ligation. LPS stimulation also induced rapid B10 cell clonal expansion when compared with other spleen B cells. Thereby, both adaptive and innate signals regulate B10 cell development, maturation, CD5 expression, and competence for IL-10 production. Yanaba et al., “The Development and Function of Regulatory B Cells Expressing IL-10 (B10 Cells) Requires Antigen Receptor Diversity and TLR Signals” Journal of Immunology 182:7459-7472 (2009).

Most disease-associated genetic variants lie outside protein-coding regions and are highly enriched in enhancers. Enhancers are DNA sequences that recruit transcription factors to regulate the transcription of target genes in a cell type-specific manner. They were originally defined as regulatory elements that, regardless of orientation, position, and distance, can enhance the expression of a gene. Maurano et al., 2012. “Systematic localization of common disease-associated variation in regulatory DNA. Science 337: 1190-1195; Andersson et al., 2014. “An atlas of active enhancers across human cell types and tissues” Nature 507: 455-461; and Banerji et al., 1981. “Expression of a beta-globin gene is enhanced by remote SV40 DNA sequences” Cell 27: 299-308.

Enhancers act as platforms for the binding of integrated cell-type specific transcription factors, and they can be located many thousands of kilobases away from their target genes. The presence of transcription factors at enhancers is associated with regions of low nucleosome occupancy that are hypersensitive to DNA nucleases, and the ability of enhancer-bound transcription factors to activate transcription is dependent on the recruitment of coactivator proteins.

Nucleosomes marked with histone H3 lysine 27 acetylation (H3K27ac) and histone H3 lysine 4 dimethylation (H3K4me2) flank transcription factor and coactivator-occupied enhancer regions and distinguish active enhancers from poised enhancers flanked by nucleosomes marked only with histone H3 lysine 4 monomethylation (H3K4me1). Active enhancers flanked by nucleosomes marked with H3K27Ac recruit RNA polymerase II (Pol II), leading to the production of enhancer-originating, short bidirectional RNAs of ˜500 nucleotides, termed eRNAs. In addition to serving as binding platforms for cell-type specific transcription factors, enhancers are occupied by DNA-binding effectors of cellular signaling pathways, and they give these signals cell-type specificity. Input from multiple enhancers can be integrated at a single promoter and likewise, information from a single enhancer can be conveyed to multiple promoters for coordinated gene regulation. He et al., 2010. “Nucleosome dynamics define transcriptional enhancers. Nat Genet 42: 343-347; Wang et al., 2014. “H3K4me2 reliably defines transcription factor binding regions in different cells” Genomics 103: 222-228; and Pekowska et al., 2010. “A unique H3K4me2 profile marks tissue specific gene regulation” Genome Res 20: 1493-1502.

Enhancer transcription correlates with enhancer activity (28) and is the earliest event in successive waves of coordinated transcription during cellular activation (29). Increasing numbers of reports suggest that this transcriptional activity is essential for enhancer function. These functions may be eRNA-independent and reflect the transcriptional process, specifically the movement across the chromatin template of Pol II and its associated enzymatic activities such as histone acetyltransferases and chromatin remodeling complexes. Alternatively, the eRNA may have specific cis- or trans-effects including the recruitment of protein complexes such as chromatin remodelers to the site of synthesis or the binding and targeting of effector proteins or protein complexes to distant sites brought nearby chromatin looping. Wang et al., 2011.

“Reprogramming transcription by distinct classes of enhancers functionally defined by eRNA” Nature 474: 390-394; Arner et al., 2015. “Gene regulation. Transcribed enhancers lead waves of coordinated transcription in transitioning mammalian cells” Science 347:1010-1014; Natoli et al., 2012. “Noncoding transcription at enhancers: general principles and functional models” Annu Rev Genet 46: 1-19; and Lam et al., 2014. “Enhancer RNAs and regulated transcriptional programs” Trends Biochem Sci 39: 170-182.

A. SLE Clinical Overview

Systemic lupus erythematosus (SLE) in humans is a complex autoimmune disease with a strong CR2 genetic component that determines a risk of developing circulating autoantibodies that recognize dsDNA. It has been observed that CR1 and CR2 levels are decreased by ˜50% in patients with systemic lupus erythematosus (SLE) and in mouse models. A decrease in CR2 is associated with a decrease in CR1. Autoreactive B cells are believed to play a role in SLE pathogenesis where there is a loss of tolerance to nuclear antigens, such as dsDNA. Conventional treatments usually are provided by global immunosuppression techniques that have the disadvantages of being toxic, requiring ongoing treatment, and often not being effective.

Like other autoimmune diseases, lupus is manifested by a long preclinical period during which autoantibodies are present in the absence of symptoms. As the time of diagnosis nears, the number and titers of autoantibodies increases and more specific autoantibodies for lupus appear, such as anti-dsDNA antibodies. During this preclinical period, it has been proposed that there is a balance between regulatory and effector immune mechanisms, but that this balance is lost upon disease onset. This same loss of balance may later explain the intermittent disease flares that occur in lupus, which are often preceded by a surge in autoantibodies to dsDNA that is not associated with clinical symptoms for several months. Pan et al., 2014. “A surge in anti-dsDNA titer predicts a severe lupus flare within six months” Lupus 23: 293-298.

The biological events that occur as individuals with preclinical lupus proceed towards the development of clinical disease have not been precisely established. In preclinical lupus autoimmunity, antinuclear, anti-Ro, anti-La, and antiphospholipid antibodies were the first autoantibodies to develop in asymptomatic individuals who subsequently developed SLE. Anti-Sm and anti-nRNP were noted close to the time of diagnosis associated with the development of other clinical manifestations, and anti-dsDNA antibodies appeared sometime in-between. There is evidence of increased global B cell activation in lupus, and a number of cytokines have been identified in the blood of patients with active disease, including IFNα, TNFα, IL6, and BAFF (66) with the levels of multiple soluble inflammatory mediators increasing several weeks before a clinical flare (67). Elevated levels of soluble inflammatory mediators have also been identified in unaffected first-degree relatives of lupus patients (68). It is likely that the inflammatory milieu alters the effect of lupus-associated SNPs, as documented recently for a Crohn's disease-associated SNP (69). Jacob et al., 2011. “Cytokine disturbances in systemic lupus erythematosus” Arthritis Res Ther 2011: 228; Munroe et al., 2014. “Proinflammatory adaptive cytokine and shed tumor necrosis factor receptor levels are elevated preceding systemic lupus erythematosus disease flare” Arthritis Rheumatol 66: 1888-1899; Munroe et al., 2014. “Elevated levels of soluble inflammatory mediators and lupus-specific connective tissue disease questionnaire score discern unaffected first-degree relatives of lupus patients from unaffected individuals not related to lupus patients” Arthritis Rheum 66: S1256-S1269; and Lee et al., 2013. “Human SNP Links Differential Outcomes in Inflammatory and Infectious Disease to a FOXO3-Regulated Pathway” Cell 155: 57-69

Approximately 60% of likely causal autoimmune variants map to enhancer-like elements that respond to immune activation by increasing histone acetylation and transcribing non-coding RNAs, linking causal disease variants with high probability to context-specific immune enhancers. Farh et al., 2015. “Genetic and epigenetic fine mapping of causal autoimmune disease variants” Nature doi:10.1038/nature13835. Since enhancer RNAs are expressed at low levels, they require global run-on sequencing (GRO-Seq) in order to be detected. Therefore, B cell activation by immune complexes or exposure to lupus-associated cytokines either developing in vivo or induced ex vivo will likely reveal alterations in the B cell transcriptome that are relevant in disease development, and the effect of the protective SNP on these transcriptional events will identify targets for novel therapeutics as well as actionable biomarkers for the use and timing of these interventions.

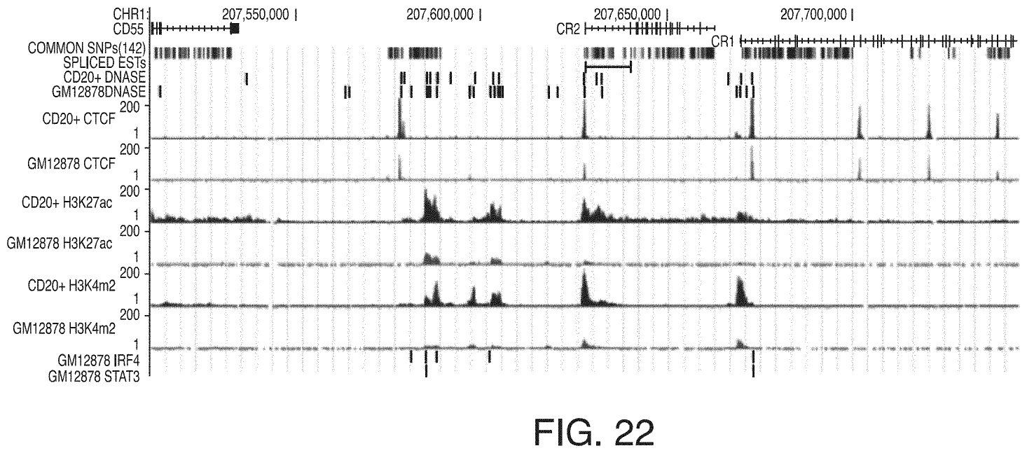

When pathogenic autoantibodies form immune complexes with self-antigen, they are opsonized by complement and are either cleared or cause persistent inflammation in tissues. We propose that the protective SNP increases B cell CR1 and primes other suppressive pathways so that when the cells encounter an excess of C3b-coated immune complexes, they develop a regulatory phenotype that allows them to suppress inflammation and arrest autoimmunity. Whether this regulatory phenotype results in the secretion of IL-10, the induction of regulatory T cells, and the suppression of effector T cells, as is seen with classical regulatory B cells, or whether a unique suppressive B cell develops is not known. For example, both IRF4 and STAT3 bind the enhancer regions upstream of CR2. FIG. 22. These transcription factors are induced by IL-21 and IL-35, which have been shown to expand regulatory B cells. Yoshizaki et al., 2012. “Regulatory B cells control T-cell autoimmunity through IL-21-dependent cognate interactions” Nature 491: 264-268; Wang et al., 2014. “Interleukin-35 induces regulatory B cells that suppress autoimmune disease” Nat Med 20: 633-641.

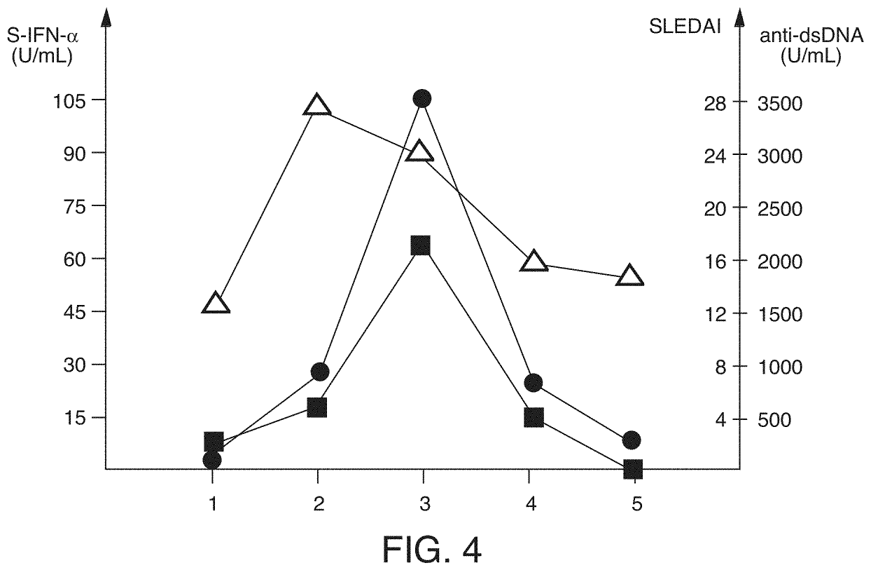

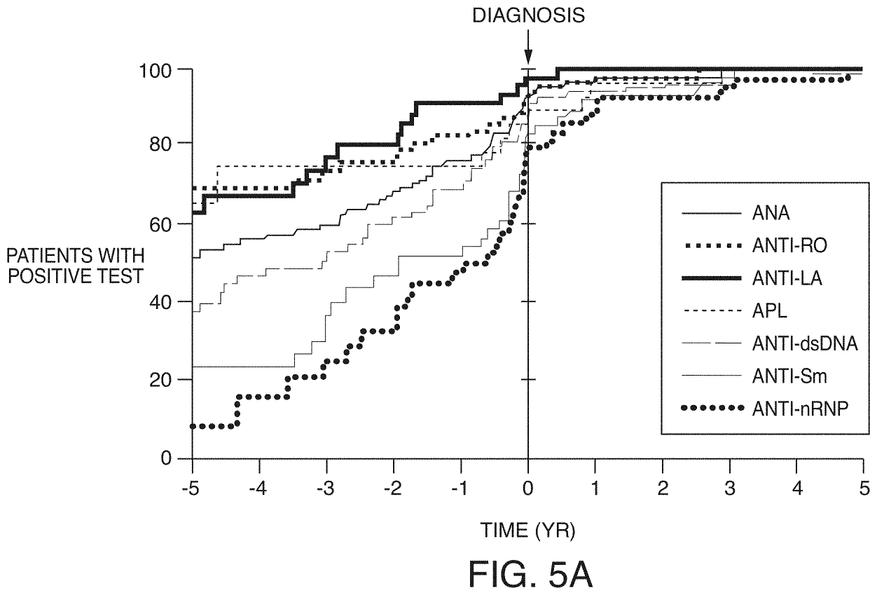

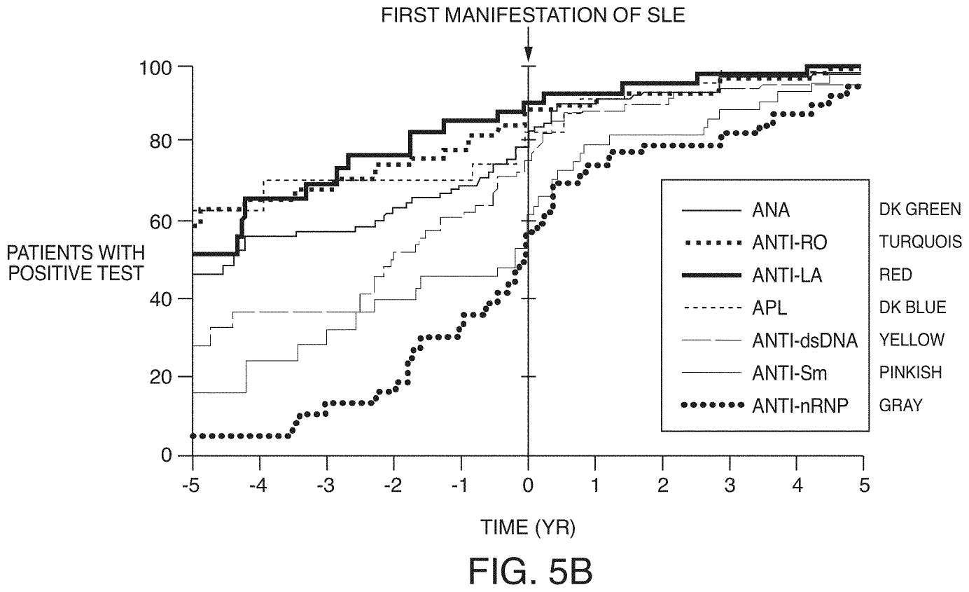

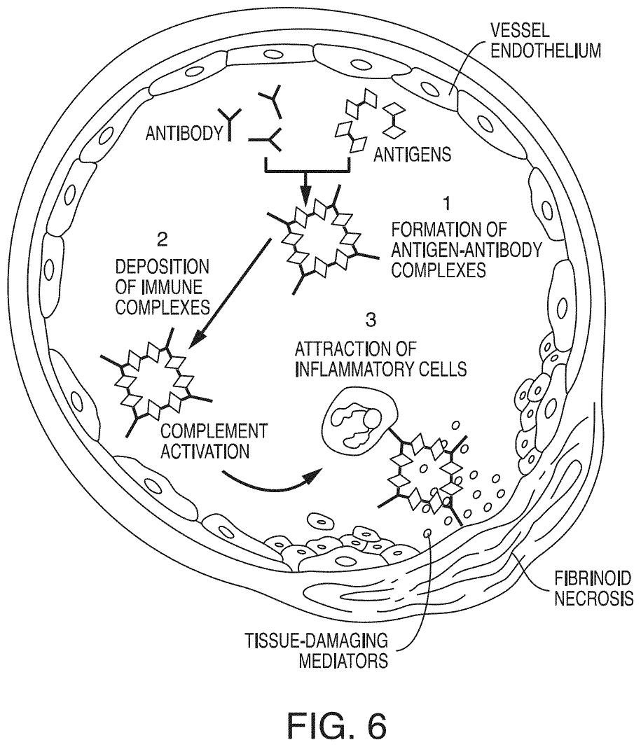

Systemic autoimmune disease is most prominent in young women and usually presents with symptoms including, but not limited to, rash, fatigue, arthritis to kidney failure, heart failure, stroke, or death. In some cases, permanent organ damage may result from autoimmune reactions. Ronnblom et al., Human Immunol 63:1181-1193 (2002); and FIG. 4. Autoantibody production usually precedes the symptomatic manifestations of SLE. See, FIGS. 5A-5B. Most models suggest that organ failure is a result of complement activation. See, FIG. 6.

One biomarker for SLE is the production of autoantibodies to nuclear antigens, and a positive test for antinuclear antibodies (ANA), indicating loss of tolerance to nuclear antigens, is found in ˜95% of patients with lupus. Low titer ANA are found in ˜30% of the “healthy” control population and may represent the initial manifestation of loss of tolerance in individuals with genetic risk factors for autoimmune disease, whereas asymptomatic individuals with higher titer ANA may have been exposed to environmental factors or possess additional susceptibility genes that induce a more penetrant form of autoimmunity. Tan et al., 1997. “Range of antinuclear antibodies in “healthy” individuals” Arthritis Rheum 40: 1601-1611.

Nonetheless, only ˜5% of healthy individuals with a positive ANA go on to develop lupus, and the factors that determine risk, which likely include interactions between variants of several genes in combination with environmental exposures, are not known. Loss of tolerance to nuclear antigens predates the development of clinically apparent disease in individuals with SLE, suggesting that a window of time exists during which intervention could prevent disease onset. Arbuckle et al., 2003. “Development of autoantibodies before the clinical onset of systemic lupus erythematosus. N Engl J Med 349:1526-1533.

Autoantibodies to double-stranded (ds)DNA, which are highly specific for lupus, are among the last autoantibodies to develop, and their induction may serve as a molecular switch that drives disease onset. Furthermore, since these antibodies are directly pathogenic and fluctuate with changes in disease activity, unlike other autoantibodies associated with systemic lupus erythematosus (SLE), their development can also determine the prognosis, or course that the disease will take following its development. The generation of multiple autoantibodies to chromatin and other self-antigens in lupus directly implicates B cells in lupus pathogenesis. These autoantibodies may develop as a result of genetically determined alterations in B cell function or in response to environmental stimuli such as increased autoantigen exposure, with both factors likely playing a role in disease development. Besides the production of autoantibodies, B cells can influence disease pathogenesis or prognosis by processing and presenting antigen to effector or regulatory T cells and by secreting cytokines that modulate disease.

Once lupus has developed, the B cell compartment is altered and B cells in general exhibit hyperactivity. Dorner et al., 2011. “Abnormalities of B cell subsets in patients with systemic lupus erythematosus” J Immunol Methods 363:187-197. Furthermore, therapies are often initiated that can mask the intrinsic alterations in B cell function that lead to disease. Healthy subjects can provide a cleaner background on which to study functional alterations caused by a specific variant, but they may lack other genetic variations that are necessary for full manifestation of the effects of the variant.

As described herein, first-degree relatives (FDR) of individuals with lupus are examined as a means of identifying relevant predictive and prognostic biomarkers of lupus based on allelic variation at rs1876453. These individuals would be expected to carry a higher burden of lupus susceptibility genes and may also have been exposed to environmental factors that would influence disease development. Therefore, such biomarkers would be strong candidates for further exploration as targets for new therapeutics. Such biomarkers may also indicate which individuals should receive these interventions and when, thereby providing an approach to prevent disease onset as well as disease flares.

B. SLE Genetic Causation

Genome-wide association studies have rapidly advanced our understanding of the genetic basis of systemic lupus erythematosus, with the identification of more than 40 robust susceptibility loci since 2008 using this approach. Cui et al., 2013. “Genetic susceptibility to SLE: recent progress from GWAS” J Autoimmun 41: 25-33. Interestingly, 70 of the 79 single-nucleotide polymorphisms (SNPs) cited in a recent review that achieved genome wide significance for association with lupus (p<5×10⁻⁸) were located in noncoding regions. This may be because the vast majority of the genome is intergenic and noncoding, with these SNPs only tagging the true risk variants for the observed associations. However, genome-wide surveys have shown that most of the human genome is actively transcribed, and the now established role of RNA as a major regulator of genetic networks in the cell suggests that SNPs in noncoding regions of the genome could have a significant impact on disease. Hangauer et al., 2013. “Pervasive transcription of the human genome produces thousands of previously unidentified long intergenic noncoding RNAs” PLoS Genet 9: e1003569; Djebali et al., 2012. “Landscape of transcription in human cells. Nature 489: 101-108; and Laederach A., 2007. “Informatics challenges in structured RNA” Brief Bioinform 8:294-303.

Although it is not necessary to understand the mechanism of an invention, it is believed that SLE patients may have an single nucleotide polymorphism (SNP) at chromosomal position rs1876453. This SNP is a G→A mutation and creates a cryptic splice acceptor site:
gcgagacggtGggg (SEQ ID. NO.12)→gcgagacggtAggg (SEQ ID. NO. 13)

This SNP may be within intron 1 of the CR2 allele, which is within or near transcription sites for long noncoding ribonucleic acids (lncRNAs). Intron 1 of CR2 rs1876453 carries: i) a major allele (G) that may be associated with an increased SLE risk; and ii) a minor allele (A) that may reduce SLE risk. This effect may be due, in part, to an increased B cell CR1 expression in the presence of highly specific Lupus dsDNA autoantibodies. It has been observed that CR1 mRNA levels and/or CR1/CD35 protein levels were significantly higher on B cells of subjects harboring the minor allele A. Minor allele A was also consistently associated with a decreased risk of serositis, renal disorder, haematological disorder and dsDNA autoantibodies in EA, AA and HS, of which the strongest association was detected with a decrease in dsDNA autoantibodies.

A rs1876453 minor allele associated with SLE patients having dsDNA autoantibodies was reported. Patients with the minor allele of rs1876453 showed levels of complement receptor 1 (CR1/CD35) mRNA and protein significantly higher on their B cells. Zhao, et al., “Preferential association of a functional variant in complement receptor 2 with antibodies to double-stranded DNA.” Ann Rheum Dis. 75:242-252, 2016-Published Online Sep. 1, 2014.

Chromosome position rs1876453 is generally considered a protective allele when present as an AA or GA haplotype. An association between an SNP rs1876453 major haplotype (G) and SLE was seen across several human ethnic groups with a stronger association in patients with detectable SLE dsDNA autoantibodies. Several transcriptional binding factors have been suggested to have binding sites in the area of rs1876453. However, while CR2 cell surface expression is the same for A and G alleles, CR1 expression on primary B cells is higher in the presence of the protective SNP rs1876453 A allele. Thus, SNP rs1876453 allele A may be associated with a decreased risk of developing SLE and dsDNA autoantibodies and associated with an increase in CR1 mRNA and protein levels in resting B cells. Zhao et al., “An Intronic CR2 Polymorphism Associated with Systemic Lupus Erythematosus Alters CTCF Binding and CR1 Expression.” Arthritis Rheum 65 Suppl 10:2703 (2013).

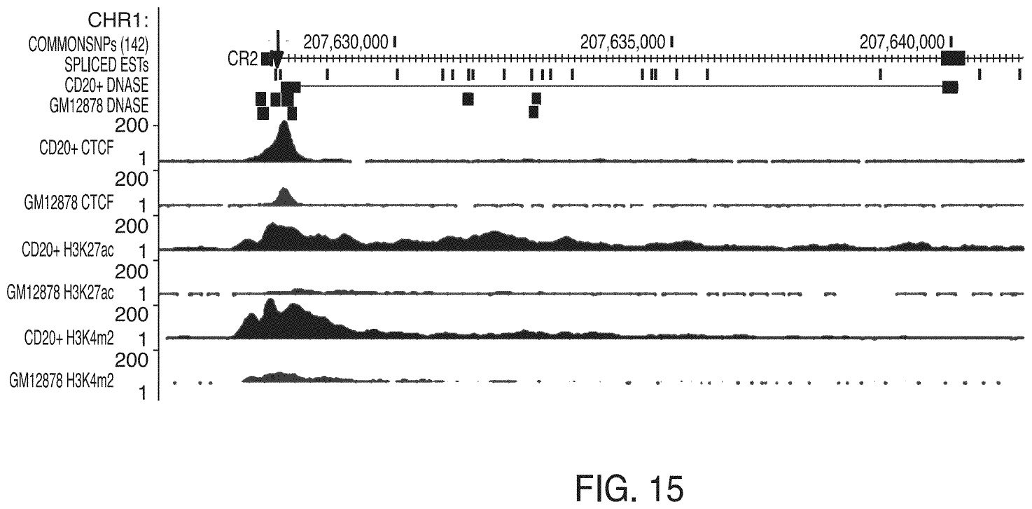

rs1876453 is located 97 nucleotides inside the first intron of CR2 within a putative active B cell enhancer based on the presence of open chromatin and characteristic histone marks. Rosenbloom et al., 2015. “The UCSC Genome Browser database 2015 update” Nucleic Acids Res 43: D670-681; and FIG. 15. rs1876453 has been shown to reduce the binding of several protein complexes, including one containing CCCTC-binding factor (CTCF) (16), suggesting that it alters the function of this domain. Zhao et al., 2016. “Preferential association of a functional variant in complement receptor 2 with antibodies to double-stranded DNA” Ann Rheum Dis 75:242-252. A possibility that the SNP could modify levels of an intragenic noncoding transcript was also explored. FIG. 15, Spliced EST track. The first exon of this transcript is located 78 nucleotides downstream of rs1876453 and contains 305 nucleotides of the first intron of CR2 that is spliced to an exon containing the first 231 nucleotides of the second exon of CR2.

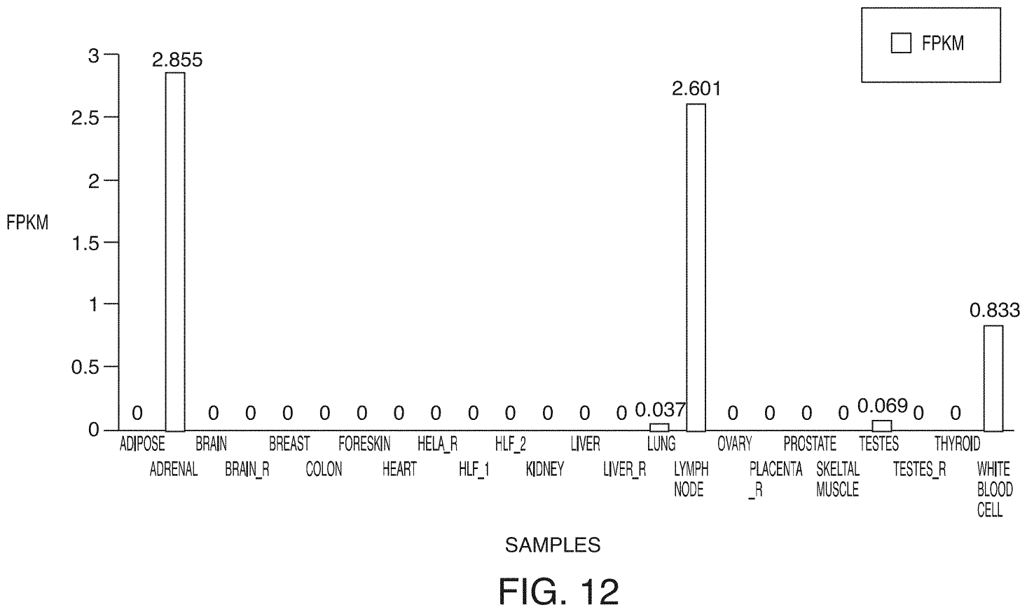

However, when rs1876453 is present as a GG haplotype, a patient may then be at risk for developing SLE. For example, primary B cells with an AA rs1876453 haplotype or a GA rs1876453 haplotype may show a significantly higher expression of CR1 in combination with higher levels of lncRNA 9124. On the other hand, primary B cells with a GG rs1876453 haplotype may show a significantly lower expression of CR1 in combination with lower levels of lncRNA 9124. The expression of lncRNA 9124 appears primarily specific to the adrenals, lymph nodes and white blood cells. FIG. 12.

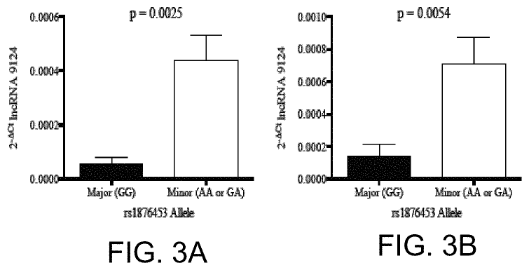

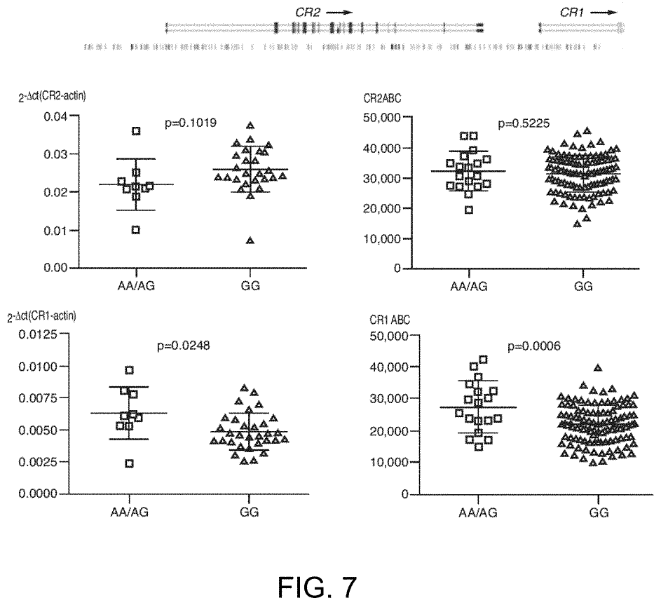

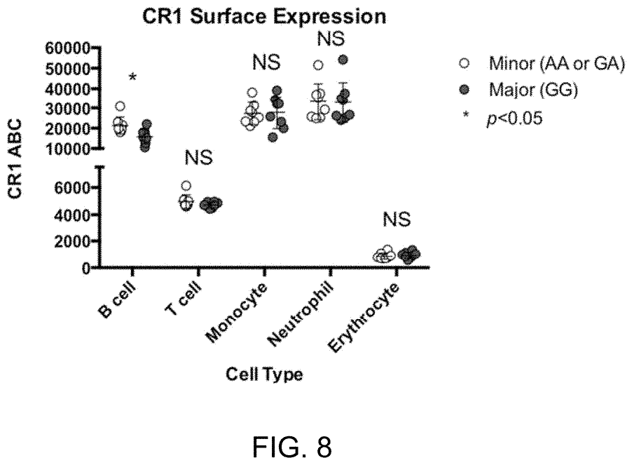

The data presented herein finds that subjects with the minor protective allele had three-fold higher levels of the transcript by quantitative rtPCR compared with those subjects with the major allele. FIGS. 3A-3B. Intragenic enhancers can act as alternative promoters for the generation of spliced multiexonic poly(A)+ RNAs that reflect the host gene's structure (18), and if the putative enhancer in which the protective CR2 SNP lies is activated as a result of the SNP, this could increase the levels of this transcript, which could have long range effects on gene transcription. Kowalczyk et al., 2012. “Intragenic enhancers act as alternative promoters” Mol Cell 45: 447-458. Alternatively, activation of the enhancer within which the protective SNP lies could have direct effects on neighboring or distant promoters for additional noncoding and coding genes that might modify B cell responses to self-antigens. Since CR2 is located 40 kB directly 5′ of CR1 at chromosome 1q32 and their expression is co-regulated in human B cells, we examined the effects of rs1876453 on the transcription of both genes. It was found that B cells from individuals with the protective SNP had increased transcription of the CR1 gene but no changes in CR2 transcriptional levels. This effect was specific for B cells as it was not observed in other CR1-expressing cells in the peripheral blood, including erythrocytes, monocytes, neutrophils and T cells, consistent with an effect of the SNP on enhancers and long noncoding RNA, which have cell-type specific effects. Zhao et al., 2016. “Preferential association of a functional variant in complement receptor 2 with antibodies to double-stranded DNA” Ann Rheum Dis 75:242-252; and FIG. 7 and FIG. 8, respectively.

The SNP lies within a putative B cell enhancer, and we showed that it altered the binding of several protein complexes, including one containing CCCTC binding factor (CTCF). In the intergenic region 5′ of CR2, there are additional putative B cell enhancer elements, and this region interacts with the CR1 promoter by Hi-C. CTCF, a highly conserved and ubiquitously expressed zinc finger protein, is a master regulator of genome spatial organization that mediates chromatin loops within the genome that influence proper gene expression through cross-talk between promoters and enhancers. It also binds to insulator elements to prevent the spread of heterochromatin and to restrict transcriptional enhancers from promiscuously activating unintended promoters and can act as a transcriptional activator or repressor. Handoko et al., 2011. “CTCF-mediated functional chromatin interactome in pluripotent cells” Nat Genet 43: 630-638; Bell et al., 1999. “The protein CTCF is required for the enhancer blocking activity of vertebrate insulators” Cell 98: 387-396; and Kim et al., 2015. “CTCF as a multifunctional protein in genome regulation and gene expression” Exp Mol Med 47: e166.

Variants that disrupt CTCF-associated boundary domains can rewire long-range regulatory architecture and cause unexpected promoter-enhancer interactions. One hypothesis for the effect of the protective CR2 SNP on CR1 transcription is that reduced CTCF binding has allowed the putative intragenic CR2 enhancer access to the CR1 promoter. Lupianez et al., 2015. “Disruptions of topological chromatin domains cause pathogenic rewiring of gene-enhancer interactions” Cell 161: 1012-1025.

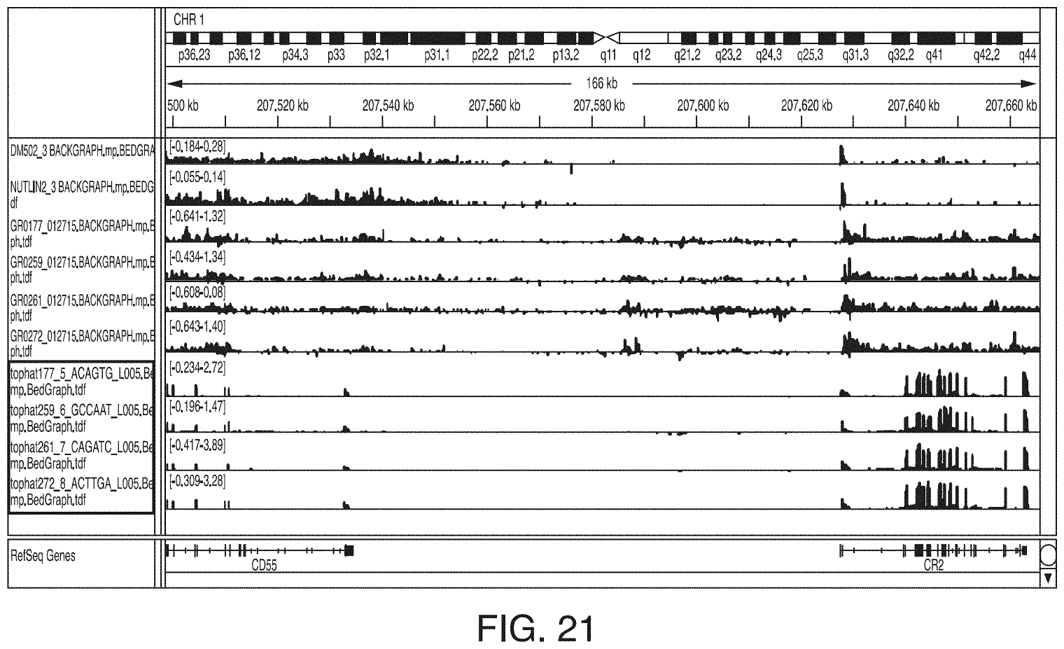

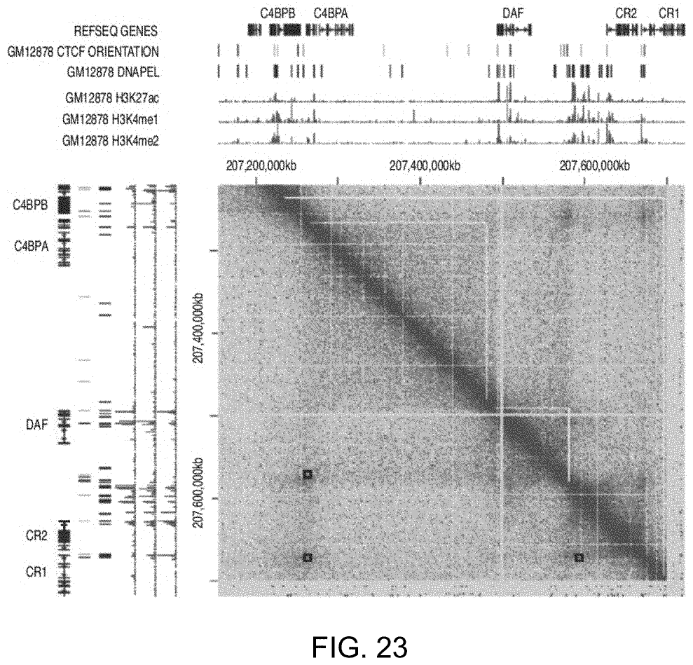

Alternatively, the SNP may alter binding of cell-type specific transcription factors, chromatin modifiers, and transcriptional activator complexes that increase the activity of the putative enhancer element. Finally, the 5′ region upstream of CR2 contains several additional putative B cell enhancer elements that are actively transcribed. FIG. 22 and FIG. 21, respectively. Since this locus interacts with the 5′ end of CR1 to form an anchor point for a chromatin loop, the SNP may modify the chromatin conformation within the loop and increase interaction of the intergenic enhancer region with the CR1 promoter. Rao et al., 2014. “A 3D map of the human genome at kilobase resolution reveals principles of chromatin looping” Cell 159: 1665-1680; and FIG. 23.

Both long noncoding RNA and enhancers can modify the regulation of neighboring and more distal genes due to colocalization of regulatory elements by chromatin looping, a process that is choreographed by CTCF. These data suggest several potential mechanisms by which the SNP could alter the transcription of CR1 independently of CR2. Since CR1 has immunomodulatory effects in B cells, it could contribute to the promotion of B cell tolerance, perhaps through the induction of a regulatory B cell phenotype. These data suggest that because lncRNA 9124 is positively associated with the protective haplotype, lncRNA 9124 may enhance CR1 transcription.

In one embodiment, LncRNA 9124 has the following nucleic acid sequence:

(SEQ ID NO: 1)
CCCACTGGCCCCTCCGGGAGCTGGGACCTCCAGAATTGGAGGCTGCGCCA
CAGAGGGGCGCTGTCTGGTCGGCCCGGTGTGGCTGGCGGCGTGCGGGTGC
TCGCGGTCTCCTGCCGGGCTCCCCGCCCCCAGGATTCTGCAGGTGCTCAT
CGCTCTCTGGCCGGCGGTCAGCGCTACCGCTCGCCACGGGAAACCTACCC
ACTGCATGAGGCACCAGCTAGCCACGTGAGGCTGTTTCTGTACACCCGAA
CCGCGCTCTTGCCCAGCAAAAGCGGCAGAACCTCGGCTCCCTTCCCTAAA
TAAGAGGATTTCTTGTGGCTCTCCTCCGCCTATCCTAAATGGCCGGATTA
GTTATTATTCTACCCCCATTGCTGTTGGTACCGTGATAAGGTACAGTTGT
TCAGGTACCTTCCGCCTCATTGGAGAAAAAAGTCTATTATGCATAACTAA
AGACAAAGTGGATGGAACCTGGGATAAACCTGCTCCTAAATGTGAATATT
TCAATAAATATTCTTCTTGCCCTGAGCCCATAGTAC

The CR2 lncRNA 9124 is believed to have at least two (2) exons, a first exon of 305 nucleotides taken from intron 1 of the CR2 gene, a second exon of 231 nucleotides taken from exon 2 of the CR2 gene, wherein the protective CR2 SNP is located 78 nucleotides downstream of exon 1 of the CR2 gene.

In other embodiments, the lncRNA may be encoded by, or within, nucleic acid sequences including, but not limited to,

lncRNA AJ1
(SEQ ID NO: 2)
GCTGTTTCTGTACACCCGAACCGCGCTCTTGCCCAGCAAAAGCGGCAGAA
CCTCGGCTCCCTTCCCTAAATAAGAGTAAGACCTGGTTCCCAAGCATGGG
TCTCAGATTTTCTGAACTCTAATGTGATATTGGTAGAGATCCGAAACATT
AACCATCCGTAGTTATTTTTGAGACATGGATTAAAACTATTGTTTTCTTC
TTGTTATTAACCTACTGTAATGGTGTGGACGTCCGCAAAGGTCTCATTCA
CCGTTTATTATATCTTTTTTTTCCTCCAGATTGGCTTGATTTTAAAGAAA
GATTATAATTAAGATTAGCGTCTCTTGGCTACAGGATGGGTCTAAGTTTC
TAATTATCTCCACGGTACCGTGCATTCCTAGTTTATAAGACGTTTACTGT
GTGCAGACCTCAATAACTGGGTGACTTTTGGCAAGTCATTTAGCTCCCTG
ATCTTCAGTTTCTTCATCTGTAAAATGAAACTAGTGATTAACTGGCCTCC
AAAAAAAAAAAA
lncRNA AJ2
(SEQ ID NO: 3)
GTTTCTGTACACCCGAACCGCGCTCTTGCCCAGCAAAAGCGGCAGAACCT
CGGCTCCCTTCCCTAAATAAGAGTAAGACCTGGTTCCCAAGCATGGGTCT
CAGATTTTCTGAACTCTAATGTGATATTGGTAGAGATCCTAAACATTAAC
CATCCGTAGTTATTTTTGAGACATGGATTAAAACTATTGTTTTCTTCTTG
TTATTAACCTACTGTAATGGTGTGGACGTCCGCAAAGGTCTCATTCACCG
TTTATTATATCTTTCTTTAAAATCAAGCCAATCTGGAGGAAAAAAA
lncRNA AJ3
(SEQ ID NO: 4)
GCTGTTTCTGTACACCCGAACCGCGCTCTTGCCCAGCAAAAGCGGCAGAA
CCTCGGCTCCCTTCCCTAAATAAGAGTAAGACCTGGTTCCCAAGCATGGG
TATCAGATTTTCTGAACTCTAATGTGATATTGGTAGAGATCCTAAACATT
AACCATCCGTAGTTATTTTTGAGACATGGATTAAAACTATTGTTTTCTTC
TTGTTATTAACCTACTGTAATGGTGTGGACGTCCGCAAAGGTTTCATTCA
CCGTTTATTATATCTTTCTTTAAAATCAAGCCAATGGGGAGGAAAAAAA
lncRNA AJ4
(SEQ ID NO: 5)
CTGTACACCCGAACCGCGCTCTTGCCCAGCAAAAGCGGCAGAACCTCGGC
TCCCTTCCCTAAATAAGAGTAAGACCTGGTTCCCAAGCATGGGTCTCAGA
TTTTCTGAACTCTAATGTGATATTGGTAGAGATCCTAAACATTAACCATC
CGTAGTTATTTTTGAGACATGGATTAAAACTATTGTTTTCTTCTTGTTAT
TAACCTACTGTAATGGTGTGGACGTCCGCAAAGGTCTCATTCACCGTTTA
TTATATCTTTTTTTCCTCCAGATTGGCTTGATTTTAAAGAAAGATTATAA
TTAAGATTAGCGTCTCTTGGCTACAGGATGGGTCTAAGTTTCTAATTATC
TCCACGGTACCGTGCATTCCTAGTTTATAAGACGTTTACTGTGTGCAGAC
CTCAATAACTGGGTGACTTTTGGCAAGTCATTTAGCTCCCTGATCTTCAG
TTTCTTCATCTGTAAAATGAAACTAGTGATTAACTGGCCTTCCACCTCAC
TGAGCTGCCCTGAGCACAGATGAAACTAAGCAAAGAAAGTTGGATGTTAG
TCGTTACTACTAAATATCAGTAATAAGCCTCCTGTACAGACTTGATTTTA
TGGAGCCAGCATTTAAGCTAACAAGCTGGGTGTCCAATTGTTTCTATGGA
CATCCAGCTTATTGAGGTGTCTGGAATACAGCACATATTAGTTTATTACA
ACTTTTTGCTTTCCTGACATTTTACTGTCTCCTGACCCTTAAACCTTTCT
CCAGACCATTATCTTCATCTCTTTTCTTTTTCTGCATATTAACTCCCTTT
GAACAAATGTATATTCACACTTTTTCTAAGAGCAGAGTACGTCCCTGACT
TCTCACCTATTTCTAAATTTCAAAGAGGATATTATCAACTGAAAATCAAA
TTCTTCACCAGTCCTGCCAATTCAGGGCCACAGCAGCAGCATCATGTAGG
TCACTTAACTGGAGCCCCATGAAATACCAGGTCTACCTATCAGCCATTCA
AGCTACTGTGCTTCTGTTGTTTGTCCATTTCTCATTTTTGTACCAGTATT
CCCATCCTAAGCATTATAATTACCTGTGTAACAACCTCATCTCTTTTAAG
AGATTTTGGTTCTTATGATTGGAAAGGTTAAAGAGTGACCTATAGGTCAC
TTTCCAATTATGAAAACAAAAAATTAAGAAATATATATATTTTCATTATT
TCACTCCATTGTTCAAAAATCTAAAAGGCCTCCAGTGGAAGAAAATCTGG
TGTCTTCAGCCTTTCTAGCCCTTTGGAATGGGATCTCACCGAAGCTCACA
CCCCCTGATTTCTCCAGCTGCCTGTCTTGTATGCGCTGCTCATGGTCTTC
CCCACCTGTCTCCCATGGCTCATGCTTTGCCCACTCTCCACTGCTAACTT
TTACTGCTACTGCATGCTCTCCAAAAAAAAAAA
lncRNA AJ5
(SEQ ID NO: 6)
GCTGTTTCTGTACACCCGAACCGCGCTCTTGCCCAGCAAAAGCGGCAGAA
CCTCGGCTCCCTTCCCTAAATAAGAGTAAGACCTGGTTCCCAAGCATGGG
TCTCAGATTTTCTGAACTCTAATGTGATATTGGTAGAGATCCTAAACATT
AACCATCCGTAGTTATTTTTGAGACATGGATTAAAACTATTGTTTTCTTC
TTGTTATTAACCTACTGTAATGGTGTGGACGTCCGCAAAGGTCTCATTCA
CCGTTTATTATATCTTTTTTTCCTCCAGATTGGCTTGATTTTAAAGAAAG
ATTATAATTAAGATTAGCGTCTCTTGGCTACAGGATGGGTCTAAGTTTCT
AATTATCTCCACGGTACCGTGCATTCCTAGTTTATAAGACGTTTACTGTG
TGCAGACCTCAATAACTGGGTGACTTTTGGCAAGTCATTTAGCTCCCTGA
TCTTCAGTTTCTTCATCTGTAAAATGAAACTAGTGATTAACTGGCCTTCC
ACCTCACTGAGCTGCCCTGAGCACAGATGAAACTAAGCAAAGAAAGTTGG
ATGTTAGTCGTTACTACTAAATATCAGTAATAAGCCTCCTGTACAGACTT
GATTTTATGGAGCCAGCATTTAAGCTAACAAGCTGGGTGTCCAATTGTTT
CTATGGACATCCAGCTTATTGAGGTGTCTGGAATACAGCACATATTAGTT
TATTACAACTTTTTGCTTTCCTGACATTTTACTGTCTCCTGACCCTTAAA
CCTTTCTCCAGACCATTATCTTCATCTCTTTTCTTTTTCTGCATATTAAC
TCCCTTTGAACAAATGTATATTCACACTTTTTCTAAGAGCAGAGTACGTC
CCTGACTTCTCACCTATTTCTAAATTTCAAAGAGGATATTATCAACTGAA
AATCAAATTCTTCACCAGTCCTGCCAATTCAGGGCCACAGCAGCAGCATC
ATGTAGGTCACTTAACTGGAGCCCCATGAAATACCAGGTCTACCTATCAG
CCATTCAAGCTACTGTGCTTCTGTTGTTTGTCCATTTCTCATTTTTGTAC
CAGTATTCCCATCCTAAGCATTATAATTACCTGTGTAACAACCTCATCTC
TTTTAAGAGATTTTGGTTCTTATGATTGGAAAGGTTAAAGAGTGACCTAT
AGGTCACTTTCCAATTATGAAAACAAAAAATTAAGAAATATATATATTTT
CATTATTTCACTCCATTGTTCAAAAATCTAAAAGGCCTCCAGTGGAAGAA
AATCTGGTGTCTTCAGCCTTTCTAGCCCTTTGGAATGGGATCTCACCGAA
GCTCACACCCCCTGATTTCTCCAGCTGCCTGTCTTGTATGCGCTGCTCAT
GGTCTTCCCCACCTGTCTCCCATGGCTCATGCTTTGCCCACTCTCCACTG
CTAACTTTTACTGCTACTGCATGCTCTCCAAAAAAAAAAA
lncRNA AJ6
(SEQ ID NO: 7)
GCTGTTTCTGTACACCCGAACCGCGCTCTTGCCCAGCAAAAGCGGCAGAA
CCTCGGCTCCCTTCCCTAAATAAGAGTAAGACCTGGTTCCCAAGCATGGG
TCTCAGATTTTCTGAACTCTAATGTGATATTGGTAGAGATCCTAAACATT
AACCATCCGTAGTTATTTTTGAGACATGGATTAAAACTATTGTTTTCTTC
TTGTTATTAACCTACTGTAATGGTGTGGACGTCCGCAAAGGTCTCATTCA
CCGTTTATTATATCTTTTTTTCCTCCAGATTGGCTTGATTTTAAAGAAAG
ATTATAATTAAGATTAGCGTCTCTTGGCTACAGGATGGGTCTAAGTTTCT
AATTATCTCCACGGTACCGTGCATTCCTAGTTTATAAGACGTTTACTGTG
TGCAGACCTCAATAACTGGGTGACTTTTGGCAAGTCATTTAGCTCCCTGA
TCTTACAGATGAAGAAACTGAAGAAACAAAAACTTATGGCACGGGAGTAA
ATTCAGCATTAAAATAAATGTAATTAAAAGAAAAAAAAAAA
lncRNA AJ7
(SEQ ID NO: 8)
GCTGTTTCTGTACACCCGAACCGCGCTCTTGCCCAGCAAAAGCGGCAGAA
CCTCGGCTCCCTTCCCTAAATAAGAGTAAGACCTGGTTCCCAAGCATGGG
TCTCAGATTTTCTGAACTCTAATGTGATATTGGTAGAGATCCTAAACATT
AACCATCCGTAGTTATTTTTGAGACATGGATTAAAACTATTGTTTTCTTC
TTGTTATTAACCTACTGTAATGGTGTGGACGTCCGCAAAGGTCTCATTCA
CCGTTTATTATATCTTTTTTTCCTCCAGATTGGCTTGATTTTAAAGAAAG
ATTATAATTAAGATTAGCGTCTCTTGGTTACAGGATGGGTCTAAGTTTCT
AATTATCTCCACGGTACCGTGCATTCCTAGTTTATAAGACGTTTACTGTG
TGCAGACCTCAATAACTGGGTGACTTTTGGCAAGTCATTTAGCTCCCTGA
TCTTCAGTTTCTTCATCTGTAAAATGAAACTAGTGATTAACTGGCCAAAA
AAAAAAA

The above representative lncRNAs were identified by 3′RACE using the Ambion FirstChoice RLM-RACE Kit. The outer and inner gene-specific primers were located within the first exon of lncRNA transcript 2 (intron 1 sequence of CR2 gene). These sequences represent raw data derived from preliminary sequencing of 16 clones and may contain random sequencing errors as readily recognized by one of ordinary skill in the art.

C. SLE Phenotypic Receptor Expression

The B cell complement receptors, complement receptor 1 (CR1/CD35) and complement receptor 2 (CR2/CD21), may play roles in the maintenance of tolerance to self-antigens that is causally associated with systemic lupus erythematosus (SLE). In mouse models of lupus, these receptors decrease prior to the onset of clinical disease; similarly, patients with SLE demonstrate a 50% decrease in CR1 and CR2 protein levels. Takahashi et al., 1997 “Mouse complement receptors type 1 (CR1; CD35) and type 2 (CR2; CD21): expression on normal B cell subpopulations and decreased levels during development of autoimmunity in MRL/lpr mice” J. Immunol. 159: 1557-1569; Wilson et al., 1986 “Decreased expression of the C3b/C4b receptor (CR1) and the C3d receptor (CR2) on B lymphocytes and of CR1 on neutrophils of patients with systemic lupus erythematosus” Arth. Rheum. 29: 739-747; Levy et al., 1992 “T lymphocyte expression of complement receptor 2 (CR2/CD21): a role in adhesive cell-cell interactions and dysregulation in a patient with systemic lupus erythematosus (SLE)” Clin. Exp. Immunol. 90: 235-244; and Marquart et al., 1995 “Complement receptor expression and activation of the complement cascade on B lymphocytes from patients with systemic lupus erythematosus (SLE)” Clin. Exp. Immunol. 101: 60-65.

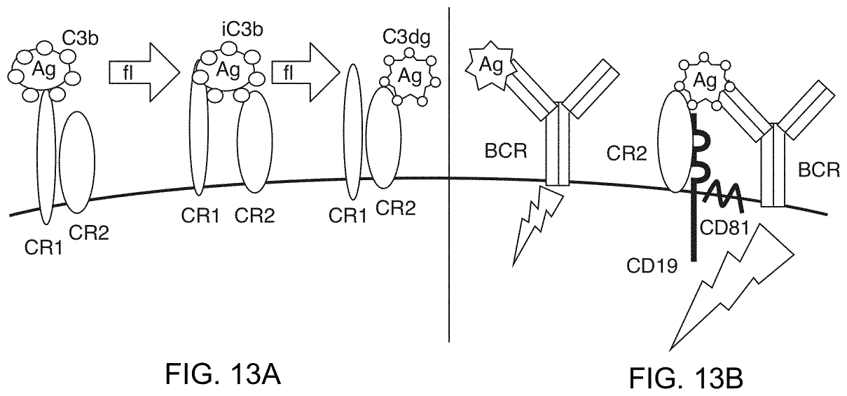

Functional relevance to this change may be suggested by the finding that inactivation of a Cr2 allele, which encodes both CR1 and CR2 in mice by alternative splicing, worsens lupus manifestations. Prodeus et al., 1998 “A critical role for complement in the maintenance of self-tolerance” Immunity 9:721-731; and Wu et al., 2002 “A role for the CR2 gene in modifying autoantibody production in systemic lupus erythematosus” J. Immunol. 169:1587-1592. CR1/CR2 also play a role in normal immune responses. For example, CR1 binds C3b and C4b activation fragments of C3 and C4 and serves as a cofactor for factor I-mediated cleavage of C3b to the terminal C3 degradation fragments, iC3b and C3dg, which are CR2 ligands. Ahearn et al., 1989 “Structure and function of the complement receptors, CR1 (CD35) and CR2 (CD21)” Adv Immunol 46: 183-219; and FIGS. 13A-13B.

CR1 and CR2 receptors form a non-covalent complex on the B cell membrane that is believed to facilitate B cell activation by efficiently capturing C3b-coated antigens and loading them on to CR2 after cleavage of C3b. CR2 cooperates with the B cell receptor (BCR) to activate B cells, processes and presents complement-coated antigens to T cells, and shapes the natural antibody repertoire (11). Independent ligation of CR1 is believed to mediate inhibitory signals in human B cells (12), although the mechanism for this has not been well characterized. Holers V M. 2005. “Complement receptors and the shaping of the natural antibody repertoire. Springer Semin Immun 26: 405-423; and Józsi et al., 2002. “Complement receptor type 1 (CD35) mediates inhibitory signals in human B lymphocytes. J Immunol 2002: 2782-2788

A high expression of lncRNA 9124 was observed in adrenal, lymph node, and white blood cells. Consequently, targeting the CR2 lncRNA in lupus patients to increase B cell CR1 levels may induce specific B cell tolerance and sustained remission from disease. Further, B cells show a higher CR1 surface expression for the AA/AG minor haplotype than for the major GG haplotype.

CR1 and CR2 receptors are both believed reduced by ˜50% on B cells in SLE patient. For example, CR2 is displayed on B cells and follicular dendritic cells (FDCs) and are involved in B cell activation. On the other hand, CR1 is displayed on B cells, FDCs, erythrocytes, monocytes, neutrophils, and/or podocytes and are involved in B cell inhibition. Tuveson, et al, J Exp Med 173:1083 (1991).

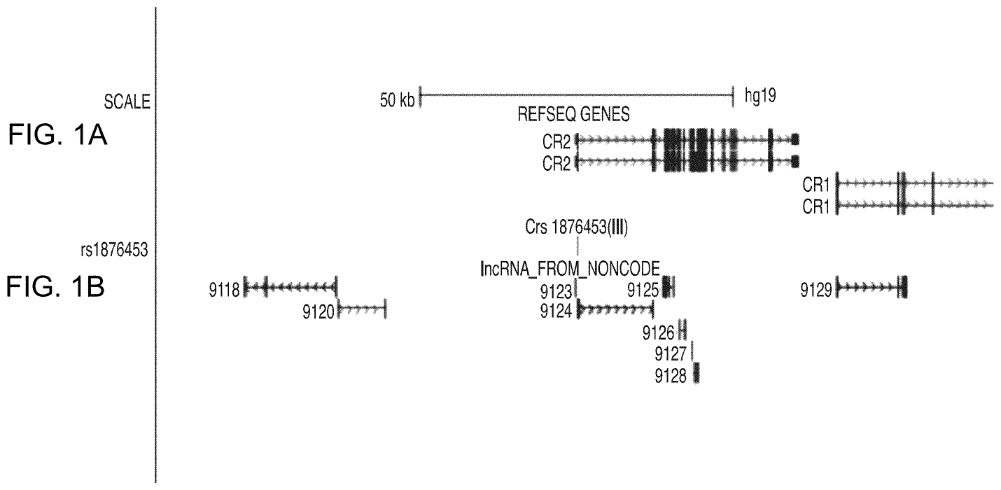

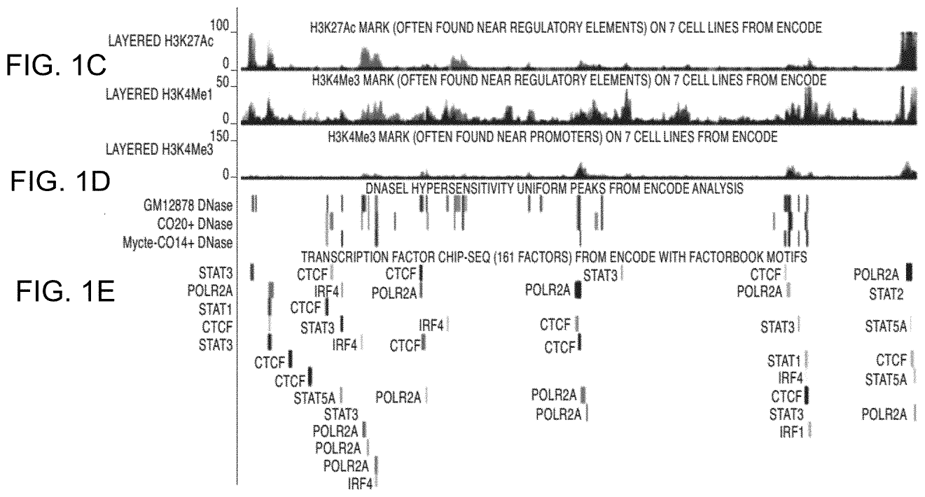

CR1:CR2 ratio on B cells was observed to be higher in patients with the minor allele A whereas differences in CR1 expression were not detected on other types of peripheral blood cells. Thus, the rs1876453 chromosome location may have long-range effects on the regulation of expression of the CR1 gene (which is proximately upstream to the CR2 gene), that are either B cell-specific or dependent on co-expression of CR2. The rs1876453 chromosome location is 97 nucleotides downstream of the CR2 Intron 1. See FIGS. 1A-E. The CR2 Intron 1 is 12 kb and contains a conserved silencing domain that controls CR2 expression in a cell type-specific and developmentally regulated manner.

Expression of CR2 in B cells generally occurs between a late immature and transitional developmental stage and CR1 is expressed earlier. These changes convert the B cell from a CR2low designation to a CR2high designation, as the cells mature, some into regulatory B cells and this state is believed as a checkpoint for induction of tolerance of self-reactive B cells by a deletion pathway.

A 3255 bp RNA sequence having a portion that is similar to SEQ ID NO:1 has been reported that was integrated into an expression vector for transformation of a host human cell. The transformed cell culture stimulated B cells to grow and/or differentiate. The polynucleotide was suggested to either stimulate, enhance or inhibit B cells and may be useful in therapeutic applications, such as for systemic lupus erythematosus. Keting, et al., WO 2004/020595. “Novel Human Polypeptides Encoded By Polynucleotides.

Regulatory B cells are generated under inflammatory conditions and are not detected in normal states. They produce inhibitory cytokines, including IL-10, TGFβ, and IL-35, and their immunoregulation is antigen specific, making them prime targets for the treatment of autoimmune disease. Mizoguchi et al., 2002. “Chronic intestinal inflammatory condition generates IL-10-producing regulatory B cell subset characterized by CD1d upregulation” Immunity 16: 219-230; Yanaba et al., 2008. “A regulatory B cell subset with a unique CD1dhiCD5+ phenotype controls T cell-dependent inflammatory responses” Immunity 28:639-650; Lee et al., 2014. “TGF-beta-producing regulatory B cells induce regulatory T cells and promote transplantation tolerance” Eur J Immunol 44: 1728-1736; Wang et al., 2014. “Interleukin-35 induces regulatory B cells that suppress autoimmune disease. Nat Med 20: 633-641; Yanaba et al., 2009. “The development and function of regulatory B cells expressing IL-10 (B10 cells) requires antigen receptor diversity and TLR signals’ J Immunol 182:7459-7472; and Evans et al, 2007. “Novel suppressive function of transitional 2 B cells in experimental arthritis. J Immunol 178:7868-7878.

The B cell complement receptors, CR1 and CR2, are believed to play roles in the maintenance of tolerance to self-antigens that is causally associated with SLE. In mouse models of lupus, these receptors decrease prior to the onset of clinical disease; similarly patients with SLE demonstrate a 50% decrease in CR1 and CR2 protein levels. Takahashi et al., 1997. “Mouse complement receptors type 1 (CR1; CD35) and type 2 (CR2; CD21): expression on normal B cell subpopulations and decreased levels during development of autoimmunity in MRL/lpr mice” J. Immunol. 159: 1557-1569; Wilson et al., 1986. “Decreased expression of the C3b/C4b receptor (CR1) and the C3d receptor (CR2) on B lymphocytes and of CR1 on neutrophils of patients with systemic lupus erythematosus. Arth. Rheum. 29: 739-747; Levy et al. 1992. “T lymphocyte expression of complement receptor 2 (CR2/CD21): a role in adhesive cell-cell interactions and dysregulation in a patient with systemic lupus erythematosus (SLE)” Clin. Exp. Immunol. 90: 235-244; and Marquart et al., 1995. “Complement receptor expression and activation of the complement cascade on B lymphocytes from patients with systemic lupus erythematosus (SLE)” Clin. Exp. Immunol. 101: 60-65. Functional relevance to this change is suggested by the finding that inactivation of the CR2 allele, which encodes both CR1 and CR2 in mice by alternative splicing, worsens lupus manifestations. Prodeus et al., 1998. “A critical role for complement in the maintenance of self tolerance” Immunity 9: 721-731; and Wu et al., 2002. “A role for the Cr2 gene in modifying autoantibody production in systemic lupus erythematosus” J. Immunol. 169: 1587-1592.

The data presented herein suggest that CR1 and CR2 contribute to the suppression of autoimmune disease through the induction of regulatory B cells specific for autoantigens encountered in the context of inflammation. A potential mechanism has been identified by which increased CR1 transcription associated with an rs1876453 SNP could be protective for SLE. Although it is not necessary to understand the mechanism of an invention, it is believed that CR1 modulates the generation of regulatory B cells and suppresses autoimmune disease, so that therapeutic approaches targeting CR1 or the pathways it triggers can be developed for the treatment of lupus and other autoimmune diseases.

D. Type I Diabetes

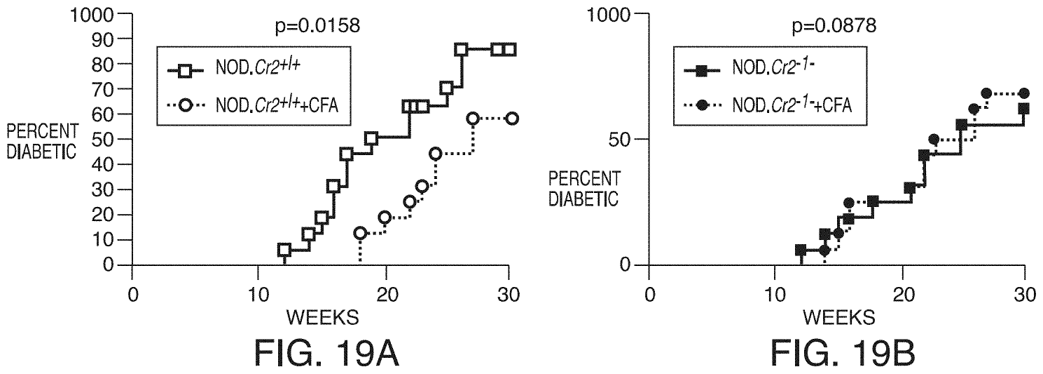

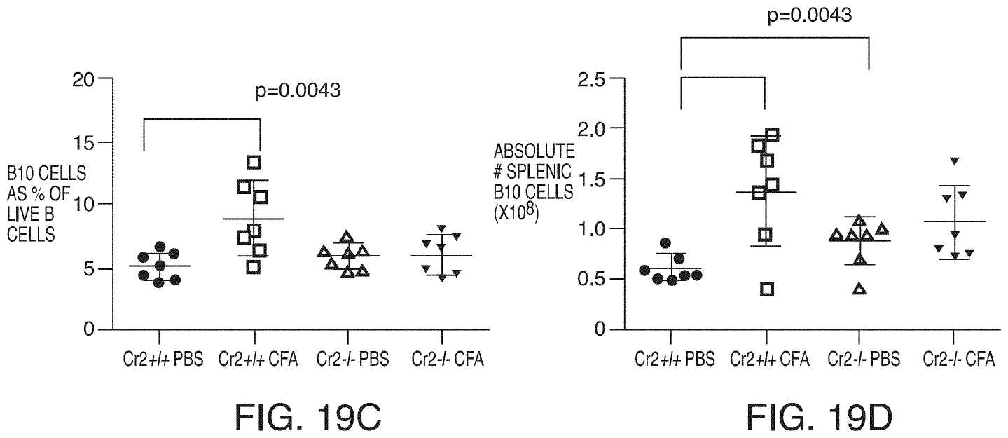

In one embodiment, the present invention contemplates that CR1/CR2 gene transcription regulation may play a role in immune tolerance. This was evaluated using the adjuvant-induced model of type I diabetes prevention. Female Non-Obese Diabetic (NOD) mice develop inflammation in the pancreatic islets at 3-5 weeks of age that leads to β cell destruction and overt diabetes by 4-6 months of age. A single injection of Complete Freund's Adjuvant (CFA) given between the ages of 4 and 10 weeks delays the development of hyperglycemia by inducing suppressor cells in the spleen that are maximally generated 8-10 days after injection. Sadelain et al., 1990.” Prevention of type I diabetes in NOD mice by adjuvant immunotherapy” Diabetes 39:583-589. It was found that while unmanipulated mice sufficient or deficient for CR1/CR2 had similar patterns of diabetes development (p=0.1898), only mice sufficient for CR1/CR2 had delayed onset and reduced incidence of diabetes in response to CFA injection. FIG. 19A versus FIG. 19B. This difference in response to CFA was associated with impaired expansion of regulatory B cells in the spleen termed B10 cells, which were more than 2-fold increased in CR1/CR2-sufficient mice but not expanded in deficient mice. FIG. 19C and FIG. 19D. CR1/CR2-deficient mice that received PBS alone did have ˜20% more B10 cells than CR1/CR2-sufficient, but this did not appear to have an effect on diabetes development.

Enhancers are DNA sequences that recruit transcription factors to regulate the transcription of target genes in a cell type-specific manner. Enhancers act as platforms for the binding of integrated cell-type specific transcription factors, and they can be located many thousands of kilobases away from their target genes. Active enhancers also recruit RNA polymerase II (Pol II), leading to the production of enhancer-originating, short bidirectional RNAs of ˜500 nucleotides, termed eRNAs. In addition to serving as binding platforms for cell-type specific transcription factors, enhancers are occupied by DNA-binding effectors of cell signaling pathways and they give these signals cell-type specificity. Input from multiple enhancers can be integrated at a single promoter and likewise, information from a single enhancer can be conveyed to multiple promoters for coordinated gene regulation. Buecker et al., 2012. “Enhancers as information integration hubs in development: lessons from genomics” Trends Genet 28: 276-284.

Enhancer transcription correlates with enhancer activity and is the earliest event in successive waves of coordinated transcription during cellular activation. Wang et al., 2011. “Reprogramming transcription by distinct classes of enhancers functionally defined by eRNA” Nature 474: 390-394; and Arner et al., 2015. “Gene regulation. Transcribed enhancers lead waves of coordinated transcription in transitioning mammalian cells” Science 347:1010-104. An increasing numbers of reports suggest that this transcriptional activity play an active role for enhancer function.

Active enhancers can be identified based on DNase hypersensitivity, enhancer-specific histone modifications, and transcription factor binding, but one of the best ways to identify transcriptionally active enhancers is by global run-on sequencing (GRO-Seq). This approach involves the extension of nascent RNA that are associated with transcriptionally engaged polymerases under conditions where new initiation is prohibited. Core et al., 2008. “Nascent RNA sequencing reveals widespread pausing and divergent initiation at human promoters” Science 322: 1845-1848. A ribonucleotide analog (e.g., 5-bromouridine 5′triphosphate (BrU)) is added during the run-on step to tag the nascent RNA and enable it to be immunopurified. Nascent RNA is then chemically hydrolyzed into short fragments that are subjected to high throughput sequencing. The origin and orientation of the RNAs and the associated transcriptionally engaged polymerases can then be documented genome-wide by mapping the reads to the reference human genome. GRO-Seq has high sensitivity and low background, and it can identify the position of transcripts induced by activating stimuli. Furthermore, it is one of the best ways to study the function of eRNAs, which due to their instability and low frequency, are not detected by RNA-Seq. FIG. 21. Since both eRNAs and their target coding transcripts can be induced by activating stimuli, GRO-Seq provides a way to link extracellular signals to enhancer function and gene regulation.

RNA-Seq involves isolating a population of RNA, converting it to a library of cDNA fragments with attached adaptors, and sequencing the cDNA library to obtain short reads of 30-400 nucleotides in length. These sequences are mapped to a reference genome and the expression level for various genes or transcripts can be determined by counting the number of reads that align to its exons. In contrast to the microarrays that have been historically used for gene expression studies, RNA-Seq can capture subtle gene expression changes that may be masked by noise levels caused by cross-hybridization to probes with similar sequences and it is not biased by previous assumptions about the transcriptome. Furthermore, it can identify alternative splicing events, allele-specific expression, rare and novel transcripts, and non-coding RNA. RNA-Seq has been shown to be an effective tool for the evaluation of the expression and structure of genes in human B cells. Toung et al., 2011. “RNA-sequence analysis of human B-cells” Genome Res 21: 991-998.

Global run-on sequencing (GRO-Seq) complements the gene expression data provided by RNA-Seq. It involves the extension of nascent RNA that are associated with transcriptionally engaged polymerases under conditions where new initiation is prohibited. Core et al., 2008. “Nascent RNA sequencing reveals widespread pausing and divergent initiation at human promoters” Science 322:1845-1848. A ribonucleotide analog (for example, 5-bromouridine 5′triphosphate (BrU)) is added during the run-on step to tag the nascent RNA and enable it to be immunopurified. Nascent RNA is then chemically hydrolyzed into short ˜150 base fragments that are subjected to high throughput sequencing. The origin and orientation of the RNAs and the associated transcriptionally engaged polymerases can then be documented genome-wide by mapping the reads to the reference human genome. GRO-Seq has high sensitivity and low background, and it can identify the position of transcripts induced by activating stimuli. Furthermore, it is one of the best ways to study the function of eRNAs, which due to their instabilitABy and low frequency, are not detected by RNA-Seq. Since both eRNAs and their target coding transcripts can be induced by activating stimuli, GRO-Seq provides a way to link enhancer function to gene regulation.

To determine the optimal time after stimulation for preparation of nuclei for GRO-Seq, conditions used will be the same as for induction of regulatory B cells. Three NOD mice will be killed and splenocytes sorted for subsets enriched for regulatory B cells 1, 3, 12, and 24 hours after treatment. Three control mice will be given PBS before being killed. For human samples, PBMCs will be obtained from 3 subjects with optimized activation conditions or media for 20, 40, or 60 minutes before sorting for B cell subsets in which regulatory B cells are enriched. For both mouse and human cells, nuclei are prepared and perform nuclear run-on reactions followed by PCR to amplify transcripts associated with the IL10 gene; the time point at which we obtain maximal numbers of IL10-associated transcripts will be used for GRO-Seq.

To prepare the nuclei 25 CR1/CR2-sufficient and 25 CR1/CR2-deficient NOD mice will be treated with stimulus or PBS then killed. B cell subsets enriched with regulatory B cells will be sorted and nuclei prepared. An additional 50 healthy African-American (AA) non-pregnant women between the ages of 18 and 45 will be enrolled, 25 of whom are homozygous or heterozygous for the minor protective allele for rs1876453 and 25 of whom are homozygous for the major allele, requiring an additional 158 subjects to be recruited. Only premenopausal women are included, because GRO-Seq data can vary for sex-specific reasons and because the incidence of lupus is highest in this demographic group. Enrolled subjects will be invited for a study visit at which time up to 300 milliliters of peripheral blood will be collected by standard phlebotomy. PBMCs will be incubated under optimized activation conditions or media alone, after which B cell subsets enriched for regulatory B cells will be sorted and nuclei prepared.

GRO-Seq nuclear run-on assays will be performed to extend nascent transcripts that are associated with transcriptionally engaged polymerases under conditions where new initiation is prohibited. Nascent RNAs will be tagged with BrUTP, chemically hydrolyzed into ˜150 bp fragments, and immunopurified using anti-BrU beads. cDNA libraries will be prepared and subjected to single-end sequencing on an Illumina HiSeq platform. Each lane will contain 4 libraries for a read depth of 100-120 million single-end reads of 50 nucleotides. Read mapping, transcriptome reconstruction, and expression quantification will be performed. Azofeifa et al., 2014. “FStitch: A fast and simple algorithm for detecting nascent RNA transcripts” 5th ACM Conference on Bioinformatics, Computational Biology and Health Informatics.

A paired analysis in R will be performed for the human studies that corrects for any covariates such as age or hormonal status (for women) if we were not able to match for these. Multiple comparisons will be adjusted for by using a false discovery rate. After identifying the top differentially expressed genes, Ingenuity Pathway Analysis is applied to identify pathways associated with these genes that are modified as a result of the protective SNP.

Validation experiments will be performed in the independent cohort of using nuclear run-on, quantitative RT-PCR, flow cytometry, and immunofluorescence microscopy. Up to 50 of the top differentially expressed transcripts will be validated.

A power analysis using RNASeqPower for a two-group comparison indicates that there is >97% power to detect effect sizes of at least 2 for expression between the two groups with a sample size of n=25 per group. Hart et al., 2013. “Calculating sample size estimates for RNA sequencing data” J Comput Biol 20: 970-978. Previous GRO-Seq experiments suggest that the typical enhancer fold-change was 3-fold. Allen et al., 2014. “Global analysis of p53-regulated transcription identifies its direct targets and unexpected regulatory mechanisms” Elife 3: e02200 Average depth per gene is assumed to be 10 and the coefficient of variation for biological replicates to be 0.20. Eight comparisons will be made (1 perturbation, 2 mouse or human groups, and up to 4 cell populations) and 50,000 features (genes and enhancer RNAs) are expected to be identified. The alpha level is set at 6.3e-06, which corresponds to a conservative Bonferroni correction for multiple comparisons that would result in at most one false positive statement for the 50,000*8 tests for each gene.

CR1/CR2 are believed to play a role in the induction of regulatory B cells under inflammatory conditions that suppress the onset of autoimmune diabetes. CR1 is also increased in human B cells in association with a CR2 SNP that is protective for lupus. An overexpression of CR1 in two mouse models of autoimmune disease, might delay the onset and alter the progression of disease. This reflects a strategy to prevent autoimmune disease in at-risk individuals who have evidence of autoimmunity but no symptoms.

CR1 and CR2 are derived from alternatively spliced transcripts of a single gene in mice. Therefore, to evaluate the relative contributions of each protein to the induction of regulatory B cells that delay disease onset and reduce severity, bone marrow chimeras are generated that overexpress CR1, CR2 or both proteins on an autoimmune-prone background. The recipients will be Cr2+/+ NOD or SNF1 mice and the donor hematopoietic stem cells (HSC) will be derived from Cr2+/+ or Cr2−/− syngeneic mice. A new lentiviral construct is generated in which the cDNA for CR1 or CR1 have been cloned under the control of regulatory elements that ensure their stage- and cell-type specific expression. After injection of transduced HSC, engrafted mice will be injected with CFA/immune agonist or PBS and followed for disease development.

V. Autoimmune Disease Treatment By lncRNA Targeting

Systemic lupus erythematosus is a multisystemic autoimmune disease characterized by the production of autoantibodies to nuclear antigens. Loss of tolerance to nuclear antigens precedes the development of clinically apparent disease in individuals with systemic lupus erythematosus (SLE), but only ˜5% of healthy individuals with a positive anti-nuclear antibody (ANA) go on to develop disease. The genetic and environmental factors that influence progression from lupus autoimmunity to disease are not known; identifying them may lead to a new treatment for lupus. The B cell complement receptors, complement receptor 1 (CR1/CD35) and complement receptor 2 (CR2/CD21), play essential roles in the maintenance of tolerance to self-antigens that is impaired in SLE.

The data presented herein identifies a variant in intron 1 of complement receptor 2 (CR2/CD21) that is associated with decreased risk of lupus (r51876453; Pₘₑₜₐ=4.2×10⁴, OR=0.85). Its effect was strongest in subjects with anti-dsDNA antibodies (case-control Pₘₑₜₐ=7.6×10⁻⁷, OR=0.71; case-only Pₘₑₜₐ=1.9×10⁴, OR=0.75), suggesting a preferential association with this endophenotype. rs1876453, located 97 nucleotides from the 5′ end of CR2 intron 1, alters the binding of multiple protein complexes, including one containing CTCF, and is associated with increased B cell-specific expression of the adjacent upstream gene, complement receptor 1 (CR1/CD35).

The presence of annotated lncRNAs in the CR2-CR1 genomic region in various cell types was investigated. One annotated lncRNA was located 78 nucleotides downstream of the rs1876453 SNP in CR2 intron 1 and was readily detected in B cells. Allele-specific expression of this lncRNA was detected by quantitative RT-PCR and found that it was ˜3-fold increased in individuals with the minor protective allele at rs1876453 (p=0.0025 normalized to U6 snRNA and p=0.0054 normalized to beta-actin). Although the specific functions of lncRNAs are varied and incompletely understood, numerous associations between disease states and lncRNA alterations have now been reported. The data presented herein suggest that the generation of pathogenic autoantibodies associated with early, active, and severe lupus is modified by expression of a CR2 lncRNA that appears to have long-range effects. Examination of its mechanism and effects may therefore reveal a therapeutic target for the treatment of lupus.

The present data shows an lncRNA that has been mapped to the first intron and second exon of human CR2 that is overexpressed in individuals who are homozygous or heterozygous for the minor allele at rs1876453. Increased expression of the lncRNA may be responsible for decreased binding of CTCF at this genomic locus as well as long-range effects on CR1 transcription. Although it is not necessary to understand the mechanism of an invention, it is believed that the lncRNA may mediate an association of an rs1876453 minor allele A with a reduced risk of lupus and specifically with the antibodies to dsDNA that are associated with preclinical, active, and severe disease.

The data presented herein suggest that:

i) intronic CR2 SNP is associated with decreased risk of lupus and preferentially decreases risk of anti-dsDNA antibodies;

ii) a protective SNP (r51876453) is associated with: a) an increased expression of CR1; b) an altered formation of multiple protein-DNA complexes, including one containing CTCF; and c) an increased level of a nearby sense long noncoding RNA; and

iii) targeting a CR2 lncRNA in lupus to increase B cell CR1 levels may induce specific B cell tolerance and provide a sustained SLE remission.

IV. Pharmaceutical Formulations

The present invention further provides pharmaceutical compositions (e.g., comprising the compounds described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the compound is administered in maintenance doses, ranging from 0.01 μg to 100 g per kg of body weight, once or more daily, to once every 20 years.

Furthermore, the compositions disclosed herein (i.e., for example, RNA sequences and/or expression constructed encoded for such sequences) may be administered in as a stabilized RNA complex. Stabilized RNA complexes have been reported in improve circulating half-lives by avoiding exposure to nucleases and other clearance mechanisms. Hoerr et al., U.S. Pat. No. 9,463,228 (herein incorporated by reference).

In contrast to DNA, the use of RNA as a gene therapeutic or vaccine is to be classified as substantially safer. In particular, RNA does not involve the risk of being integrated into the genome of the transfected cell in a stable manner. Furthermore, no viral sequences, such as promoters, are necessary for effective transcription. Moreover, RNA is degraded considerably more easily in vivo. Apparently because of the relatively short half-life of RNA in the blood circulation compared with DNA, no anti-RNA antibodies have been detected to date. RNA can therefore be regarded as the molecule of choice for molecular medicine therapy methods. Nevertheless, medical methods based on RNA expression systems still require a solution to some fundamental problems before they are used more widely. One of the problems of using RNA is reliable cell- or tissue-specific efficient transfer of the nucleic acid. Since RNA usually proves to be very unstable in solution, it has not hitherto been possible, or has been possible only in a very inefficient manner, to use RNA as a therapeutic or vaccine by the conventional methods which are used with DNA.

RNA-degrading enzymes, so-called RNAases (ribonucleases), are responsible for the instability. Even the smallest impurities of ribonucleases are sufficient to degrade RNA in solution completely. The natural degradation of mRNA in the cytoplasm of cells is very finely regulated. Several mechanisms are known in this respect. Thus, the terminal structure is of decisive importance for a functional mRNA. At the 5′-end is the so-called “cap structure” (a modified guanosine nucleotide), and at the 3′-end a sequence of up to 200 adenosine nucleotides (the so-called poly-A tail). The RNA is recognized as mRNA and the degradation is regulated via these structures. Moreover, there are further processes which stabilize or destabilize RNA. Many of these processes are still unknown, but an interaction between the RNA and proteins often appears to be decisive for this. For example, an “mRNA surveillance system” has recently been described (Hellerin and Parker, Annu. Rev. Genet. 1999, 33: 229 to 260), in which incomplete or nonsense mRNA is recognized by certain feedback protein interactions in the cytosol and is rendered accessible to degradation, the majority of these processes being performed by exonucleases.

Some measures for increasing the stability of RNA and thereby rendering possible its use as a gene therapeutic or RNA vaccine have been proposed in the prior art.

To solve the abovementioned problems of the instability of RNA ex vivo, EP-A-1083232 proposes a process for introduction of RNA, in particular mRNA, into cells and organisms, in which the RNA is in the form of a complex with a cationic peptide or protein.

WO 99/14346 describes further processes for stabilizing mRNA. In particular, modifications of the mRNA which stabilize the mRNA species against the degradation by RNases are proposed. Such modifications concern on the one hand stabilization by sequence modifications, in particular reduction of the C and/or U content by base elimination or base substitution. On the other hand, chemical modifications, in particular the use of nucleotide analogues, and 5′- and 3′-blocking groups, an increased length of the poly-A tail and complexing of the mRNA with stabilizing agents and combinations of the measures mentioned, are proposed.

The U.S. Pat. Nos. 5,580,859 and 6,214,804 disclose, inter alia, mRNA vaccines and therapeutics in the context of “transient gene therapy” (TGT). Various measures for increasing the translation efficiency and the mRNA stability based above all on untranslated sequence regions are described.

The mRNA has, in the 5′- and/or 3′-untranslated regions, stabilizing sequences which are capable of increasing the half-life of the mRNA in the cytosol. These stabilizing sequences can have a 100% sequence homology to naturally occurring sequences which occur in viruses, bacteria and eukaryotes, but can also be partly or completely of synthetic nature. Examples of stabilizing sequences which can be used in the present invention and which may be mentioned are the untranslated sequences (UTR) of the .beta.-globin gene, e.g. from Homo sapiens or Xenopus laevis. Another example of a stabilizing sequence has the general formula (C/U)CCANₓCCC(U/A)PyₓUC(C/U)CC, which is contained in the 3′-UTR of the very stable mRNA which codes for .alpha.-globin, .alpha.-(I)-collagen, 15-lipoxygenase or for tyrosine hydroxylase (cf. Holcik et al., Proc. Natl. Acad. Sci. USA 1997, 94: 2410 to 2414). Such stabilizing sequences can of course be used individually or in combination with one another and also in combination with other stabilizing sequences known to a skilled person in the art.

### EXPERIMENTAL

### Example I

### RT-PCR Confirmation of lncRNA Expression

cDNA was generated by reverse transcription from RNA purified from cell lines and primary cells. PCR was performed using 5′ and 3′ primers that targeted spliced exons from known lncRNA sequences in the intergenic region 5′ of CR2 (9118, 9120), in the CR2 gene (9124, 9125, 9126, 9127, 9128), and in CR1 intron 1 (9129). Template RNA was derived from Raji (B cell lymphoma) and HK (follicular dendritic cell) cell lines as well as from human tonsil and spleen (homogenized tissue), peripheral blood mononuclear cells (PBMC), and purified primary B cells. The data is shown below:

| IncRNA | Location | Detection |
|---|---|---|
| 9118 | Upstream active region | B cell |
| 9120 | Upstream active region | None detected (only Raji tested) |
| 9123 | CR2 (near SNP) | Not Done (1 exon) |
| 9124 | CR2 (near SNP) | Raji, Tonsil, PBMC, B cell |
| 9125 | CR2 | Raji, Spleen, Tonsil, PBMC |
| | | (no B cell data) |
| 9126 | CR2 | Raji, Spleen, Tonsil, PBMC |
| | | (no B cell data) |
| 9127 | CR2 | Not Done (1 exon) |
| 9128 | CR2 (exon 11) | Raji, HK, Spleen, Tonsil, PBMC |
| | | (no B cell data) |
| 9129 | CR1 | B cell |

### Example II

### Allele-Specific Differences in Expression of lncRNA 9124

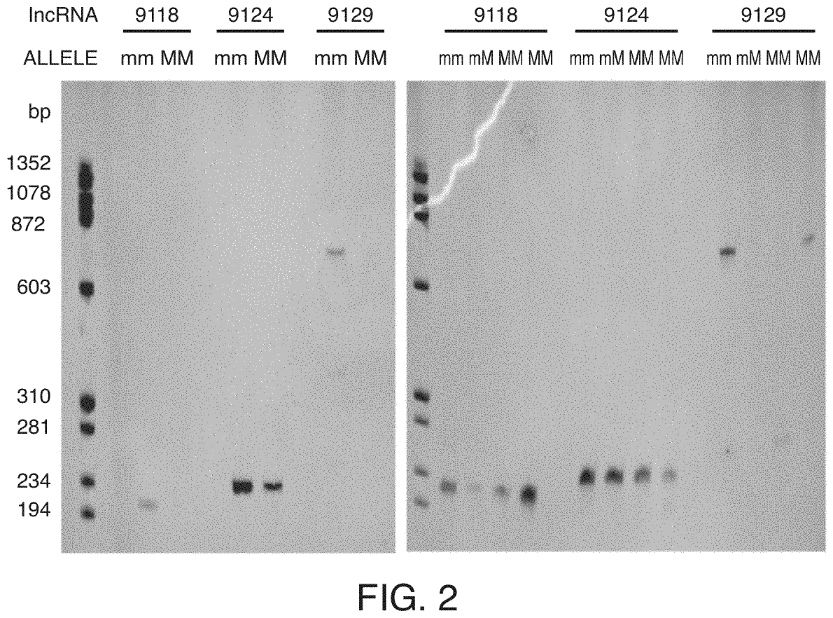

cDNA was generated by reverse transcription from RNA purified from subjects with the minor (homozygous: mm; heterozygous: mM) or major allele (MM) at rs1876453. PCR was performed using 5′ and 3′ primers that targeted spliced exons from known lncRNA sequences in the intergenic region 5′ of CR2 (9118), in CR2 intron 1 (9124), and in CR1 intron 1 (9129). Products were separated on a 7.5% acrylamide gel; the size of each product was as predicted based on splicing patterns of published lncRNAs and results represent two independent experiments See, FIG. 2.

### Example III

### Sequencing Of Amplified cDNA From lncRNA 9124

Nucleic acid bands were extracted from an SDS gel following electrophoresis and sequenced in a conventional autoanalyzer. cDNA derived from subjects homozygous for either the major or minor allele were identical and matched the published sequence for lncRNA 9124 as shown below:

| | |
|---|---|
| lnc19124 | TGAGGCTGTTTCTGTACACCCGAACCGCGCTCTTGCCCAGCAAAAGCGGCAGAACCTCGG |
| minor | [symbol] |
| major | [symbol] |
| lnc9124 | CTCCCTTCCCTAAATAAGAGGATTTCTTGTGGCTCTCCTCCGCCTATCCTAAATGGCCGG |
| minor | [symbol] |
| major | [symbol] |
| | [symbol] |
| | Exon junction |
| lnc9124 | ATTAGTTATTATTCTACCCCCATTGCTGTTGGTACCGTGATAAGGTACAGTTGTTCAGGT |
| minor | [symbol] |
| major | [symbol] |
| | ------------------------------------------------------------ |
| lnc9124 | ACCTTCCGCCTCAT |
| minor | [symbol] |
| major | [symbol] |
| | [symbol] |

[symbol]

indicates data missing or illegible when filed

### Example IV

### lncRNA Haplotype Analysis

Quantitative PCR of primary B cell transcripts from subjects with the minor or major allele at rs1876453 (n=3 per group) was performed using cDNA transcribed using random primers and MultiScribe reverse transcriptase (Applied Biosystems), customized lncRNA 9124 primers and probe that targeted spliced exons, Taqman assays for U6 snRNA and β-actin mRNA, and the Applied Biosystems 7500 Real-Time PCR System. Relative expression levels of lncRNA 9124, normalized to either U6 snRNA (A) or β-actin (B), were calculated using the comparative CT method. Columns and error bars represent the mean+SD for each group. P values were determined using a two-tailed Student t test and a p value of <0.05 was considered significant.

### Example V

### CR1 Ligation Induction of Regulatory B Cells

CR1/CR2 mediates a 2-fold expansion of B10 cells after CFA injection (supra) but the mechanism by which CFA induces suppression of autoimmune diabetes is not fully understood. It is believed to result from the activation of multiple innate immune pathways, including Toll-like receptors (TLRs), Stimulator of Interferon Genes (STING), and Retinoic Acid Inducible Gene-1 (RIG-1)-like receptors. Gulden et al., 2014. “Toll-Like Receptor Activation in Immunity vs. Tolerance in Autoimmune Diabetes” Front Immunol 5: 119-136; Huang et al. 2013. “Cutting edge: DNA sensing via the STING adaptor in myeloid dendritic cells induces potent tolerogenic responses” J Immunol 191: 3509-3513; Sharma et al., 2015. “Suppression of systemic autoimmunity by the innate immune adaptor STING” Proc Natl Acad Sci USA 112: E710-717; and Reikine et al., 2014. “Pattern Recognition and Signaling Mechanisms of RIG-I and MDA5. Front Immunol 5: 342.

In order to dissect the mechanism by which CR1/CR2 participate in regulatory B cell expansion, the effects of immune agonists will be examined for each of these pathways on the induction of B10 cells in CR1/CR2-sufficient and -deficient NOD mice. Furthermore, it will be shown that this phenotype is not restricted to a single animal model of autoimmune disease. Initial experiments in in the (NZBXSWR)F1 (SNF1) model of systemic lupus suggest that innate immune signals will also suppress disease in this model via expansion of B10 cells. Like the NOD model, SNF1 mice develop early immune dysregulation by 4 weeks of age followed by the development of clinical disease months later. Gavalchin et al., 1985. Lupus prone (SWR×NZB) F1 mice produce potentially nephritogenic autoantibodies inherited from the normal SWR parent” J Immunol 134: 885-894; Gavalchin et al., 1987. “The NZB×SWR model of lupus nephritis. II. Autoantibodies deposited in renal lesions show a restricted idiotypic diversity” J. Immunol. 138: 138-148; Datta et al., 1987. “Induction of a cationic shift in IgG anti-DNA autoantibodies. Role of T helper cells with classical and novel phenotypes in three murine models of lupus nephritis” J. Exp. Med. 165: 1252-1268; and Eastcott et al., 1983. “Genetic analysis of the inheritance of B cell hyperactivity in relation to the development of autoantibodies and glomerulonephritis in NZB×SWR crosses” J. Immunol. 131: 2232-2239.

Administration of low doses of nucleosomal peptides generates tolerogenic plasmacytoid dendritic cells that expand autoantigen-specific CD8+ and CD4+CD25+ regulatory T cell subsets and contract inflammatory Th17 cells, thereby suppressing autoantigen-specific Th and B cells, decreasing renal inflammation, and delaying death from severe nephritis. Kang et al., 2005. “Very low-dose tolerance with nucleosomal peptides controls lupus and induces potent regulatory T cell subsets”. J. Immunol. 174: 3247-3255; and Kang et al., 2007. “Low-dose peptide tolerance therapy of lupus generates plasmacytoid dendritic cells that cause expansion of autoantigen-specific regulatory T cells and contraction of inflammatory Th17 cells” J. Immunol. 178: 7849-7858.

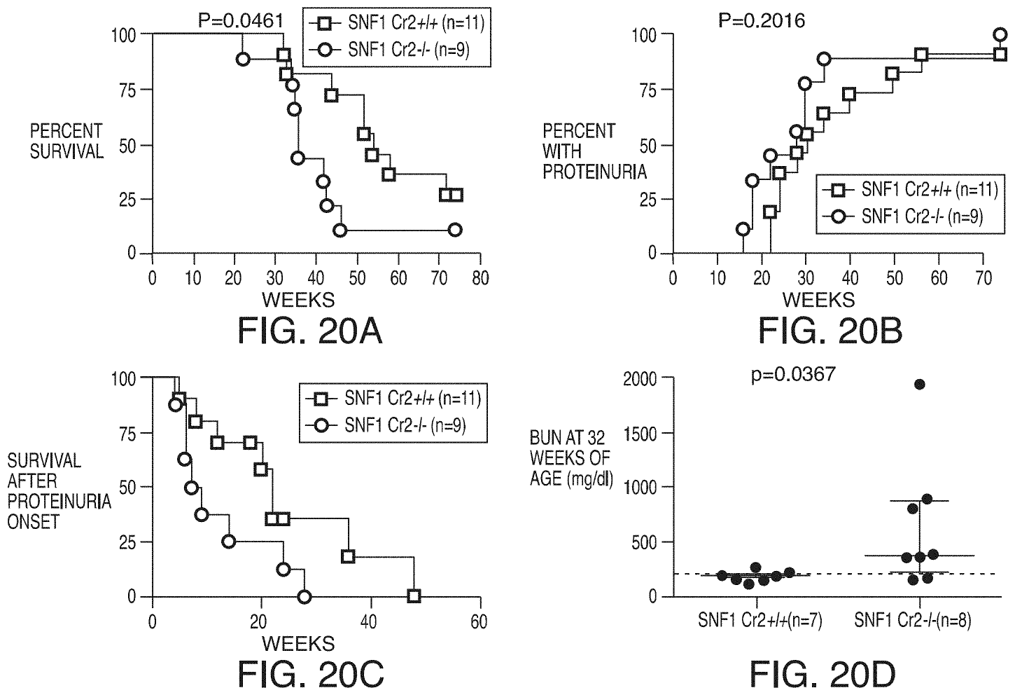

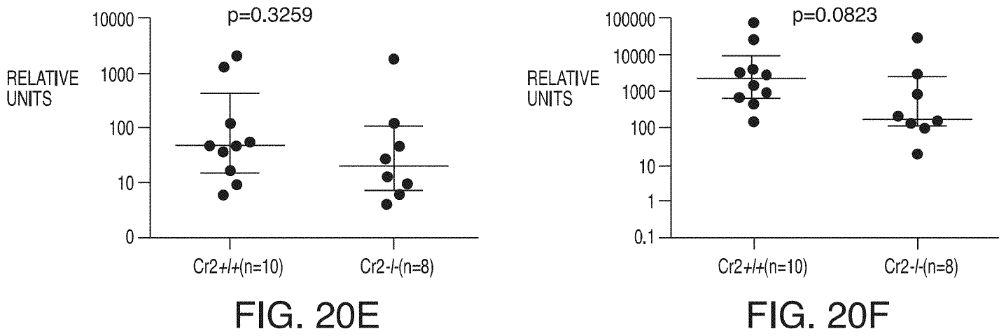

The role of regulatory B cells has not been explored in this model, but our preliminary data suggest that CR1/CR2 deficiency accelerates renal failure and death without altering autoantibody production, suggesting that these receptors protect from disease and could provide additional benefit by augmenting induction of regulatory B cells. FIG. 20. Finally, a protective effect of the CR2 SNP has been identified in lupus and this SNP is associated with increased CR1 transcription (supra), it is possible that lupus protection may be conferred by expansion of regulatory B cells in human peripheral blood mononuclear cell (PBMC) cultures incubated with C3b-anti-BCR tetramers and pro-inflammatory stimuli.

### Example VI

### Differential Expansion of Regulatory B Cells by CR1/CR2

To determine the optimal kinetics for CR1/CR2-mediated expansion of regulatory B cells induced by CFA, CFA injections will be administered at 4, 6, 8, or 10 weeks and spleens harvested at 8, 9, or 10 days after injection. In addition, the mechanism for CFA induction of B10 cells will be evaluated by administering agonists for TLR, STING, and RIG1-like receptor pathways (InvivoGen, San Diego, Calif.) or PBS intravenously at 4, 6, 8, or 10 weeks with spleens harvested 3 and 24 hours after injection. Splenocytes will be incubated with PMA, ionomycin, and monensin for 5 hours, then FcγR blocked with 2.4G2, dead cells excluded with a viability marker, B cells identified with a fluorescent-labeled antibody to CD19, and cells fixed, permeabilized and stained with fluorescent-labeled antibodies to CD19 plus IL10, TGFβ, or IL35 (BioLegend, San Diego, Calif.) or an isotype control for intracellular flow cytometry (33). Absolute number of cytokine-positive B cells in the spleen will be determined using an LSRII flow cytometer (BD Biosciences). The expected standard deviation for each group is 0.32×106 based on our preliminary data, so a sample size of 7 mice per group (CR1/CR2-sufficient or -deficient receiving CFA/agonist or PBS) has 80% power to detect a difference between the means of 0.67×106, which is comparable to the difference we observed (FIG. 8), with a significance level (alpha) of 0.01 (two-tailed). Female mice will be used in these experiments because female NOD and SNF1 mice develop disease earlier and at a higher penetrance. These experiments will be done in both NOD and SNF1 mice; if we identify a CR1/CR2-mediated effect of CFA and/or immune agonists on induction of regulatory B cells in SNF1 mice, we will also include SNF1 mice in the following experiments.

### Example VII

### Regulatory B Cell Induction by CR1/CR2 On

### B Cells or Follicular Dendritic Cells (FDC)

CR1/CR2 are expressed on both B cells and FDC in the mouse, and in order to determine which of these cells induce regulatory B cells, we will perform bone marrow chimeras. Bone marrow will be harvested from the femur and tibia of female donor mice sufficient or deficient for CR1/CR2, embryonic stem cells enriched, and 5×106 cells injected intravenously into lethally irradiated syngenic female mice sufficient or deficient for CR1/CR2. Two weeks after engraftment, CFA/innate immune agonists or PBS will be administered and spleens subsequently harvested for direct identification of regulatory B cells by flow cytometry. 14 female chimeras will be studied for each of the following conditions: Cr2 WT->Cr2 WT, Cr2 KO->Cr2 WT, Cr2 WT->Cr2 KO, and Cr2 KO->Cr2 KO, with 7 mice receiving CFA or agonist and 7 receiving PBS.

### Example VIII

### Intrinsic Versus Extrinsic Effects of B Cell CR1/CR2

It is currently unknown whether CR1/CR2 directly stimulate the expansion of regulatory B cells or induce other cells to stimulate this expansion, whether as a result of cytokine secretion, antigen presentation, or cell contact. To evaluate this, adoptive transfers will be performed of 5×106 Cr2−/− and 5×106 Cr2+/+ splenocytes to 14 sublethally irradiated 4- week-old female mice. After 24 hours, recipients will be treated with CFA/innate immune agonists or PBS and spleens subsequently harvested. Regulatory B cells will be identified by flow cytometry as described above, with their origin determined by concomitant staining with an antibody to CR2 (7E9) that does not bind the C3d binding domain in the event that it is occupied in vivo.

### Example IX

### Cell Subset(s) from which Regulatory B Cells Induced by CR1/CR2 are Derived

Regulatory B cells have been identified in several lineages of murine B cells, including marginal zone (MZ), transitional 2-marginal zone precursor (T2-MZP), and naïve (45), and it is not clear whether they have a common origin or can be induced from multiple cell types. To determine the subset(s) containing regulatory B cells induced by CR1/CR2, B cell subsets from CR1/CR2-sufficient and -deficient mice will be stained after treatment with CFA/immune agonists and determine the numbers of IL10, IL35, and TGFβ-producing cells in each subset, using intracellular cytoplasmic flow cytometry as described above. B cell subsets will be sorted based on expression of CD19, CD5, CD23, CD24, and CD1d [MZ (CD19+CD23-CD24hiCD1dhi), T2-MZP (CD19+CD23hiCD24hiCD1dhi), and B10 (CD19hiCD5+CD1dhi)], as antibodies to CD21 cannot be used in CR1/CR2-deficient mice.

### Example X

### Suppressive Effect of Regulatory B Cells Induced by CFA/Innate Agonists

Adoptive transfers will be performed of 10⁶ B cells purified by positive magnetic selection from CFA/innate agonist-treated CR1/CR2- sufficient or -deficient female mice to sublethally irradiated 4-week-old female NOD or SNF1 mice. NOD mice will be monitored for 30 weeks. Blood glucose will be checked weekly beginning at 10 weeks of age, then daily if blood glucose levels are >200 mg/dl. Three consecutive blood glucose levels>200 mg/dl will be considered as onset of diabetes. The expected proportion of unmanipulated NOD mice without diabetes at 30 weeks of age is 0.15, and a sample size of 20 mice per group will have 80% power to detect an increase in proportion of mice free of diabetes of 0.43 with a significance level (alpha) of 0.05 (two-tailed). SNF1 mice that were adoptively transferred with stimulated B cells at 4 weeks of age will be immunized with 100 μg nephritogenic peptide, a 100 μg control peptide emulsified in CFA, or CFA alone at 12 weeks of age, followed by three booster immunizations at 2-week intervals with 50 μg peptide adsorbed on alum to accelerate disease or alum alone. Kaliyaperumal et al., 2002. “Naturally processed chromatin peptides reveal a major autoepitope that primes pathogenic T and B cells of lupus” J. Immunol. 168:2530-2537.

The mice will be monitored for 28 weeks with weekly urine checks for proteinuria and biweekly serum draws for BUN. The onset of persistent proteinuria (two consecutive readings of >300 mg/dl) will be considered as onset of severe lupus nephritis. At 28 weeks, kidneys will be fixed and frozen for grading of glomerulonephritis and immune complex deposition. The expected proportion of SNF1 mice immunized with nephritogenic peptide without lupus nephritis at 28 weeks of age is 0.01, and a sample size of 10 mice per group will have 80% power to detect an increase in survival proportion of 0.46 with a significance level (alpha) of 0.05 (two-tailed).

### Example XI

### Clinical Functional Study Of Regulatory B Cells

African American men and non-pregnant women will be enrolled in a clinical study, half of whom will be homozygous for the major allele at rs1876453 and half of whom will be heterozygous or homozygous for the minor allele for these studies. Subjects will be between the ages of 18 and 60, on no medications, and without a personal history of chronic illness or family history of autoimmune disease. European-American (EA) and Hispanic (HS) subjects have 20 SNPs in strong or complete LD with rs1876453, whereas African Americans have only a single SNP in complete linkage disequilibrium (LD) with the protective SNP (r561240730, r2 0.81, D′ 1). There is no evidence of open chromatin or histone marks suggestive of a regulatory domain at rs61240730, and therefore we will be able to more easily discriminate the functional effects of the protective CR2 SNP from linked SNPs in this population. Ward et al., 2012. “HaploReg: a resource for exploring chromatin states, conservation, and regulatory motif alterations within sets of genetically linked variants” Nucleic Acids Res 40: D930-934. Based on the minor allele frequency in this population, one hundred fifty eight (158) subjects will be recruited in order to enroll fifty (50).

Subjects will provide 2 milliliters of saliva from which DNA will be extracted for genotyping at rs1876453 using a TaqMan SNP Genotyping Assay (Applied Biosystems). DNA from saliva and blood samples is comparable when genotyping using TaqMan assays, and saliva sampling increases participant recruitment and reduces resources needed for sample collection. Abraham et al., 2012. “Saliva samples are a viable alternative to blood samples as a source of DNA for high throughput genotyping’ BMC Med Genomics 5: 19.

### Example XII

### Induction of Regulatory B Cells by C3b-Anti-BCR Tetramers in Humans with a Protective CR2 SNP

It will be determined whether individuals with the protective CR2 SNP are better able to expand their regulatory B cells with exposure to B cell stimuli provided under inflammatory conditions. Peripheral blood mononuclear cells (PBMCs) will be prepared and incubated with anti-BCR tetramers, C3b-anti-BCR tetramers, C3b-anti-BCR tetramers+factor I, C3dg-anti-BCR tetramers, or media alone with and without anti-CD40, LPS (TLR4 agonist), CpG (TLR9 agonist), cytokines that induce regulatory B cells (IL-21, IL-35), or combinations of these innate and adaptive immune stimulators for 48 hours. In the last 5 hours, cells will be pulsed with PMA and ionomycin to induce IL-10 transcription in B regulatory cells along with Brefeldin A to prevent its secretion, then FcγR blocked with mouse IgG, dead cells excluded with a viability marker, B cells identified with a fluorescent-labeled antibody to CD19, and cells fixed, permeabilized and stained with fluorescent-labeled antibodies to CD19 plus IL10, TGFβ, or IL35 (BioLegend, San Diego, Calif.) or an isotype control for intracellular flow cytometry. Absolute number of cytokine-positive B cells in the spleen will be determined using an LSRII flow cytometer (BD Biosciences). A sample size of 25 per group has 80% power to detect a difference between means of 0.32×106 with a significance level (alpha) of 0.01 (two-tailed). Next, we will determine the specific subsets in which the regulatory B cells induced in association with the protective SNP by staining for transitional (CD19+CD24hiCD38hiCD27−IgMhi), naïve (CD19+CD24intCD38intCD27−IgM+), unswitched memory (CD19+CD24hiCD38-/loCD27+IgM+), and switched memory (CD19+CD24hiCD38−/loCD27+IgM−) prior to fixing and permeabilizing the cells. Subsets will be sorted and tested for their ability to inhibit T cell proliferation in vitro. For these experiments, purified CD4+ T cells will be CF SE-labeled and stimulated with plate-bound CD3 mAb and the effect of activated B cells or their supernatants on T cell proliferation and induction of regulatory T cells will be assessed. Khoder et al., 2014. “Regulatory B cells are enriched within the IgM memory and transitional subsets in healthy donors but are deficient in chronic GVHD” Blood 124: 2034-2045.

### Example XIII

### Generation of Bone Marrow Chimeras

To maintain appropriate cell- and stage-specific expression of the transgenes, which is critical for normal B cell development, we will clone the Cr2 minimal promoter and its associated enhancer elements into a lentiviral vector, replacing the endogenous promoter. Marchbank et al., 2002. “Expression of human complement receptor type 2 (CD21) in mice during early B cell development results in a reduction in mature B cells and hypogammaglobulinemia” J. Immunol. 169: 3526-3535; Hu et al., 1997. “Expression of the murine CD21 gene is regulated by promoter and intronic sequences” J. Immunol. 158:4758-4768; and Zabel et al., 2000. “Cell-specific expression of the murine CD21 gene depends on accessibility of promoter and intronic elements” J. Immunol. 165: 4437-4445.

Next, cDNA is cloned for the monomeric fluorescent proteins, eGFP or mCherry, into the vector. Finally, cDNA from mouse CR1 is cloned into the eGFP-containing vector and the cDNA from mouse CR2 into the mCherry-containing vector. The vectors will be packaged into virus and lentivirus will be produced for transduction of hematopoietic stem cells (HSC).

Bone marrow will be harvested from the femur and tibia of female donor Cr2−/− and Cr2+/+ mice, and HSC enriched and transduced with lentivirus. Transduced HSCs will then injected into lethally irradiated female Cr2+/+ recipients. Experimental groups will include: CR1-transduced Cr2+/+ HSC, CR2-transduced Cr2+/+ HSC, CR1/CR2-transduced Cr2+/+ HSC, empty vector-transduced Cr2+/+ HSC, CR1-transduced Cr2−/− HSC, CR2-transduced Cr2−/− HSC, CR1/CR2-transduced Cr2−/− HSC, empty vector transduced Cr2−/− HSC. Mice from both the NOD and SNF1 backgrounds will be studied in two separate experiments. Sample sizes will be 20 mice per group for NOD and 10 mice per group for SNF1.

### Example IVX

### Induction of Regulatory B Cells and Monitoring of Disease

Two weeks after bone marrow engraftment prepared in accordance with Example XIII, CFA/innate immune agonists or PBS will be administered to mice, using the treatment regimen that maximally induced regulatory B cells. SNF1 mice will additionally be immunized with nephritogenic peptide, control peptide, or CFA at 12 weeks followed by 3 booster immunizations at two-week intervals. NOD chimeras will be monitored for 30 weeks for development of diabetes and SNF1 chimeras will be monitored for 28 weeks for development of lupus nephritis.

### Example XV

### Definition of CR2 lncRNA 5′ and 3′ Ends

Rapid amplification of cDNA ends (RACE) will be used to identify the 5′ and 3′ ends of the transcript (FirstChoice RLMRACE Kit, Ambion). To confirm that the 5′ RACE is detecting the noncoding RNA and not pre-mRNA, a gene-specific primer is used that flanks the splice site in the transcript. RACE will be performed initially in primary B cells and subsequently in other tissues in which the lncRNA is identified

### Example XVI

### Determination of CR2 lncRNA Intracellular Localization

To identify the intracellular localization of the lncRNA, the nuclear and cytosolic compartments will be fractionated before preparing RNA, and perform quantitative rtPCR on both fractions.

### Example XVII

### Tissue Distribution of CR2 lncRNA

To determine tissue distribution, T cells, B cells, monocytes, and neutrophils are sorted from peripheral blood, prepare RNA, and perform quantitative rtPCR on RNA from the cell subsets. RNA is prepared from sorted transitional, naïve, and class-switched and unswitched memory B cells to identify whether its expression is restricted to specific developmental stages. Published RNA-Seq datasets will be examined to determine whether the lncRNA has been identified in other tissues besides peripheral blood.

### Example XVIII

### Effects of Activating Cell Signals on the CR2 lncRNA

B cells purified from peripheral blood will be incubated with model immune complexes comprised of streptavidin tetramers containing biotinylated C3b and biotinylated polyclonal antibody to the B cell receptor (BCR) in the presence of factor I to initiate cleavage of C3b to iC3b and C3d. These complexes, which would be expected to coligate both CR1 and CR2 with the BCR, mimic the C3b-opsonized dsDNA-anti-dsDNA immune complexes generated prior to disease onset or flare. Cells will also be exposed to cytokines found in the blood of patients with active disease, including IFNα, TNFα, IL6, and BAFF, as well as anti-CD40 and TLR7 and TLR9 agonists. Jacob et al., 2011. “Cytokine disturbances in systemic lupus erythematosus” Arthritis Res Ther 2011: 228. RNA will be extracted from cells after 3, 9, and 24 hours and both qualitative and quantitative rtPCR performed.

### Example XIX

### CR2 lncRNA Alteration of CR1 Transcription

In order to show that the lncRNA is functional and modifies CR1 transcription, siRNA-knockdown techniques will be used with constructs in a measles virus-lentiviral vector, which has been shown to efficiently transduce quiescent B cells without activation, cell-cycle entry, or phenotypic switch. Frecha et al., 2010. “Advances in the field of lentivector-based transduction of T and B lymphocytes for gene therapy” Mol Ther 18:1748-1757. The siRNA will be directed towards the portion of the lncRNA that corresponds to the intronic region of the CR2 gene in order to avoid depletion of CR2 mRNA. If an effect is seen, confirmation will determine that it is specific for CR1 by overexpressing the lncRNA and determining whether this is associated with a corresponding increase in CR1 transcription.

As an alternative approach and also to identify its other genomic targets, ChIRP-Seq is used with tiling anti-sense DNA oligonucleotides to capture and purify lncRNA-chromatin complexes from the cell. Chu et. al., 2011. “Genomic maps of long noncoding RNA occupancy reveal principles of RNA-chromatin interactions” Mol Cell 44: 667-678. Primary B cells from 3 individuals homozygous for the major allele at rs1876453 and 3 individuals homozygous or heterozygous for the minor allele will be crosslinked in vivo, and their chromatin extracted and homogenized. Biotinylated complementary oligonucleotides that tile the spliced noncoding CR2 transcript will be hybridized to the target RNA and isolated using magnetic streptavidin beads. DNA will be eluted from the copurified chromatin and subjected to high-throughput sequencing. Negative controls will include non-interacting control probes and RNAse treatment of lysate prior to ChIRP.

### Example XX

### Protein Partners of CR2 lncRNA

A comprehensive identification of RNA binding proteins will be performed by mass spectrometry (ChIRP-MS) to identify the RNA binding proteins associated with the CR2 lncRNA Chu et al., 2015. “Systematic discovery of Xist RNA binding proteins” Cell 161: 404-416.

Primary B cells from 3 individuals homozygous for the major allele at rs1876453 and 3 individuals homozygous or heterozygous for the minor allele will be extensively crosslinked in vivo with formaldehyde, target RNA retrieved with oligonucleotide hybridization, and gentle biotin elution used to release associated proteins. Enriched proteins will be identified by liquid chromatography-tandem mass spectrometry (LC-MS/MS). Negative controls will include non-interacting control probes and RNAse treatment of lysate prior to ChIRP. Mass spectrometry will be performed.

Protein partners will be validated by immunoblotting. Functional effects of the protein-lncRNA interaction will be confirmed by knocking down the protein using siRNA and then evaluating transcriptional effects by quantitative PCR, epigenetic marks by combined immunofluorescence and RNA FISH, and direct protein-RNA interactions by UV-cross-linking RNP immunoprecipitation followed by RT-PCR.

### Example XXI

### Clinical RNA-Seq Study

The study will comprise African-American (AA) subjects with lupus, as wells as their first-degree relatives (FDRs).

FDRs will provide 2 milliliters of saliva from which DNA will be extracted for genotyping at rs1876453 using a TaqMan SNP Genotyping Assay (Applied Biosystems). DNA from saliva and blood samples is comparable when genotyping using TaqMan assays, and saliva sampling can increase participant recruitment and reduce resources required for sample collection. Abraham et al., 2012. “Saliva samples are a viable alternative to blood samples as a source of DNA for high throughput genotyping” BMC Med Genomics 5:19. Each proband will contribute an average of 3 FDR, resulting in enrollment of 180 FDR (Table 1, line 1), 29 of whom will have one or two copies of the minor allele at rs1876453 and 150 of whom will have two copies of the major allele. Sex-matched pairs with and without the minor allele at rs1876453 from the same lupus proband will be identified for further study; they will also be matched as closely as possible with respect to age. Although only 1 subject is expected to be homozygous for the minor allele at rs1876453 (Table 1, line 1), the data presented herein suggest that the minor allele has a dominant effect, so comparison of FDRs heterozygous for the minor allele with their homozygous major family member should still be informative.

AA subjects for the genomics studies are expected to have only a single SNP in complete linkage disequilibrium (LD) with the protective SNP (r561240730, r2 0.81, D′ 1), whereas European-American (EA) and Hispanic (HS) subjects have 20 SNPs in strong or complete LD with the SNP. There is no evidence of open chromatin or histone marks suggestive of a regulatory domain at rs61240730 (62), and therefore we will be able to more easily discriminate the functional effects of the protective CR2 SNP from linked SNPs in this population. Ward et al., 2012. “HaploReg: a resource for exploring chromatin states, conservation, and regulatory motif alterations within sets of genetically linked variants” Nucleic Acids Res 40:D930-934.

We will include lupus patients who meet the revised 1997 American College of Rheumatology (ACR) criteria for the diagnosis of SLE, and their male and non-pregnant female FDR between the ages of 18 and 60. This age range was selected to increase the likelihood that we can recruit sufficient numbers of FDRs for analysis; we will age-match FDRs for each lupus proband whenever possible and include age as a covariate in our analyses. We will exclude subjects who have a positive CSQ suggestive of a possible or probable connective tissue disease or who have evidence of another autoimmune disease. We will also exclude subjects who have a personal history of malignancy, chronic infections, or other chronic disease for which the subject is taking medications that could modulate their immune responses.

TABLE 1
Anticipated Enrollment of FDRs with Variation at rs1876453

| | Enrollment | Enrollment | MM | MM | Mm | Mm | mm | mm |
|---|---|---|---|---|---|---|---|---|
| | SLE | FDR | Frequency | Enrolled | Frequency | Enrolled | Frequency | Enrolled |
| AA | 60 | 180 | 0.836 | 150 | 0.156 | 28 | 0.008 | 1 |
| (RNA-Seq) | | | | | | | | |
| AA | 80 | 240 | 0.836 | 201 | 0.156 | 37 | 0.008 | 2 |
| (Validation) | | | | | | | | |
| EA | 80 | 240 | 0.834 | 200 | 0.156 | 37 | 0.01 | 2 |
| (Validation) | | | | | | | | |

EA = European American;
AA = African American;
MM = major allele homazygate;
Mm = heterozygote;
mm = minor allele homozygote

### Sample Size and Power Analysis

We will identify 15 matched FDR pairs to undergo RNA-Seq. We chose this sample size because we believe that it is feasible to match FDRs homozygous with the major allele with at least 15 of the 29 FDRs who will have at least one copy of the minor allele. (Table 1, line 1).

The power analysis using RNASeqPower (63) for a two-group comparison indicates that there is sufficient power (>75%) to detect effect sizes of at least 2.0 for expression between the two groups with a sample size of 15, average depth per gene of 10-20 and coefficient of variation for biological replicates of 0.20, values common in human studies (63). Hart et al., 2013. “Calculating sample size estimates for RNA sequencing data” J Comput Biol 20: 970-978; and Table 2.

TABLE 2
Power for Paired Analysis of 15 FDR Pairs

| Average Depth | Coefficient | | |
|---|---|---|---|
| Per Gene | of Variation | Effect Size | Power |
| 10 | 0.2 | 1.75 | 0.5160894 |
| 20 | 0.2 | 1.75 | 0.8538164 |
| 10 | 0.35 | 1.75 | 0.2099534 |
| 20 | 0.35 | 1.75 | 0.3573207 |
| 10 | 0.2 | 2 | 0.8455876 |
| 20 | 0.2 | 2 | 0.9884541 |
| 10 | 0.35 | 2 | 0.4875071 |
| 20 | 0.35 | 2 | 0.6966711 |

The alpha level is set at 5e-05, which corresponds to a conservative Bonferroni correction for multiple comparisons that would result in at most one false positive statement for the 20,000 tests for each gene. These are also conservative calculations because the RNASeqPower package assumes independent observations that are not paired as in the proposed study; therefore, the power is also underestimated. We use these conservative choices as there are limited alternatives for power analysis for RNA-seq studies without existing data.

### Preparation of B Cell Subsets

Each pair of matched FDR for each lupus proband will be scheduled for a study visit at which 200 milliliters of peripheral blood will be collected by standard phlebotomy. Collection of up to 550 ml of blood for research purposes over an 8-week period from healthy nonpregnant adults who weigh at least 110 pounds is considered to present no more than minimal risk to human subjects, according to the Office for Human Research Protections at the U.S. Department of Health and Human Services. Because B cells comprise 5-10% of the total mononuclear cell population, 200 ml of blood should provide sufficient numbers of cells for the proposed studies. Mononuclear cells will be recovered by Ficoll-Paque Plus (GE Health Care, Pittsburgh, Pa.) and B cells purified by negative selection using immunomagnetic cell isolation (Stem Cell Technologies), immediately stained with directly labeled antibodies to CD20 and CD27, and sorted using a FACSAria flow cytometer (BD Biosciences). Each subpopulation of transitional/naïve (CD20+CD27−) and memory (CD20+CD27+) B cells will be sorted directly into complete media and either used immediately or frozen for subsequent analyses. Evaluation of the B cell transcriptome to identify differential gene expression associated with the minor allele at rs1876453. Illumina HiSeq cDNA libraries will be prepared using fragmented ribosomal RNA-depleted RNA from transitional/naïve and memory B cells. Following validation and normalization, cDNA libraries will be subjected to paired-end sequencing on an Illumina HiSeq2000 platform. To maximize coverage with inclusion of low-abundance transcripts, each lane will contain 4 libraries for a read depth of 75 million pair-end reads of 100 nucleotides.

### Example XXII

### Autoantibody Analysis

Serum will be prepared from the blood of all FDR pairs upon enrollment and tested for ANA by indirect immunofluorescence on HEp-2 substrates beginning at a 1:80 dilution with 1:2 serial dilutions until reactivity is no longer noted. The final dilution at which reactivity is noted, the intensity of staining at that dilution, and the pattern of staining will be recorded. All samples positive at a 1:80 dilution will be tested for anti-dsDNA by ELISA, and for anti-Sm, anti-SSA, anti-SSB, and anti-RNP by immunodiffusion. Autoantibody testing will be performed in the Clinical Rheumatology Laboratory at the University of Colorado Denver, which is certified to test human specimens in accordance with the Clinical Laboratory Improvement Amendments of 1988 (CLIA). At a 1:80 dilution, ˜⅓ of healthy controls test positive for ANA in this laboratory, whereas at 1:320, only ˜5-7% are positive. Autoantibody status will be included as a covariate in the final analyses.

### Example XXIII

### Genome-Wide Effects SNPs on DNA Accessibility and Transcription Factor Binding at Regulatory Domains

The assay for transposase-accessible chromatin using sequencing (ATAC-Seq) will be performed to identify regions of increased chromatin accessibility and to map regions of transcription-factor binding and nucleosome position. Buenrostro et al., 2013. “Transposition of native chromatin for fast and sensitive epigenomic profiling of open chromatin, DNA-binding proteins and nucleosome position” Nat Methods 10: 1213-1218. It uses hyperactive Tn5 transposase to simultaneously cut and ligate adapters for high-throughput sequencing at regions of increased accessibility

Nuclei will be prepared, the transposition reaction will be carried out, and library fragments will be amplified by PCR and purified. To stop amplification before saturation and therefore reduce GC and size bias in the PCR, we will monitor the PCR reaction using quantitative PCR. Following validation and normalization, DNA libraries will be sequenced with 50 bp, single end reads on an Illumina HiSeq platform, with each lane containing 3 libraries. Reads will be aligned to the human genome using Bowtie. Trapnell et al., 2010. “Transcript assembly and quantification by RNA-Seq reveals unannotated transcripts and isoform switching during cell differentiation” Nat Biotechnol 28:511-515.

For peak-calling and footprinting, the read start sites will be adjusted to represent the center of the transposon binding event. ZINBA will be used to call ATAC-Seq peaks. Rashid et al., 2011. “ZINBA integrates local covariates with DNAseq data to identify broad and narrow regions of enrichment, even within amplified genomic regions” Genome Biol 12: R67. Enriched reads will be identified as those with a posterior probability of >0.8. DNA footprinting will be performed using CENTIPEDE. Pique-Regi et al., 2011. “Accurate inference of transcription factor binding from DNA sequence and chromatin accessibility data” Genome Res 21:447-455.

Transcription factor regulatory networks will be constructed by comparing the GENCODE v.14 genes with the genome-wide set of posterior probabilities estimated by CENTIPEDE. Transcription factors that are predicted to interact differentially with regulatory domains as a result of the protective CR2 SNP will be validated using ChIP-PCR in age- and sex-matched cohorts consisting of 25 subjects homozygous for the major allele and 25 subjects homozygous or heterozygous for the minor allele from each of the three ethnic groups (EA, AA, HS).

### Example IVXX

### Genome-Wide Effects of the SNP on Enhancer-Associated Histone Marks

Chromatin immunoprecipitation (ChIP) followed by high-throughput DNA Sequencing (ChIP-Seq) will be performed using EMD Millipore ChIP assay kits and following ENCODE guidelines. Landt et al., 2012. “ChIP-seq guidelines and practices of the ENCODE and modENCODE consortia” Genome Res 22: 1813-1831. Briefly, naïve and memory peripheral blood B cells from 25 subjects homozygous for the major allele at rs1876453 and subjects homozygous or heterozygous for the minor allele will be treated with formaldehyde to cross-link proteins covalent to DNA, followed by cell disruption and sonication to shear the chromatin to a target size of 100-300 base pairs. The H3K27ac, H3K4me1, and H3K4me2 histone modifications, which mark active enhancers, will be enriched with their bound DNA by purification with antibodies specific for the factors. Cross-links will be reversed, enriched DNA purified, and Illumina HiSeq libraries prepared for analysis by high-throughput DNA sequencing. DNA libraries will be sequenced with 50 bp, single-end reads on an Illumina HiSeq platform, with each lane containing 3 libraries. ChIPseq data will be mapped to the genome with GSNAP, and peaks will be called using HOMER homer.salk.edu/homer/ngs/index.html). HOMER will also be used to annotate and find motifs within peaks. Validation of differentially modified histones based on the allele at rs1876453 will be confirmed using ChIPPCR in age- and sex-matched cohorts including subjects homozygous for the major allele and subjects homozygous or heterozygous for the minor allele from each of the three ethnic groups (EA, AA, HS).

### Example XXV

### Regulatory Domains that are Modified by the SNP for CR1 Enhancer Activity

Once putative enhancer domains are identified that are modified differentially by the SNP, they will be assessed for enhancer activity using a luciferase reporter vector with a weak SV40 promoter. Subsequently, they are cloned into a luciferase reporter vector containing −376/+77 or −1786/+77 of the CR1 promoter. Enhancer domains will be cloned into promoter-less vectors as well to determine the presence of intrinsic promoter activity in these domains. Site directed mutagenesis will be performed to introduce the protective minor allele into the vectors containing the putative enhancer from CR2 intron 1. Enhancer domains will be examined upstream and downstream of the promoter and in both orientations. Each clone will be cotransfected with a Renilla luciferase control plasmid into CR1-expressing B cell lines for normalization. The basal CR1 promoter is believed to be located 376 nucleotides upstream of the transcription initiation site, and there is evidence that a repressive element lies between −487 and −376. Kim et al., 1999. “Characterization of the human CR1 gene promoter. Biochem Mol Biol Int 47: 655-663; and Funkhouser et al., 1999. “Complement receptor type 1 gene regulation: retinoic acid and cytosine arabinoside increase CR1 expression” Scand J Immunol 49: 21-28.

### Example XXVI

Genome-Wide Effects of SNPs on Long Noncoding RNA Transcription and their Coding Gene Targets

Illumina HiSeq cDNA libraries will be prepared using fragmented ribosomal RNA-depleted RNA from naïve and memory B cells. Following validation and normalization, cDNA libraries will be subjected to paired end sequencing on an Illumina HiSeq platform. To maximize coverage with inclusion of low-abundance transcripts, each lane will contain 4 libraries for a read depth of 75 million pair-end reads of 100 nucleotides.

For mRNA and lncRNA profiling, transcript levels will be quantified in fragments per kilobase of exon per million mapped reads (FPKM). The FPKM reflects the molar concentration of a transcript in the starting sample by normalizing for gene length and for the total read number in the measurement. This allows for comparison of transcript levels both within and between experiments. The computational pipeline consisting of the opensource Tuxedo Suite comprising Bowtie, TopHat, and Cufflinks are typically used for alignment and discovery of differential gene expression. Trapnell et al., “Transcript assembly and quantification by RNA-Seq reveals unannotated transcripts and isoform switching during cell differentiation” Nat Biotechnol 28:511-515.

Each read generated by each sample will be mapped to the human genome by Bowtie. Subsequently, TopHat will analyze these mapped locations and assign them their gene/splice variant of origin. The third program of the series, Cufflinks, calculates the prevalence of transcripts from each known gene based on normalized read counts. From this we can determine significant isoform expression among the genotypic groups using ANOVA in R, utilizing a false discovery rate of 0.05 and collapsing the heterozygote and homozygote minor allele groups. Simulation studies has found a hybrid pipeline of gSNAP coupled with Cufflinks to be equally powerful in detecting transcription differences while minimizing false positives to ˜1% versus ˜10% for Bowtie/Tophat (unpublished data, Jones, K. L.). Thus, a custom pipeline is now used including, but not limited to, gSNAP/Cufflinks/R for RNA-Seq profiling. When the final gene list is formed, Ingenuity Pathway Analysis will be used to identify pathways of interest that may be modified due to the influence of the allele at rs1876453. Differentially expressed transcripts will be validated by quantitative RT-PCR using commercial or custom-designed gene-specific assays and the comparative Ct method. Schmittgen et al., 2008. “Analyzing real-time PCR data by the comparative CT method. Nat Protoc 3:1101-1108.

RNA for validation experiments will be prepared from independent cohorts including subjects homozygous for the major allele and subjects heterozygous or homozygous for the minor allele from each of the three ethnic groups (EA, AA, HS). Flow cytometry, immunoblotting, and immunofluorescence microscopy will be performed to validate differences in protein expression that are suggested by experimental findings from RNA-Seq, using appropriate monoclonal and polyclonal antibodies and standard protocols.

### Example XXVII

### Genome-Wide Effects of SNPs on Transcription of Enhancer RNA Induced by Factors that Drive Regulatory B Cell Development

A subset of the transitional/naïve and memory B cells that are collected will be incubated with at least one perturbation that optimally induces regulatory B cells (i.e., for example, IL-21+anti-CD40) or media alone for 1 hour (49), after which nuclei will be prepared and nuclear run-on assays performed to extend nascent RNAs that are associated with transcriptionally engaged polymerases under conditions where new initiation is prohibited. Yoshizaki et al., 2012. “Regulatory B cells control T-cell autoimmunity through IL-21-dependent cognate interactions” Nature 491: 264-268.

Nascent RNAs will be tagged with the ribonucleotide analog (for example, 5-bromouridine 5′-triphosphate (BrUTP)), chemically hydrolyzed into ˜150 bp fragments, and immunopurified using anti-BrU beads. cDNA libraries will be prepared and subjected to single-end sequencing on an Illumina HiSeq platform. Each lane will contain 3 libraries for a read depth of 100 million single-end reads of 50 nucleotides. Read mapping, transcriptome reconstruction (68), and expression quantification will be performed. Azofeifa et al., 2014. “FStitch: A fast and simple algorithm for detecting nascent RNA transcripts” 5th ACM Conference on Bioinformatics, Computational Biology and Health Informatics.

1. A viral expression vector comprising a human DNA sequence encoding a human long noncoding RNA (lncRNA) molecule and a fragment from intron 1 of a complement receptor 2 (CR2) gene.

2. The viral expression vector of claim 1, wherein the fragment from intron 1 of a CR2 gene further comprises a single nucleotide polymorphism (SNP).

3. The viral expression vector of claim 1, wherein the viral expression vector further comprises an inducible expression element.

4. The viral expression vector of claim 3, wherein said inducible expression element is reversible.

5. The viral expression vector of claim 1, wherein said viral expression vector is selected from the group consisting of a retroviral vector, an adenoviral vector, a herpes viral vector and a lentiviral vector.

6. The viral expression vector of claim 1, wherein the fragment from intron 1 of a CR2 gene further comprises the sequence gcgagacggtaggg (SEQ ID NO: 13).

7. The viral expression vector of claim 1, wherein the encoded human lncRNA molecule further comprises a sequence of gcgagacggtaggg (SEQ ID NO:13).

8. The viral expression vector of claim 2, wherein the single nucleotide polymorphism (SNP) is a g→a transposition at the fragment from intron 1 of a CR2 gene that corresponds to chromosomal position rs1876453.

9. The viral expression vector of claim 1, wherein the human lncRNA molecule is further encoded by a second exon including a fragment from exon 2 of the CR2 gene.

10. The viral expression vector of claim 1, wherein the sequence of the lncRNA molecule is selected from the group consisting of SEQ ID NOs: 1-8.

11. The viral expression vector of claim 1, wherein the sequence of the lncRNA molecule comprises SEQ ID NO: 14.

12. A method for using a viral expression vector for treating a health condition comprising, administering a composition comprising a viral expression vector comprising a human DNA sequence encoding a human long noncoding RNA (lncRNA) molecule and a fragment from intron 1 of a complement receptor 2 (CR2) gene; and a pharmaceutically acceptable excipient, to a subject having a health condition and treating the health condition.

13. The method of claim 12, wherein the health condition comprises an inflammatory condition.


SEQUENCE LISTING (32 sequences)

SEQ ID NO: 1 - DNA, 536 bp, Artificial Sequence
  Synthetic
cccactggcccctccgggagctgggacctccagaattggaggctgcgccacagaggggcgctgtctggtcggcccggtgtggctggcggcgtgcgggtgctcgcggtctcctgccgggctccccgcccccaggattctgcaggtgctcatcgctctctggccggcggtcagcgctaccgctcgccacgggaaacctacccactgcatgaggcaccagctagccacgtgaggctgtttctgtacacccgaaccgcgctcttgcccagcaaaagcggcagaacctcggctcccttccctaaataagaggatttcttgtggctctcctccgcctatcctaaatggccggattagttattattctacccccattgctgttggtaccgtgataaggtacagttgttcaggtaccttccgcctcattggagaaaaaagtctattatgcataactaaagacaaagtggatggaacctgggataaacctgctcctaaatgtgaatatttcaataaatattcttcttgccctgagcccatagtac

SEQ ID NO: 2 - DNA, 512 bp, Artificial Sequence
  Synthetic
gctgtttctgtacacccgaaccgcgctcttgcccagcaaaagcggcagaacctcggctcccttccctaaataagagtaagacctggttcccaagcatgggtctcagattttctgaactctaatgtgatattggtagagatccgaaacattaaccatccgtagttatttttgagacatggattaaaactattgttttcttcttgttattaacctactgtaatggtgtggacgtccgcaaaggtctcattcaccgtttattatatcttttttttcctccagattggcttgattttaaagaaagattataattaagattagcgtctcttggctacaggatgggtctaagtttctaattatctccacggtaccgtgcattcctagtttataagacgtttactgtgtgcagacctcaataactgggtgacttttggcaagtcatttagctccctgatcttcagtttcttcatctgtaaaatgaaactagtgattaactggcctccaaaaaaaaaaaa

SEQ ID NO: 3 - DNA, 296 bp, Artificial Sequence
  Synthetic
gtttctgtacacccgaaccgcgctcttgcccagcaaaagcggcagaacctcggctcccttccctaaataagagtaagacctggttcccaagcatgggtctcagattttctgaactctaatgtgatattggtagagatcctaaacattaaccatccgtagttatttttgagacatggattaaaactattgttttcttcttgttattaacctactgtaatggtgtggacgtccgcaaaggtctcattcaccgtttattatatctttctttaaaatcaagccaatctggaggaaaaaaa

SEQ ID NO: 4 - DNA, 299 bp, Artificial Sequence
  Synthetic
gctgtttctgtacacccgaaccgcgctcttgcccagcaaaagcggcagaacctcggctcccttccctaaataagagtaagacctggttcccaagcatgggtatcagattttctgaactctaatgtgatattggtagagatcctaaacattaaccatccgtagttatttttgagacatggattaaaactattgttttcttcttgttattaacctactgtaatggtgtggacgtccgcaaaggtttcattcaccgtttattatatctttctttaaaatcaagccaatggggaggaaaaaaa

SEQ ID NO: 5 - DNA, 1433 bp, Artificial Sequence
  Synthetic
ctgtacacccgaaccgcgctcttgcccagcaaaagcggcagaacctcggctcccttccctaaataagagtaagacctggttcccaagcatgggtctcagattttctgaactctaatgtgatattggtagagatcctaaacattaaccatccgtagttatttttgagacatggattaaaactattgttttcttcttgttattaacctactgtaatggtgtggacgtccgcaaaggtctcattcaccgtttattatatctttttttcctccagattggcttgattttaaagaaagattataattaagattagcgtctcttggctacaggatgggtctaagtttctaattatctccacggtaccgtgcattcctagtttataagacgtttactgtgtgcagacctcaataactgggtgacttttggcaagtcatttagctccctgatcttcagtttcttcatctgtaaaatgaaactagtgattaactggccttccacctcactgagctgccctgagcacagatgaaactaagcaaagaaagttggatgttagtcgttactactaaatatcagtaataagcctcctgtacagacttgattttatggagccagcatttaagctaacaagctgggtgtccaattgtttctatggacatccagcttattgaggtgtctggaatacagcacatattagtttattacaactttttgctttcctgacattttactgtctcctgacccttaaacctttctccagaccattatcttcatctcttttctttttctgcatattaactccctttgaacaaatgtatattcacactttttctaagagcagagtacgtccctgacttctcacctatttctaaatttcaaagaggatattatcaactgaaaatcaaattcttcaccagtcctgccaattcagggccacagcagcagcatcatgtaggtcacttaactggagccccatgaaataccaggtctacctatcagccattcaagctactgtgcttctgttgtttgtccatttctcatttttgtaccagtattcccatcctaagcattataattacctgtgtaacaacctcatctcttttaagagattttggttcttatgattggaaaggttaaagagtgacctataggtcactttccaattatgaaaacaaaaaattaagaaatatatatattttcattatttcactccattgttcaaaaatctaaaaggcctccagtggaagaaaatctggtgtcttcagcctttctagccctttggaatgggatctcaccgaagctcacaccccctgatttctccagctgcctgtcttgtatgcgctgctcatggtcttccccacctgtctcccatggctcatgctttgcccactctccactgctaacttttactgctactgcatgctctccaaaaaaaaaaa

SEQ ID NO: 6 - DNA, 1440 bp, Artificial Sequence
  Synthetic
gctgtttctgtacacccgaaccgcgctcttgcccagcaaaagcggcagaacctcggctcccttccctaaataagagtaagacctggttcccaagcatgggtctcagattttctgaactctaatgtgatattggtagagatcctaaacattaaccatccgtagttatttttgagacatggattaaaactattgttttcttcttgttattaacctactgtaatggtgtggacgtccgcaaaggtctcattcaccgtttattatatctttttttcctccagattggcttgattttaaagaaagattataattaagattagcgtctcttggctacaggatgggtctaagtttctaattatctccacggtaccgtgcattcctagtttataagacgtttactgtgtgcagacctcaataactgggtgacttttggcaagtcatttagctccctgatcttcagtttcttcatctgtaaaatgaaactagtgattaactggccttccacctcactgagctgccctgagcacagatgaaactaagcaaagaaagttggatgttagtcgttactactaaatatcagtaataagcctcctgtacagacttgattttatggagccagcatttaagctaacaagctgggtgtccaattgtttctatggacatccagcttattgaggtgtctggaatacagcacatattagtttattacaactttttgctttcctgacattttactgtctcctgacccttaaacctttctccagaccattatcttcatctcttttctttttctgcatattaactccctttgaacaaatgtatattcacactttttctaagagcagagtacgtccctgacttctcacctatttctaaatttcaaagaggatattatcaactgaaaatcaaattcttcaccagtcctgccaattcagggccacagcagcagcatcatgtaggtcacttaactggagccccatgaaataccaggtctacctatcagccattcaagctactgtgcttctgttgtttgtccatttctcatttttgtaccagtattcccatcctaagcattataattacctgtgtaacaacctcatctcttttaagagattttggttcttatgattggaaaggttaaagagtgacctataggtcactttccaattatgaaaacaaaaaattaagaaatatatatattttcattatttcactccattgttcaaaaatctaaaaggcctccagtggaagaaaatctggtgtcttcagcctttctagccctttggaatgggatctcaccgaagctcacaccccctgatttctccagctgcctgtcttgtatgcgctgctcatggtcttccccacctgtctcccatggctcatgctttgcccactctccactgctaacttttactgctactgcatgctctccaaaaaaaaaaa

SEQ ID NO: 7 - DNA, 541 bp, Artificial Sequence
  Synthetic
gctgtttctgtacacccgaaccgcgctcttgcccagcaaaagcggcagaacctcggctcccttccctaaataagagtaagacctggttcccaagcatgggtctcagattttctgaactctaatgtgatattggtagagatcctaaacattaaccatccgtagttatttttgagacatggattaaaactattgttttcttcttgttattaacctactgtaatggtgtggacgtccgcaaaggtctcattcaccgtttattatatctttttttcctccagattggcttgattttaaagaaagattataattaagattagcgtctcttggctacaggatgggtctaagtttctaattatctccacggtaccgtgcattcctagtttataagacgtttactgtgtgcagacctcaataactgggtgacttttggcaagtcatttagctccctgatcttacagatgaagaaactgaagaaacaaaaacttatggcacgggagtaaattcagcattaaaataaatgtaattaaaagaaaaaaaaaaa

SEQ ID NO: 8 - DNA, 507 bp, Artificial Sequence
  Synthetic
gctgtttctgtacacccgaaccgcgctcttgcccagcaaaagcggcagaacctcggctcccttccctaaataagagtaagacctggttcccaagcatgggtctcagattttctgaactctaatgtgatattggtagagatcctaaacattaaccatccgtagttatttttgagacatggattaaaactattgttttcttcttgttattaacctactgtaatggtgtggacgtccgcaaaggtctcattcaccgtttattatatctttttttcctccagattggcttgattttaaagaaagattataattaagattagcgtctcttggttacaggatgggtctaagtttctaattatctccacggtaccgtgcattcctagtttataagacgtttactgtgtgcagacctcaataactgggtgacttttggcaagtcatttagctccctgatcttcagtttcttcatctgtaaaatgaaactagtgattaactggccaaaaaaaaaaa

SEQ ID NO: 9 - DNA, 194 bp, Artificial Sequence
  Synthetic
tgaggctgtttctgtacacccgaaccgcgctcttgcccagcaaaagcggcagaacctcggctcccttccctaaataagaggatttcttgtggctctcctccgcctatcctaaatggccggattagttattattctacccccattgctgttggtaccgtgataaggtacagttgttcaggtaccttccgcctcat

SEQ ID NO: 10 - DNA, 194 bp, Artificial Sequence
  Synthetic
  misc_feature (1)..(5): n is a, c, g, or t
  misc_feature (8)..(24): n is a, c, g, or t
  misc_feature (38)..(39): n is a, c, g, or t
  misc_feature (44)..(45): n is a, c, g, or t
  misc_feature (54)..(54): n is a, c, g, or t
nnnnncsnnnnnnnnnnnnnnnnngcsccttcttgcmnngcaannrcggcagancctcggctcccttycytaaataagaggatttcttgtggctctcctccgcctatcctaaatggccggattagttattattctacccccattgctgttggtaccgtgataaggtacagttgttcaggtaccttccgcctcat

SEQ ID NO: 11 - DNA, 194 bp, Artificial Sequence
  Synthetic
  misc_feature (1)..(4): n is a, c, g, or t
  misc_feature (7)..(24): n is a, c, g, or t
  misc_feature (25)..(25): The residue at this position can be either G or C.
  misc_feature (29)..(29): n is a, c, g, or t
  misc_feature (33)..(33): The residue at this position can be either C or T.
  misc_feature (37)..(37): The residue at this position can be either A or C.
  misc_feature (38)..(39): n is a, c, g, or t
  misc_feature (44)..(45): n is a, c, g, or t
  misc_feature (54)..(54): n is a, c, g, or t
nnnngcnnnnnnnnnnnnnnnnnnscgcnctcytgcmnngcaanngcggcagancctcggctcccttccctaaataagaggatttcttgtggctctcctccgcctatcctaaatggccggattagttattattctacccccattgctgttggtaccgtgataaggtacagttgttcaggtaccttccgcctcat

SEQ ID NO: 12 - DNA, 13 bp, Artificial Sequence
  Synthetic
gcgagacggtggg

SEQ ID NO: 13 - DNA, 14 bp, Artificial Sequence
  Synthetic
gcgagacggtaggg

SEQ ID NO: 14 - DNA, 69 bp, Artificial Sequence
  Synthetic
ctgtacacccgaaccgcgctcttgcccagcaaaagcggcagaacctcggctcccttccctaaataagag

SEQ ID NO: 15 - protein, 9 aa, Artificial Sequence
  Synthetic
GGGGGGGGR

SEQ ID NO: 16 - protein, 16 aa, Artificial Sequence
  Synthetic
KMMMCGGGGGGGHHSW

SEQ ID NO: 17 - DNA, 22 bp, Artificial Sequence
  Synthetic
  misc_feature (1)..(1): The residue at this position is either C or U.
  misc_feature (13)..(13): The residue at this position is either U or A.
  misc_feature (20)..(20): The residue at this position is either U or C.
nccaacgggcccncuuuucncc

SEQ ID NO: 18 - DNA, 20 bp, Artificial Sequence
  Synthetic
  misc_feature (1)..(1): The residue at this position is either C or U.
  misc_feature (13)..(13): The residue at this position is either U or A.
  misc_feature (18)..(18): The residue at this position is either U or C.
nccaacgggcccncuucncc

SEQ ID NO: 19 - DNA, 20 bp, Artificial Sequence
  Synthetic
  misc_feature (1)..(1): The residue at this position is either C or U.
  misc_feature (13)..(13): The residue at this position is either U or A.
  misc_feature (18)..(18): The residue at this position is either U or C.
nccaacgggcccnccucncc

SEQ ID NO: 20 - DNA, 18 bp, Artificial Sequence
  Synthetic
  misc_feature (1)..(1): The residue at this position is either C or U.
  misc_feature (13)..(13): The residue at this position is either U or A.
  misc_feature (16)..(16): The residue at this position is either U or C.
nccaacgggcccnucncc

SEQ ID NO: 21 - DNA, 19 bp, Artificial Sequence
  Synthetic
  misc_feature (1)..(1): The residue at this position is either C or U.
  misc_feature (10)..(10): The residue at this position is either U or A.
  misc_feature (17)..(17): The residue at this position is either U or C.
nccauccccncuuuucncc

SEQ ID NO: 22 - DNA, 17 bp, Artificial Sequence
  Synthetic
  misc_feature (1)..(1): The residue in this position is either C or U.
  misc_feature (10)..(10): The residue in this position is either U or A.
  misc_feature (15)..(15): The residue in this position is either U or C.
nccauccccncuucncc

SEQ ID NO: 23 - DNA, 17 bp, Artificial Sequence
  Synthetic
  misc_feature (1)..(1): The residue at this position is either C or U.
  misc_feature (10)..(10): The residue at this position is either U or A.
  misc_feature (15)..(15): The residue at this position is either U or C.
nccauccccnccucncc

SEQ ID NO: 24 - DNA, 15 bp, Artificial Sequence
  Synthetic
  misc_feature (1)..(1): The residue at this position is either C or U.
  misc_feature (10)..(10): The residue at this position is either U or A.
  misc_feature (13)..(13): The residue at this position is either U or C.
nccauccccnucncc

SEQ ID NO: 25 - DNA, 18 bp, Artificial Sequence
  Synthetic
  misc_feature (1)..(1): The residue at this position is either C or U.
  misc_feature (9)..(9): The residue at this position is either U or A.
  misc_feature (16)..(16): The residue at this position is either U or C.
nccagcccncuuuucncc

SEQ ID NO: 26 - DNA, 16 bp, Artificial Sequence
  Synthetic
  misc_feature (1)..(1): The residue at this position is either C or U.
  misc_feature (9)..(9): The residue at this position is either U or A.
  misc_feature (14)..(14): The residue at this position is either U or C.
nccagcccncuucncc

SEQ ID NO: 27 - DNA, 16 bp, Artificial Sequence
  Synthetic
  misc_feature (1)..(1): The residue at this position is either C or U.
  misc_feature (9)..(9): The residue at this position is either U or A.
  misc_feature (14)..(14): The residue at this position is either U or C.
nccagcccnccucncc

SEQ ID NO: 28 - DNA, 14 bp, Artificial Sequence
  Synthetic
  misc_feature (1)..(1): The residue at this position is either C or U.
  misc_feature (9)..(9): The residue at this position is either U or A.
  misc_feature (12)..(12): The residue at this position is either U or C.
nccagcccnucncc

SEQ ID NO: 29 - DNA, 17 bp, Artificial Sequence
  Synthetic
  misc_feature (1)..(1): The residue at this position is either C or U.
  misc_feature (8)..(8): The residue at this position is either U or A.
  misc_feature (15)..(15): The residue at this position is either U or C.
nccacccncuuuucncc

SEQ ID NO: 30 - DNA, 15 bp, Artificial Sequence
  Synthetic
  misc_feature (1)..(1): The residue at this position is either C or U.
  misc_feature (8)..(8): The residue at this position is either U or A.
  misc_feature (13)..(13): The residue at this position is either U or C.
nccacccncuucncc

SEQ ID NO: 31 - DNA, 15 bp, Artificial Sequence
  Synthetic
  misc_feature (1)..(1): The residue at this position is either C or U.
  misc_feature (8)..(8): The residue at this position is either U or A.
  misc_feature (13)..(13): The residue at this position is either U or C.
nccacccnccucncc

SEQ ID NO: 32 - DNA, 13 bp, Artificial Sequence
  Synthetic
  misc_feature (1)..(1): The residue at this position is either C or U.
  misc_feature (8)..(8): The residue at this position is either U or A.
  misc_feature (11)..(11): The residue at this position is either U or C.
nccacccnucncc